(19) United States
(12) Patent Application Publication
(10) Pub. No.: US 2024/0342280 A1
(43) Pub. Date: Oct. 17, 2024

(54) OPTIMIZING T CELL DIFFERENTIATION STATE WITH MICRORNAS

(71) Applicants: THE TRUSTEES OF THE UNIVERSITY OF PENNSYLVANIA, Philadelphia, PA (US); University of Miami, Miami, FL (US)

(72) Inventors: Erietta Stelekati, Miami, FL (US); E. John Wherry, Havertown, PA (US); Joseph A. Fraietta, Vorhees, NJ (US)

(21) Appl. No.: 18/293,729

(22) PCT Filed: Aug. 9, 2022
(86) PCT No.: PCT/US22/74695

§ 371 Date: Jan. 30, 2024

Related U.S. Application Data
(60) Provisional application No. 63/231,140, filed on Aug. 9, 2021.

(51) Int. Cl. A61K 39/00; A61P 35/00; C07K 14/705; C07K 14/725; C07K 16/28; C12N 5/0783; C12N 15/113

(52) U.S. Cl. CPC A61K 39/4611; A61K 39/4631; A61K 39/464412; A61P 35/00; C07K 14/7051; C07K 14/70517; C07K 14/70578; C07K 16/2803; C12N 5/0636; C12N 15/113; A61K 2239/22; C07K 2317/53; C07K 2319/02; C07K 2319/03; C12N 2310/141; C12N 2510/00

(57) ABSTRACT

The current invention includes compositions and methods comprising immune effector cells modified to express miR-29a for the purpose of resisting immune exhaustion. In various embodiments the transmembrane domain comprises a transmembrane region of a protein selected from the group consisting of a type I transmembrane protein, an alpha chain of a T cell receptor, a beta chain of a T cell receptor, a zeta chain of a T cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9.

### CROSS-REFERENCE TO RELATED APPLICATION

The present application is entitled to priority under 35 U.S.C. § 119 (e) to U.S. Provisional Patent Application No. 63/231,140 filed Aug. 9, 2021, which is hereby incorporated by reference in its entirety herein.

### STATEMENT REGARDING FEDERALLY SPONSORED RESEARCH OR DEVELOPMENT

This invention was made with government support under AI105343, AI117950 and AI149680 awarded by National Institutes of Health. The government has certain rights in the invention.

### BACKGROUND OF THE INVENTION

CD8 T cell exhaustion, a cell differentiation state of sub-optimal function, is the underlying detrimental factor for chronic infections and cancer. Persistent antigen exposure during chronic infections and cancer prohibits the formation of functional memory CD8 T cells (T_{MEM}) that provide long-term protection and, instead, drives the differentiation of exhausted CD8 T cells (T_{EX}). T_{EX} have a very distinct transcriptional and epigenetic profile that distinguishes them from other functional CD8 T cell subsets. T_{EX} cannot persist long-term, have impaired functionality and limited cytokine production, and fail to provide long-term protection. Therefore, improving T_{EX} persistence, function, and protective capacity is the key for reinvigorating the immune response against chronic pathogens and tumors.

The potential to reinvigorate T_{EX} and direct the host T cells against tumor cells has revolutionized cancer treatments with immunotherapy. Clinically approved strategies to enhance host anti-tumor CD8 T cell responses include blocking of checkpoint inhibitors, such as PD-1, and adoptive transfer of engineered Chimeric Antigen Receptor (CAR) T cells directly targeting cancer cells. Despite some clinical success, a major limitation for both of these immunotherapy strategies remains the inability to redirect CD8 T cell differentiation, alter the epigenetic signature of exhaustion, generate T_{MEM} and induce long-term persistence of CD8 T cells, the hallmark of a successful, long-lasting adaptive immune response.

MicroRNAs (miRs) are short non-coding RNAs that can have a profound impact on a molecular pathway by targeting several molecules in the same pathway. miRs have been implicated in redirecting immune cell fate decisions including regulating genetic pathways that induce or oppose the differentiation of T cell exhaustion.

A need exists for novel compositions and methods that utilize microRNA-regulated pathways to optimize T cell development and function in order to enhance immunotherapies to treat diseases including cancer and chronic infections. The present invention addresses this need.

### SUMMARY OF THE INVENTION

In one aspect, the invention includes a modified immune cell or precursor cell thereof, comprising a nucleic acid encoding a chimeric antigen receptor (CAR) and a miR-29a microRNA, wherein the CAR comprises: an antigen binding domain domain that specifically binds a tumor associated antigen (TAA), a transmembrane domain, and an intracellular domain.

In certain embodiments, the transmembrane domain comprises a transmembrane region of a protein selected from the group consisting of a type I transmembrane protein, an alpha chain of a T cell receptor, a beta chain of a T cell receptor, a zeta chain of a T cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In certain embodiments, the transmembrane domain comprises a CD8 transmembrane region.

In certain embodiments, the intracellular domain comprises a costimulatory domain of a protein selected from the group consisting of a TNFR superfamily member, CD27, CD28, 4-1BB (CD137), OX40 (CD134), CD30, CD40, PD-1, ICOS, lymphocyte function-associated antigen-1 (LFA-1), CD2, CD5, CD7, LIGHT, NKG2C, B7-H3, a ligand that specifically binds with CD83, DAP10, DAP12, Lck, Fas, and any derivative or variant thereof. In certain embodiments, wherein the costimulatory domain is 4-1BB (CD137).

In certain embodiments, the intracellular domain comprises a signaling domain of a protein selected from the group consisting of CD3 zeta, FcyRIII, FcsRI, a cytoplasmic tail of an Fc receptor, an immunoreceptor tyrosine-based activation motif (ITAM) bearing cytoplasmic receptor, TCR zeta, FcR gamma, FcR beta, CD3 gamma, CD3 delta, CD3 epsilon, CD5, CD22, CD79a, CD79b, and CD66d. In certain embodiments, the intracellular domain comprises a signaling domain of CD3 zeta.

In certain embodiments, the CAR further comprises a hinge domain. In certain embodiments, the hinge domain is from a protein selected from the group consisting of an Fc fragment of an antibody, a hinge region of an antibody, a CH2 region of an antibody, a CH3 region of an antibody, an artificial spacer sequence, an amino acid hinge sequence of CD8, and any combination thereof. In certain embodiments, the hinge domain is a CD8 hinge domain.

In certain embodiments, the modified cell is a modified T cell. In certain embodiments, the modified immune cell is autologous.

In certain embodiments, the tumor-associated antigen is CD19.

In certain embodiments, the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Another aspect of the invention includes a modified T cell comprising a nucleic acid encoding a chimeric antigen receptor (CAR) and a miR-29a microRNA, wherein the CAR comprises: an antigen binding domain domain that specifically binds a tumor associated antigen (TAA), a transmembrane domain, and an intracellular domain.

In certain embodiments, the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Another aspect of the invention includes an isolated nucleic acid sequence encoding a chimeric antigen receptor (CAR) and a miR-29a microRNA, wherein the CAR comprises: an antigen binding domain domain that specifically binds a tumor associated antigen (TAA), a transmembrane domain, and an intracellular domain.

In certain embodiments, the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Another aspect of the invention includes an expression construct comprising any of the isolated nucleic acids contemplated herein. In certain embodiments, the expression construct further comprises an EF-1α promoter. In certain embodiments, the expression construct further comprises a woodchuck hepatitis virus posttranscriptional regulatory element (WPRE). In certain embodiments, the expression construct is a viral vector selected from the group consisting of a retroviral vector, a lentiviral vector, an adenoviral vector, and an adeno-associated viral vector. In certain embodiments, the expression construct is a lentiviral vector. In certain embodiments, the expression construct is a self-inactivating lentiviral vector.

Another aspect of the invention includes a method of treating cancer in a subject in need thereof, comprising administering to the subject a therapeutically effective composition comprising any of the modified immune cells contemplated herein.

Another aspect of the invention includes a method of treating a cancer in a subject in need thereof. The method comprises administering to the subject a therapeutically effective amount of a composition comprising a modified T cell comprising a nucleic acid encoding a chimeric antigen receptor (CAR) and a miR-29a microRNA, wherein the CAR comprises an antigen binding domain domain that specifically binds a tumor associated antigen (TAA), a transmembrane domain, and an intracellular domain.

In certain embodiments, the modified T cell is autologous.

In certain embodiments, the tumor associated antigen is CD19.

Another aspect of the invention includes an isolated T cell compising a nucleic acid vector encoding a miR29-29a which is operably linked to a promoter.

In certain embodiments, the promoter is constitutive. In certain embodiments, the promoter is inducible. In certain embodiments, the promoter drives the expression of miR-29a such that the function of the isolated T cell is altered.

In certain embodiments, the T cell is a CD8 T cell.

In certain embodiments, the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Another aspect of the invention includes a method of enhancing an immune response to an immunotherapy in a subject in need thereof, the method comprising contacting one or more immune effector cells of the subject with a nucleic acid vector encoding a miR-29a microRNA prior to treatment with the immunotherapy such that the immune effector cells express a high level of miR-29a microRNA as compared to uncontacted immune effector cells, thereby enhancing the effect of the immunotherapy.

In certain embodiments, the immune effector cells are T cells. In certain embodiments, the T cells are CD8+ T cells.

In certain embodiments, the high level of miR-29a renders the immune effectors cells resistant to immune exhaustion.

Another aspect of the invention includes a method of treating a chronic infection in a subject in need thereof, the method comprising contacting one or more immune effector cells of the subject with a nucleic acid vector encoding a miR-29a microRNA such that the immune effector cells express a high level of miR-29a microRNA as compared to uncontacted immune effector cells, thereby treating the chronic infection.

In certain embodiments, the immune effector cells are T cells. In certain embodiments, the T cells are CD8+ T cells.

In certain embodiments, the nucleic acid vector comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1C illustrate that miR-29a is a key memory CD8 T cell-specific microRNA dysregulated during exhaustion. C57/BL6 mice were infected with LCMV Armstrong (“acute”) or LCMV clone 13 (“chronic”). At d8 and d30 p.i. LCMV Db gp-33 specific CD8 T cells were purified from spleens and their miR profile was examined. As a control, CD8 T cells from naïve mice were purified (“T_{N}”). FIG. 1A: PCA between T_{N}, T_{EFF} and T_{MEM}. Heatmap and Venn diagram showing the differentially expressed (DE) miRs with FDR<0.05. FIG. 1B: PCA between T_{N}, T_{EE} and T_{MEM}. Heatmap and Venn diagram showing the differentially expressed (DE) miRs with FDR<0.05. FIG. 1C: The DE miRs between CD8 T cells responding to acute and chronic infection were used to create a list of predicted mRNA targets using Ingenuity Pathway Analysis. A list of DE mRNAs between CD8 T cells responding to acute and chronic infection was created. The DE mRNA list was used to filter the miRNA target list and select only the miRNA targets that were DE during the same timepoint but in the opposite direction of the miRNA. The filtered miRNA target list together with the list of DE miRs were then used to create a network of miRs and their predicted targets that were DE between acute and chronic infection at d30 p.i.

FIGS. 2A-2F illustrate that miR-29a attenuates CD8 T cell exhaustion. CD45.1+P14 CD8 T cells were transduced with either control-VEX RV (ctrl) or miR-29a OE-VEX RV (miR) and adoptively transferred to CD45.2+ recipient mice that were infected with LCMV clone 13 at 24 hrs earlier. FIG. 2A: Experimental design. FIGS. 2B-2C: Frequency and number of donor VEX+P14 cells in spleens. FIG. 2D: Expression of inhibitory receptors on VEX+P14 cells. FIG. 2E: MIP-1α and GzmB production by VEX+P14 cells. FIG. 2F: Cytokine production by VEX+P14 cells. Each data point represents an independent mouse. Representative results of at least 3 independent experiments with at least 11 mice per group.

FIGS. 3A-3I illustrate that miR-29a instructs a memory-like CD8 T cell transcriptional profile during chronic infection. P14 cells were transduced with miR-29a-OE (miR) or control (ctrl) RV and adoptively transferred as described in FIG. 2A. At d30 p.i., VEX+P14 cells were sorted and RNASeq was performed. FIG. 3A: Heatmap shows DE transcripts with FDR<0.05. FIGS. 3B-3C: GSEA was performed for gene signatures obtained from MSigDB (dataset: GSEA 9650). FIG. 3D: Percentage of pathways from each MSigDB database enriched (with FDR<0.05) in control or miR-29a-OE cells. Databases: 1: Hallmark, 2: KEGG, 3: Biocarta, 4: GO Molecular Process, 5: GO Cellular Component, 6: GO Molecular Function, 7: Transcription Factors GTRD, 8: MicroRNA predicted targets. FIG. 3E: Hallmark pathways enriched in control versus miR-29a OE P14 CD8 T cells. FIGS. 3F-3H: GSEA plots for datasets: FIG. 3F: “Inflammatory Response (Hallmark)”, FIG. 3G: “Antigen Response (Goldrath)”, FIG. 3H: AP-1 (PID) and NFAT (PID). FIG. 3I: Network analysis for genes DE between miR-29a OE and control with FDR<0.05.

FIGS. 4A-4D illustrate that miR-29a promotes memory-like CD8 T cell responses in chronic infection. CD45.1+P14 CD8 T cells were transduced with either control-VEX RV (ctrl) or miR-29a OE-VEX RV (miR) and adoptively transferred as shown in FIG. 2A. FIG. 4A: Percentages of terminal effector and memory precursor P14 cells (gated on VEX+P14 cells). FIG. 4B: Intracellular expression of TCF-1 and surface Ly 108. FIG. 4C-4D: At d34 p.i. transduced VEX+ and non-transduced VEX-P14 cells were sorted from the spleens of donor mice. 50,000 sorted VEX+ or VEX− P14 cells were separately adoptively transferred to congenic recipient mice that were infected with LCMV V35A 35 days prior. Recipient mice were then challenged with flu PR8-gp33. Secondary expansion of transferred P14 cells was analyzed on 9d post flu infection. FIG. 4D: Absolute numbers of transferred P14 cells were evaluated in lungs, mediastinal lymph nodes (mLN) and spleens on d9 post flu infection.

FIGS. 5A-5B illustrate that miRs are differentially expressed during CD8 T cell differentiation in acute and chronic infection. C57/BL6 mice were infected with either LCMV Armstrong (“acute”) or LCMV clone 13 (“chronic”). At d8 and at d30 p.i. LCMV Db gp-33 specific CD8 T cells were purified from spleens and their miR profile was examined using Affymetrix microRNA 2.0 array. As a control, CD8 T cells from naïve mice were purified (“T_{N}”). FIG. 5A: Venn diagrams showing the differentially expressed miRs (DEM: FDR<0.05) between T_{N}, T_{EFF} and T_{MEM} and between T_{N}, T_{EE} and T_{EX}. FIG. 5B: Venn diagram showing DEM (FDR<0.05) between T_{EFF} VS T_{EE} and T_{MEM} VS T_{EX}. Shown are miRs upregulated in the first group of the pairwise comparison.

FIGS. 6A-6B illustrate that key transcriptional pathways are implicated in miR differential regulation between acute and chronic infection. C57/BL6 mice were infected with LCMV Armstrong or clone 13. At d8 and at d30 p.i. LCMV Db gp-33 specific CD8 T cells were purified from spleens and their miR profile was examined as in FIGS. 1A-1C. FIG. 6A: A network of miRs and their predicted targets that were DE between acute and chronic infection at d8 p.i. was constructed using the filtered miR-mRNA target list from FIG. 2B. FIG. 6B: Upstream regulators of DEM and target DEG from the filtered miR-mRNA target list from FIG. 2B at d8 and at d30 p.i.

FIGS. 7A-7C illustrate that miR-29a induces a transcriptional profile similar to T_{MEM} and antagonizes a miR-155-controlled transcriptional program. RNASeq was performed using control or miR-29a-OE VEX+P14 from d30 p.i. with LCMV clone 13. FIG. 7A: GSEA for a geneset of predicted miR-29a targets (from MSigDB). FIG. 7B: GSEA for a geneset of T_{EFF} VS T_{MEM} (from MSigDB). FIG. 7C: GSEA for a geneset of genes upregulated upon miR-155-OE (from Stelekati E, et al. Proc Natl Acad Sci USA. 2022: 119 (17): e2106083119).

FIGS. 8A-8C illustrate that miR-29 overexpression enhances CD8 T cell responses during acute infection. CD45.1+P14 CD8 T cells were transduced with either control-VEX RV (ctrl) or miR-29a OE-VEX RV (miR) and adoptively transferred to CD45.2+ recipient mice that were infected with LCMV Armstrong at 24 hrs earlier.

FIG. 8A: Expansion of transferred P14 cells at d35 p.i. in spleens. FIG. 8B: Surface expression of CD127 and KLRG1 on VEX+P14 cells. FIG. 8C: Cytokine production by VEX+P14 cells after ex vivo 5 hr re-stimulation with gp-33 peptide.

FIGS. 9A-9D are a sequence map illustrating a CD19BBZ-miR29a chimeric antigen receptor (CAR) construct.

FIG. 10 illustrates the ability of CAR19-miR29 expressing T cells to lyse target cells in an in vitro cytotoxicity assay.

FIG. 11 is a table illustrating differentially expressed transcripts with FDR<0.05 between ctrl and miR-29a-OE CD8 T cells.

FIG. 12 is a table illustrating pathway analysis reveals a role for miR-29a in regulating ribosomal pathways.

FIG. 13 is a table illustrating that miR-29a abrogates the response to antigen signaling and inflammation.

FIG. 14 is a table illustrating that GSEA analysis implicates regulation of major transcription factors by miR-29a.

FIGS. 15A-15D illustrate the finding that miR-29a synergizes with anti-PD-L1 to promote memory differentiation.

FIGS. 16A-16C illustrate the finding that miR-29a synergizes with anti-PDL1 to promote the differentiation of the progenitor subset that responds to immunotherapy.

FIGS. 17A-17D illustrate the finding that miR-29a synergizes with anti-PDL1 to promote long-term persisting CD8 T cell responses.

FIG. 18 illustrates the finding that miR-29a synergizes with anti-PD-L1 therapy to sustain memory-like T cells and enhances effector functions.

FIG. 19 illustrates the finding that miR-29a and anti-PD-L1 therapy retains exhaustion-associated receptors at low levels after the cessation of PD-L1 blockade.

FIG. 20 illustrates the finding that CD8+ T cells that overexpress miR-29 can limit tumor growth (B16 melanoma).

FIGS. 21A-21B illustrate the finding that miR-29a promotes anti-tumor responses in an orthotopic pancreatic tumor model.

FIG. 22 illustrates the finding that miR-29a promotes anti-tumor responses in a hepatocellular carcinoma tumor model.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

“Allogeneic” refers to any material derived from a different animal of the same species.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY: Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

The term “chimeric antigen receptor” or “CAR.” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CAR has specificity to a selected target, for example CD19. CARs may also comprise an intracellular activation domain, a transmembrane domain and an extracellular domain comprising an antigen binding region.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes: i.e., they are multivalent. In general, an epitope is roughly about 10 amino acids and/or sugars in size. In certain exemplary embodiments, the epitope is about 4-18 amino acids, about 5-16 amino acids, about 6-14 amino acids, about 7-12 amino acids, or about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression: other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit: e.g., if a position in each of two DNA molecules is occupied by adenine, then they are homologous at that position. The homology between two sequences is a direct function of the number of matching or homologous positions: e.g., if half (e.g., five positions in a polymer ten subunits in length) of the positions in two sequences are homologous, the two sequences are 50% homologous: if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525, 1986; Reichmann et al., Nature, 332:323-329, 1988: Presta, Curr. Op. Struct. Biol., 2:593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions: e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions: e.g., if half (e.g., five positions in a polymer ten amino acids in length) of the positions in two sequences are identical, the two sequences are 50% identical: if 90% of the positions (e.g., 9 of 10), are matched or identical, the two amino acids sequences are 90% identical.

The term “immunoglobulin” or “Ig.” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

The term “immunostimulatory” is used herein to refer to increasing overall immune response.

The term “immunosuppressive” is used herein to refer to reducing overall immune response.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells: they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, e.g., a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species: for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. In exemplary embodiments, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai virus vectors, adenovirus vectors, adeno-associated virus vectors, retrovirus vectors, lentivirus vectors, and the like.

“Xenogeneic” refers to any material derived from an animal of a different species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based on the discovery that the microRNA miR-29a regulates the development of immune exhaustion in T cells and that T cells modified to express elevated levels of miR-29a are resistant to developing immune exhaustion and thus have better immune function than unmodified T cells. As such, the current invention provides isolated immune effector cells comprising nucleic acid vectors encoding miR-29a. The resistance to immune exhaustion can enhance T cell function in a variety of immunotherapeutic settings. Thus the current invention also provides for methods of enhancing immunotherapies, methods of treating cancer, and methods of treating chronic infections all comprising modifying immune effector cells, including T cells, to express miR-29a. The invention also provides chimeric antigen receptors (CARs) specific for tumor-associated antigens comprising a nucleic acid encoding miR-29a, thereby enhancing the function and efficiacy of immune effector cells comprising the CAR.

### Immune Exhaustion of T Cells

Immune exhaustion refers to the loss of effector function and proliferative capacity in T cells, especially memory T cells. Following antigen recognition by the T cell receptor and activation through “second signal” co-stimulatory molecules, naïve T cells undergo a period of rapid proliferation and acquisition of effector functions associated with changes in surface phenotype and gene expression. Most of the activated T cells differentiate into short-lived effector T cells, while a smaller number become long-lived central memory T cells. For CD8+ T cells, naïve T cells (T_{N}) undergo clonal expansion and differentiate into effector CD8 T cells (T_{EFF}). Upon antigen elimination, antigen-experienced CD8 T cells differentiate into long-lasting memory CD8 T cells (T_{MEM}) that provide protection upon subsequent reinfection.

In contrast, during chronic infections, or in situations of persistent antigen stimulation such as certain immunotherapies, the generation of optimal T_{MEM} fails to occur and instead development results in T cell exhaustion. Exhausted CD8 T cells (T_{EX}) produce limited cytokines and fail to protect upon secondary antigen challenge.

Transcriptional profiling has identified characteristic expression profiles for exhausted CD8 T cells, including high expression of inhibitory receptors, changes in signaling pathways, altered expression and use of transcription factors, and bioenergetic alterations including reduced expression of ribosomal subunit genes. Moreover, exhausted CD8 T cells have a distinct open chromatin landscape compared to effector and memory T cells identifying exhausted T cells as a distinct branch of mature CD8 T cell differentiation.

The distinct transcriptional and epigenetic features of CD8 T cell exhaustion are found not only in chronic infections but also in other situations of constant or chronic antigen stimulation including tumor infiltrating CD8 T cells in mice and humans, suggesting that common pathways underline T cell exhaustion differentiation in different disease settings. As such, exhaustion has been found to limit the effectiveness of immunotherapies due to the functional impairment of otherwise therapeutic T cells. A nonlimiting example of T cell exhaustion limiting clinical efficiacy is the difficulty of treating solid cancers with CAR-expressing T cells.

Thus, in certain aspects, the current invention includes methods of modifying T cells to be resistant to the induction of exhaustion in order to prolong their efficiacy as immune effectors. In certain embodiments, the immune effectors of the invention are CD8+ T cells. In certain aspects, the current invention also includes methods of enhancing immunotherapeutic treatments comprising modifying immune effector cells, including CD8 T cells, such that they are resistant to exhaustion. In other aspects, such modified cells are used in methods of treating diseases such as cancer and chronic infections in subjects in need thereof.

MicroRNAs and miR-29a

MicroRNAs or miRNAs are a class of small, single-stranded non-coding RNA molecules found in most eukaryotic cells and some eukaryotic-tropic viruses. While not direcly coding for protein, miRNAs function to regulate gene expression through interaction with compelentary messenger RNAs (mRNAs). MiRs can exert powerful regulatory effects on specific biological pathways by simultaneously targeting several mRNAs in the same pathway. Mechanistically, miRNAs are typically transcribed as 100-1000 nucleotide (nt) primary miRNAs (pri-RNAs) by RNA polymerase II. miRNAs may be modified by 5′ capping and 3′ poly(A) tailing. The miRNA-encoding portion of the pri-miRNA forms a hairpin, which is cleaved by the dsRNA-specific ribonuclease Drosha and its cofactor DiGeorge syndrome critical region 8 (DGCR8), to form a pre-miRNA that is about 60-70 nt long. The pre-miRNA is further processed by Dicer and the trans-activator RNA-binding protein TRBP to yield a miRNA duplex containing two mature miRNAs (5′- and 3′-strand miRNAs). Each mature miRNA is about 22-23 nt in length.

Persistent TCR stimulation is a key factor leading to CD8 T cell exhaustion. Thus, two miRs induced upon TCR signaling, miR-31 and miR-155, were shown to regulate exhaustion. MiR-31 promotes exhaustion by increasing CD8 T cell sensitivity to type I IFN, whereas miR-155 promotes exhaustion during chronic viral infection, but also enhances long-term exhausted T cell persistence by targeting the AP-1 transcription factor Fosl2. This latter effect of miR-155 may represent an adaptation that allows T_{EX} to withstand the stress of constant TCR stimulation and persist long-term, contributing to partial disease containment.

MiR-29a is a member of a family of closely-related miRs (miR-29) that were originally identified as being associated with aggressiveness and poor prognosis of malignant cancers. Immunologically, the studies of the present disclosure identify miR-29a as a key T cell memory-associated miR. Enforced expression of miR-29a enhances virus-specific CD8 T cell responses to acutely resolved and chronic viral infections, antagonizes development of exhaustion and promotes T_{MEM}-like patterns of differentiation even during chronic infection. Mechanistically, miR-29a attenuates inflammatory and TCR signaling during conditions of exhaustion and targets key transcriptional pathways associated with exhaustion, including Tox and AP-1.

In certain aspects, the current invention includes the modification of immune effector cells, including CD8+ T cells, to express relatively higher levels of miR-29a, thereby enabling the cells to resist differentiation into T_{EX} cells. In some embodiments, these modified cells are used in methods of treating diseases including cancer and chronic infection. In some embodiments, such modifications of immune effector cells, including CD8+ T cells, are used in methods of enhancing immunotherapy. In some preferred embodiments, the invention includes the combination of miR-29a expression with chimeric antigen receptor (CAR) expression in modified T cells, thereby improving the in vivo function and persistence of the CAR-expressing cells. In certain embodiments, the invention includes modified immune cells (e.g., T cells) comprising a nucleic acid encoding a CAR and a miR-29a. In certain embodiments, the miR-29a is encoded by a nucleotide sequence 80%, 85%, 90%, 95%, 96%, 97%, 98%, 99%, or 100% identical to any of SEQ ID NOs: 28, 29, 30, 31, 32, and 33.

In certain embodiments, the invention includes modified immune cells (e.g., T cells) comprising a nucleic acid encoding a CAR and a miR-29b1. In certain embodiments, the miR-29b1 is encoded by a nucleotide sequence 80%, 85%, 90%, 95%, 96%, 97%, 98%, 99%, or 100% identical to any of SEQ ID NOs: 34, 35, 36, 37, 38, and 39.

In certain embodiments, the invention includes modified immune cells (e.g., T cells) comprising a nucleic acid encoding a CAR, a miR-29a, and a miR-29b1. In certain embodiments, the miR-29a and miR-29b1 are encoded by a nucleotide sequence 80%, 85%, 90%, 95%, 96%, 97%, 98%, 99%, or 100% identical to SEQ ID NO: 27.

miR-29a and b1
(SEQ ID NO: 27)
ctcgaggcatgctctcccatcaataacaaattcagtgacatcagtttatgaatatatgaaatttgccaaagctctgtttagaccact
gagtaactcacagctaggtttcaacttttcctttctaggttgtcttgggtttattgtaagagagcattatgaagaaaaaaatagatca
taaagcttcttcaggaagctggtttcatatggtggtttagatttaaatagtgattgtctagcaccatttgaaatcagtgttcttggggg
agaccagctgcgctgcactaccaacagcaaaagaagtgaatgggacagctctgaagtatttgaaagcaacagcaggatggc
tgtgagaacctgcctcacatgtagctgaccccttcctcacccctgccaacagtggtggcatatatcacaaatggcagtcaggtc
tctgcactggcggatccaactgtgatcgaaagttttccaaaaataagttgtgtctgtattgaacatgaacagactttcttcttgtcatt
attctctaacaatactgcataacaattatttgcatacatttgcattgcattaagtattctaagtaatctagagacgatttaaagtatacg
ggaggatgtgtgtaggttgtatgcaaatactacaccattttctatcagagacttgagcatctgtggattttggtatccaaggggctt
tctggaaccaatccctcaaggataccaagggatgaatgtaattgtacaggatatcgcattgttggaattttatacttctttgtggaat
aaacctatagcacttaatagatagtacagactcattccattgtgcctgggttaaagagcccaatgtatgctggatttagtaagattt
gggccctcccaaccctcacgaccttctgtgaccccttagaggatgactgatttcttttggtgttcagagtcaatataattttctagc
accatctgaaatcggttataatgattggggaagagcaccatgatgctgactgctgagaggaaatgtattggtgaccgttggggc
catggacaagaactaagaaaacaaatgcaaagcaataatgcaaaggtgatttttcttcttccagtttctaagttgaatttcactgac
ctgaattgcatgtggtctcgag
human miR-29a stem-loop (hsa-miR-29a)
RNA sequence (SEQ ID NO: 28):
AUGACUGAUUUCUUUUGGUGUUCAGAGUCAAUAUAAUUUUCUAGCACCA
UCUGAAAUCGGUUAU
DNA sequence
(SEQ ID NO: 29)
ATGACTGATTTCTTTTGGTGTTCAGAGTCAATATAATTTTCTAGCACCATCTG
AAATCGGTTAT
human miR-29a mature sequence (hsa-miR-29-5p)
RNA sequence (SEQ ID NO: 30):
ACUGAUUUCUUUUGGUGUUCAG
DNA sequence (SEQ ID NO: 31):
ACTGATTTCTTTTGGTGTTCAG
human miR-29a mature sequence (hsa-miR-29-3p)
RNA sequence (SEQ ID NO: 32):
UAGCACCAUCUGAAAUCGGUUA
DNA sequence (SEQ ID NO: 33):
TAGCACCATCTGAAATCGGTTA
human miR-29b1 stem-loop (hsa-miR-29b1)
RNA sequence (SEQ ID NO: 34):
CUUCAGGAAGCUGGUUUCAUAUGGUGGUUUAGAUUUAAAUAGUGAUUGU
CUAGCACCAUUUGAAAUCAGUGUUCUUGGGGG
DNA Sequence (SEQ ID NO: 35):
CTTCAGGAAGCTGGTTTCATATGGTGGTTTAGATTTAAATAGTGATTGTCTA
GCACCATTTGAAATCAGTGTTCTTGGGGG
human miR-29b1 mature sequence (hsa-miR-29b1-5p)
RNA Sequence (SEQ ID NO: 36):
GCUGGUUUCAUAUGGUGGUUUAGA
DNA Sequence (SEQ ID NO: 37):
GCTGGTTTCATATGGTGGTTTAGA
human miR-29b1 mature sequence (hsa-miR-29b-3p)
RNA Sequence (SEQ ID NO: 38):
UAGCACCAUUUGAAAUCAGUGUU
DNA Sequence (SEQ ID NO: 39):
TAGCACCATTTGAAATCAGTGTT

### Modified Immune Cells

The present invention provides modified immune cells or precursor cells thereof (e.g., a modified T cell, a modified NK cell, a modified NKT cell), comprising a nucleic acid encoding a chimeric antigen receptor (CAR) and a microRNA. Accordingly, such modified cells possess the specificity directed by the CAR that is expressed therein. For example, a modified cell of the present invention comprising a tumor associated antigen specific CAR possesses specificity for tumor associated antigen on a target cell.

A modified cell comprising any CAR comprising any antigen binding domain, any hinge, any transmembrane domain, any intracellular domain, and any intracellular signaling domain, known in the art or described herein is envisioned, and can readily be understood and made by a person of skill in the art in view of the disclosure herein.

In certain embodiments, the invention includes a modified immune cell (e.g., T cell) comprising a nucleic acid encoding a CAR and a miR-29a microRNA. In certain embodiments, the miR-29a is encoded by a nucleotide sequence 80%, 85%, 90%, 95%, 96%, 97%, 98%, 99%, or 100% identical to any of SEQ ID NOs: 28, 29, 30, 31, 32, and 33.

In certain embodiments, the invention includes s modified immune cell (e.g., T cell) comprising a nucleic acid encoding a CAR and a miR-29b1 microRNA. In certain embodiments, the miR-29b1 is encoded by a nucleotide sequence 80%, 85%, 90%, 95%, 96%, 97%, 98%, 99%, or 100% identical to any of SEQ ID NOs: 34, 35, 36, 37, 38, and 39.

In certain embodiments, the invention includes a modified immune cell (e.g., T cell) comprising a nucleic acid encoding a CAR, a miR-29a, and a miR-29b1. In certain embodiments, the miR-29a and miR-29b1 are encoded by a nucleotide sequence 80%, 85%, 90%, 95%, 96%, 97%, 98%, 99%, or 100% identical to SEQ ID NO: 27.

In some embodiments, the modified cell is an immune cell or precursor cell thereof. In an exemplary embodiment, the modified cell is a T cell. In an exemplary embodiment, the modified cell is an autologous cell. In an exemplary embodiment, the modified cell is an autologous immune cell or precursor cell thereof. In an exemplary embodiment, the modified cell is an autologous T cell.

### Chimeric Antigen Receptor (CAR)

The present invention provides compositions and methods for modified immune cells or precursor cells thereof, e.g., modified T cells, comprising a chimeric antigen receptor (CAR) having affinity for a tumor associated antigen. A subject CAR of the invention comprises an antigen binding domain (e.g., a tumor associated antigen binding domain), a transmembrane domain, and an intracellular domain. In certain embodiments, the intracellular domain comprises a costimulatory domain and an intracellular signaling domain. A subject CAR of the invention may optionally comprise a hinge domain. Accordingly, a subject CAR of the invention comprises an antigen binding domain (e.g., a tumor associated antigen binding domain), a hinge domain, a transmembrane domain, and an intracellular domain. In some embodiments, each of the domains of a subject CAR is separated by a linker.

The antigen binding domain may be operably linked to another domain of the CAR, such as the transmembrane domain, the costimulatory signaling domain, or the intracellular signaling domain, each described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding a costimulatory domain, and further operably linked to a fourth nucleic acid sequence encoding an intracellular signaling domain.

In one aspect, the invention includes a chimeric antigen receptor (CAR) that specifically binds a tumor associated antigen, comprising: a tumor associated antigen-specific antigen binding domain, optionally a hinge domain, a transmembrane domain, a costimulatory domain, and an intracellular signaling domain.

In one exemplary embodiment, the invention includes a chimeric antigen receptor (CAR) that specifically binds CD19, comprising: a CD19-specific antigen binding: a CD8 hinge domain: a CD8 transmembrane domain: a 4-1BB costimulatory domain; and a CD3 zeta intracellular signaling domain.

In certain embodiments, the genetically modified cell is a T cell. In certain embodiments, the genetically modified cell is a CD8+ T cell.

Accordingly, in one exemplary embodiment, provided herein is a genetically modified T cell comprising nucleic acid encoding a) a chimeric antigen receptor (CAR) that specifically binds a tumor associated antigen, comprising: a tumor associated-specific antigen binding domain, an optional hinge domain, a transmembrane domain, a costimulatory signaling domain, an intracellular signaling domain, and encoding b) a miR-29a microRNA, and wherein the cell expresses the CAR and miR-29a.

Accordingly, in one exemplary embodiment, provided herein is a genetically modified T cell comprising nucleic acid encoding a) a chimeric antigen receptor (CAR) that specifically binds CD19, comprising: a CD19-specific antigen binding domain: a CD8 hinge domain: a CD8 transmembrane domain: a 4-1BB costimulatory domain: a CD3 zeta intracellular signaling domain; and encoding b) a miR-29a microRNA, wherein the cell expresses the CAR and miR-29a.

In certain embodiments of the invention, the CAR and miR-29a are encoded by a vector comprising the nucleotide sequence of SEQ ID NO: 1.

### Tumor Associated Antigens

In certain aspects, the current invention includes a CAR or CARs that comprise antigen-binding domains specific for tumor associated antigens. A tumor associated antigen is a protein, polysaccharide, or other antigen substance produced largely or exclusively by certain tumor cells that triggers an immune response in a subject. Tumor associated antigens are useful targets for directing various cytotoxic therapeutic strategies, including CAR-expressing T cells, to tumor cells where they can be killed or eliminated preferentially while leaving surrounding normal tissue largely unaffected.

In certain aspects, the current invention includes a cell comprising a CAR comprising antigen-binding domains specific for tumor associated antigens, and the cell further comprises a nucleic acid encoding miR-29a. In this way, the expression of the miR-29a microRNA serves to confer resistance to the development of immune exhaustion of the cell expressing the CAR regardless of the target of the antigen-binding domain.

The skilled artisan would be able to select a tumor associated antigen appropriate for the type of tumor cells to be targeted by the CAR. Tumor associated antigens known to the art include, but are not limited to CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, and any combination thereof. In some preferred embodiments, the tumor associated antigen targetd by the CAR is CD19.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

In certain embodiments, the CAR of the invention comprises an antigen binding domain that binds to CD19. In certain embodiments, the antigen binding domain of the invention comprises an antibody or fragment thereof, that binds to a CD19 molecule. In certain exemplary embodiments, the antigen binding domain is an scFv antibody that binds to CD19. The choice of antigen binding domain depends upon the type and number of antigens that are present on the surface of a target cell. For example, the antigen binding domain may be chosen to recognize an antigen that acts as a cell surface marker on a target cell associated with a particular status of the target cell.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In an exemplary embodiment, a CAR of the present disclosure having affinity for CD19 on a target cell may comprise a CD19 binding domain. In some embodiments, the CD19 binding domain is a murine CD19 binding domain, e.g., the CD19 binding domain is of murine origin. In some embodiments, the CD19 binding domain is a humanized CD19 binding domain. In some embodiments, the CD19 binding domain is a human CD19 binding domain, e.g., the CD19 binding domain is of human origin.

In some embodiments, a CAR of the present disclosure may have affinity for one or more target antigens on one or more target cells. In some embodiments, a CAR may have affinity for one or more target antigens on a single target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through a polypeptide linker, an Fc hinge region, or a membrane hinge region.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In another embodiment, the antigen binding domain of the CAR is selected from the group consisting of a CD19 antibody or a fragment thereof. In some embodiments, the antigen binding domain is selected from the group consisting of an antibody, an antigen binding fragment (Fab), and a single-chain variable fragment (scFv). In some embodiments, a CD19 binding domain of the present invention is selected from the group consisting of a CD19-specific antibody, a CD19-specific Fab, and a CD19-specific scFv. In one embodiment, a CD19 binding domain is a CD19-specific antibody. In one embodiment, a CD19 binding domain is a CD19-specific Fab. In one embodiment, a CD19 binding domain is a CD19-specific scFv.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., tumor associated antigen binding domain) comprises an scFv having the configuration from N-terminus to C-terminus, VH-linker-VL. In some embodiments, the antigen binding domain (e.g., tumor associated antigen binding domain) comprises an scFv having the configuration from N-terminus to C-terminus, VL-linker-VH. Those of skill in the art would be able to select the appropriate configuration for use in the present invention.

The linker is typically rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80 (6): 1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 2), (GGGS)ₙ (SEQ ID NO: 3), and (GGGGS)ₙ (SEQ ID NO: 4), where n represents an integer of at least 1. Exemplary linker sequences can comprise amino acid sequences including, without limitation, GGSG (SEQ ID NO: 5), GGSGG (SEQ ID NO: 6), GSGSG (SEQ ID NO: 7), GSGGG (SEQ ID NO: 8), GGGSG (SEQ ID NO: 9), GSSSG (SEQ ID NO: 10), GGGGS (SEQ ID NO: 11), GGGGSGGGGSGGGGS (SEQ ID NO: 12) and the like. Those of skill in the art would be able to select the appropriate linker sequence for use in the present invention. In one embodiment, an antigen binding domain (e.g., tumor associated antigen binding domain) of the present invention comprises a heavy chain variable region (VH) and a light chain variable region (VL), wherein the VH and VL is separated by the linker sequence having the amino acid sequence GGGGSGGGGSGGGGS (SEQ ID NO: 13), which may be encoded by a nucleic acid sequence comprising the nucleotide sequence ggtggcggtggctcgggcggtggtgggtcgggtggcggcggatct (SEQ ID NO: 14).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27 (6): 455-51: Peter et al., J Cachexia Sarcopenia Muscle 2012 Aug. 12: Shieh et al., J Imunol 2009 183 (4): 2277-85: Giomarelli et al., Thromb Haemost 2007 97 (6): 955-63: Fife eta., J Clin Invst 2006 116 (8): 2252-61: Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chem 2003 25278 (38): 36740-7; Xie et al., Nat Biotech 1997 15 (8): 768-71: Ledbetter et al., Crit Rev Immunol 1997 17 (5-6): 427-55; Ho et al., BioChim Biophys Acta 2003 1638 (3): 257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region: Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some instances, the antigen binding domain may be derived from the same species in which the CAR will ultimately be used. For example, for use in humans, the antigen binding domain of the CAR may comprise a human antibody as described elsewhere herein, or a fragment thereof.

In an exemplary embodiment, a tumor antigen specific CAR of the present invention comprises a tumor associated antigen binding domain, e.g., a CD19-specific scFv. In one embodiment, the CAR of then invention is encoded by a nucleic acid sequence comprising the nucleotide sequence set forth in SEQ ID NO: 1.

In some embodiments, the CD19 binding CAR is encoded by a nucleic acid sequence comprising the nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleotide sequence set forth in SEQ ID NO: 1.

The antigen binding domain may be operably linked to another domain of the CAR, such as the transmembrane domain or the costimulatory signaling domain, both described elsewhere herein. In one embodiment, a nucleic acid encoding the antigen binding domain is operably linked to a nucleic acid encoding a transmembrane domain and a nucleic acid encoding a costimulatory signaling domain.

The antigen binding domains described herein, such as the antibody or fragment thereof that binds to a tumor associated antigen, can be combined with any of the transmembrane domains described herein, any of the intracellular domains or cytoplasmic domains described herein, or any of the other domains described herein that may be included in the CAR.

### Transmembrane Domain

With respect to the transmembrane domain, the CAR of the present invention can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3 (2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G)ₙ, glycine-serine polymers (including, for example, (GS)ₙ, (GSGGS)ₙ (SEQ ID NO: 2) and (GGGS)ₙ (SEQ ID NO: 3), where n is an integer of at least one), glycine-alanine polymers, alanine-serine polymers, and other flexible linkers known in the art. Glycine and glycine-serine polymers can be used: both Gly and Ser are relatively unstructured, and therefore can serve as a neutral tether between components. Glycine polymers can be used: glycine accesses significantly more phi-psi space than even alanine, and is much less restricted than residues with longer side chains (see, e.g., Scheraga, Rev. Computational. Chem. (1992) 2:73-142). Exemplary hinge regions can comprise amino acid sequences including, but not limited to, GGSG (SEQ ID NO: 5), GGSGG (SEQ ID NO: 6), GSGSG (SEQ ID NO: 7), GSGGG (SEQ ID NO: 8), GGGSG (SEQ ID NO: 9), GSSSG (SEQ ID NO: 10), and the like.

In some embodiments, the hinge region is an immunoglobulin heavy chain hinge region. Immunoglobulin hinge region amino acid sequences are known in the art: see, e.g., Tan et al., Proc. Natl. Acad. Sci. USA (1990) 87 (1): 162-166; and Huck et al., Nucleic Acids Res. (1986) 14 (4): 1779-1789. As non-limiting examples, an immunoglobulin hinge region can include one of the following amino acid sequences: DKTHT (SEQ ID NO: 15): CPPC (SEQ ID NO: 16): CPEPKSCDTPPPCPR (SEQ ID NO: 17) (see, e.g., Glaser et al., J. Biol. Chem. (2005) 280:41494-41503): ELKTPLGDTTHT (SEQ ID NO: 18): KSCDKTHTCP (SEQ ID NO: 19); KCCVDCP (SEQ ID NO: 20): KYGPPCP (SEQ ID NO: 21): EPKSCDKTHTCPPCP (SEQ ID NO: 22) (human IgG1 hinge): ERKCCVECPPCP (SEQ ID NO: 23) (human IgG2 hinge): ELKTPLGDTTHTCPRCP (SEQ ID NO: 24) (human IgG3 hinge): SPNMVPHAHHAQ (SEQ ID NO: 25) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 26): see, e.g., Yan et al., J. Biol. Chem. (2012) 287:5891-5897. In one embodiment, the hinge region can comprise an amino acid sequence derived from human CD8, or a variant thereof.

The transmembrane domain may be combined with any hinge domain and/or may comprise one or more transmembrane domains described herein.

The transmembrane domains described herein, such as a transmembrane region of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

Accordingly, a subject CAR of the present disclosure may comprise any of the transmembrane domains, hinge domains, or spacer domains described herein.

### Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a costimulatory signaling domain and an intracellular signaling domain. In certain embodiments, the intracellular domain comprises 4-1BB and CD3 zeta. In certain embodiments, the costimulatory signaling domain comprises 4-1BB. In certain embodiments, the intracellular signaling domain comprises CD3 zeta.

In one embodiment, the intracellular domain of the CAR comprises a costimulatory signaling domain which includes any portion of one or more co-stimulatory molecules, such as at least one signaling domain from CD2, CD3, CD8, CD27, CD28, OX40, ICOS, 4-1BB, PD-1, any derivative or variant thereof, any synthetic sequence thereof that has the same functional capability, and any combination thereof.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGBl, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33 (6): 651-653). Additionally, intracellular signaling domains may include signaling domains used by NK and NKT cells (see, e.g., Hermanson and Kaufman, Front. Immunol. (2015) 6:195) such as signaling domains of NKp30 (B7-H6) (see, e.g., Zhang et al., J. Immunol. (2012) 189 (5): 2290-2299), and DAP 12 (see, e.g., Topfer et al., J. Immunol. (2015) 194 (7): 3201-3212), NKG2D, NKp44, NKp46, DAP10), and CD3z.

Intracellular signaling domains suitable for use in a subject CAR of the present invention include any desired signaling domain that provides a distinct and detectable signal (e.g., increased production of one or more cytokines by the cell: change in transcription of a target gene: change in activity of a protein: change in cell behavior, e.g., cell death: cellular proliferation: cellular differentiation; cell survival; modulation of cellular signaling responses: etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5 (see, e.g., Gillis et al., Front. (2014) Immunol. 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP: TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcR gamma; fceRI gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D: CD3-DELTA: T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e. T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR.

Further, variant intracellular signaling domains suitable for use in a subject CAR are known in the art. The YMFM motif is found in ICOS and is a SH2 binding motif that recruits both p85 and p50alpha subunits of PI3K, resulting in enhanced AKT signaling. See, e.g., Simpson et al. (2010) Curr. Opin. Immunol., 22:326-332. In one embodiment, a CD28 intracellular domain variant may be generated to comprise a YMFM motif.

In one embodiment, the intracellular domain of a subject CAR comprises a 4-1BB costimulatory domain.

In one embodiment, the intracellular domain of a subject CAR comprises a CD3 zeta intracellular signaling domain.

The invention should be construed to include any CAR known in the art and/or disclosed herein. Exemplary CARs include, but are not limited to, those disclosed herein, those disclosed in U.S. Pat. Nos. 10,357,514B2, 10,221,245B2, 10,603,378B2, 8,916,381B1, 9,394,368B2, US20140050708A1, U.S. Pat. Nos. 9,598,489B2, 9,365,641B2, US20210079059A1, U.S. Pat. Nos. 9,783,591B2, WO2016028896A1, U.S. Pat. No. 9,446,105B2, WO2016014576A1, US20210284752A1, WO2016014565A2, WO2016014535A1, and U.S. Pat. No. 9,272,002B2, and any other CAR generally disclosed in the art.

### CAR Sequences

A subject CAR of the present invention may be a CAR having affinity for CD19. In one embodiment, the CD19 CAR of the present invention comprises a 4-1BB costimulatory domain and a CD3 zeta intracellular signaling domain.

Tolerable variations of the CAR will be known to those of skill in the art, while maintaining specific activity. For example, in some embodiments the CAR is encoded by a nucleic acid sequence comprising a nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleotide sequence set forth in SEQ ID NO: 1.

CD19BBZ-miR29a CAR (SEQ ID NO: 1): (comprising miR-29a and b1 (SEQ ID NO: 3)):
gtgcacgagtgggttacatcgaactggatctcaacagcggtaagatccttgagagttttcgccccgaagaacgttttccaatgat
gagcacttttaaagttctgctatgtggcgcggtattatcccgtattgacgccgggcaagagcaactcggtcgccgcatacactat
tctcagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttgca
caacatgggggatcatgtaactcgccttgatcgttgggaaccggagctgaatgaagccataccaaacgacgagcgtgacac
cacgatgcctgtagcaatggcaacaacgttgcgcaaactattaactggcgaactacttactctagcttcccggcaacaattaata
gactggatggaggcggataaagttgcaggaccacttctgcgctcggcccttccggctggctggtttattgctgataaatctgga
gccggtgagcgtgggtctcgcggtatcattgcagcactggggccagatggtaagccctcccgtatcgtagttatctacacgac
ggggagtcaggcaactatggatgaacgaaatagacagatcgctgagataggtgcctcactgattaagcattggtaactgtcag
accaagtttactcatatatactttagattgatttaaaacttcatttttaatttaaaaggatctaggtgaagatcctttttgataatctcatg
accaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactcttt
ttccgaaggtaactggcttcagcagagcgcagataccaaatactgttcttctagtgtagccgtagttaggccaccacttcaagaa
ctctgtagcaccgcctacatacctcgctctgctaatcctgttaccagtggctgctgccagtggcgataagtcgtgtcttaccggg
ttggactcaagacgatagttaccggataaggcgcagcggtcgggctgaacggggggttcgtgcacacagcccagcttggag
cgaacgacctacaccgaactgagatacctacagcgtgagctatgagaaagcgccacgcttcccgaagggagaaaggcgga
caggtatccggtaagcggcagggtcggaacaggagagcgcacgagggagcttccagggggaaacgcctggtatctttata
gtcctgtcgggtttcgccacctctgacttgagcgtcgatttttgtgatgctcgtcaggggggcggagcctatggaaaaacgcca
gcaacgcggcctttttacggttcctggccttttgctggccttttgctcacatgttctttcctgcgttatcccctgattctgtggataac
cgtattaccgcctttgagtgagctgataccgctcgccgcagccgaacgaccgagcgcagcgagtcagtgagcgaggaagc
ggaagagcgcccaatacgcaaaccgcctctccccgcgcgttggccgattcattaatgcagctggcacgacaggtttcccgac
tggaaagcgggcagtgagcgcaacgcaattaatgtgagttagctcactcattaggcaccccaggctttacactttatgcttccg
gctcgtatgttgtgtggaattgtgagcggataacaatttcacacaggaaacagctatgaccatgattacgccaagcgcgcaatt
aaccctcactaaagggaacaaaagctggagctgcaagcttaatgtagtcttatgcaatactcttgtagtcttgcaacatggtaac
gatgagttagcaacatgccttacaaggagagaaaaagcaccgtgcatgccgattggtggaagtaaggtggtacgatcgtgcc
ttattaggaaggcaacagacgggtctgacatggattggacgaaccactgaattgccgcattgcagagatattgtatttaagtgc
ctagctcgatacataaacgggtctctctggttagaccagatctgagcctgggagctctctggctaactagggaacccactgctt
aagcctcaataaagcttgccttgagtgcttcaagtagtgtgtgcccgtctgttgtgtgactctggtaactagagatccctcagacc
cttttagtcagtgtggaaaatctctagcagtggcgcccgaacagggacttgaaagcgaaagggaaaccagaggagctctctc
gacgcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaaaa
ttcggttaaggccagggggaaagaaaaaatataaattaaaacatatagtatgggcaagcagggagctagaacgattcgcagtt
aatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttaga
caagatagaggaagagcaaaacaaaagtaagaccaccgcacagcaagcggccgctgatcttcagacctggaggaggaga
tatgagggacaattggagaagtgaattatataaatataaagtagtaaaaattgaaccattaggagtagcacccaccaaggcaaa
gagaagagtggtgcagagagaaaaaagagcagtgggaataggagctttgttccttgggttcttgggagcagcaggaagcac
tatgggcgcagcgtcaatgacgctgacggtacaggccagacaattattgtctggtatagtgcagcagcagaacaatttgctga
gggctattgaggcgcaacagcatctgttgcaactcacagtctggggcatcaagcagctccaggcaagaatcctggctgtgga
aagatacctaaaggatcaacagctcctggggatttggggttgctctggaaaactcatttgcaccactgctgtgccttggaatgct
agttggagtaataaatctctggaacagatttggaatcacacgacctggatggagtgggacagagaaattaacaattacacaag
cttaatacactccttaattgaagaatcgcaaaaccagcaagaaaagaatgaacaagaattattggaattagataaatgggcaag
tttgtggaattggtttaacataacaaattggctgtggtatataaaattattcataatgatagtaggaggcttggtaggtttaagaata
gtttttgctgtactttctatagtgaatagagttaggcagggatattcaccattatcgtttcagacccacctcccaaccccgagggg
acccgacaggcccgaaggaatagaagaagaaggtggagagagagacagagacagatccattcgattagtgaacggatctc
gacggtatcgattagactgtagcccaggaatatggcagctagattgtacacatttagaaggaaaagttatcttggtagcagttca
tgtagccagtggatatatagaagcagaagtaattccagcagagacagggcaagaaacagcatacttcctcttaaaattagcag
gaagatggccagtaaaaacagtacatacagacaatggcagcaatttcaccagtactacagttaaggccgcctgttggtgggc
ggggatcaagcaggaatttggcattccctacaatccccaaagtcaaggagtaatagaatctatgaataaagaattaaagaaaat
tataggacaggtaagagatcaggctgaacatcttaagacagcagtacaaatggcagtattcatccacaattttaaaagaaaagg
ggggattggggggtacagtgcaggggaaagaatagtagacataatagcaacagacatacaaactaaagaattacaaaaaca
aattacaaaaattcaaaattttcgggtttattacagggacagcagagatccagtttggctgcatggcgcgtctcgaggcatgctc
tcccatcaataacaaattcagtgacatcagtttatgaatatatgaaatttgccaaagctctgtttagaccactgagtaactca
cagctaggtttcaacttttcctttctaggttgtcttgggtttattgtaagagagcattatgaagaaaaaaatagatcataaagct
tcttcaggaagctggtttcatatggtggtttagatttaaatagtgattgtctagcaccatttgaaatcagtgttcttgggggaga
ccagctgcgctgcactaccaacagcaaaagaagtgaatgggacagctctgaagtatttgaaagcaacagcaggatgg
tgtgagaacctgcctcacatgtagctgaccccttcctcacccctgccaacagtggtggcatatatcacaaatggcagtcag
gtctctgcactggcggatccaactgtgatcgaaagttttccaaaaataagttgtgtctgtattgaacatgaacagactttcttct
tgtcattattctctaacaatactgcataacaattatttgcatacatttgcattgcattaagtattctaagtaatctagagacgattt
aaagtatacgggaggatgtgtgtaggttgtatgcaaatactacaccattttctatcagagacttgagcatctgtggattttggt
atccaaggggctttctggaaccaatccctcaaggataccaagggatgaatgtaattgtacaggatatcgcattgttggaatt
ttatacttctttgtggaataaacctatagcacttaatagatagtacagactcattccattgtgcctgggttaaagagcccaatg
tatgctggatttagtaagatttgggccctcccaaccctcacgaccttctgtgaccccttagaggatgactgatttcttttggtgtt
cagagtcaatataattttctagcaccatctgaaatcggttataatgattggggaagagcaccatgatgctgactgctgagag
gaaatgtattggtgaccgttggggccatggacaagaactaagaaaacaaatgcaaagcaataatgcaaaggtgattttt
cttcttccagtttctaagttgaatttcactgacctgaattgcatgtggtctcgagggcgcgtcgtgaggctccggtgcccgtca
gtgggcagagcgcacatcgcccacagtccccgagaagttggggggaggggtcggcaattgaaccggtgcctagagaagg
tggcgcggggtaaactgggaaagtgatgtcgtgtactggctccgcctttttcccgaggggggggagaaccgtatataagtgc
agtagtcgccgtgaacgttctttttcgcaacgggtttgccgccagaacacaggtaagtgccgtgtgtggttcccgcgggcctg
gcctctttacgggttatggcccttgcgtgccttgaattacttccacctggctgcagtacgtgattcttgatcccgagcttcgggttg
gaagtgggtgggagagttcgaggccttgcgcttaaggagccccttcgcctcgtgcttgagttgaggcctggcctgggcgctg
gggccgccgcgtgcgaatctggtggcaccttcgcgcctgtctcgctgctttcgataagtctctagccatttaaaatttttgatgac
ctgctgcgacgctttttttctggcaagatagtcttgtaaatgcgggccaagatctgcacactggtatttcggtttttggggccgcg
ggcggcgacggggcccgtgcgtcccagcgcacatgttcggcgaggcggggcctgcgagcgcggccaccgagaatcgg
acgggggtagtctcaagctggccggcctgctctggtgcctggcctcgcgccgccgtgtatcgccccgccctgggcggcaag
gctggcccggtcggcaccagttgcgtgagcggaaagatggccgcttcccggccctgctgcagggagctcaaaatggagga
cgcggcgctcgggagagcggggggtgagtcacccacacaaaggaaaagggcctttccgtcctcagccgtcgcttcatgt
gactccactgagtaccgggcgccgtccaggcacctcgattagttctcgtgcttttggagtacgtcgtctttaggttggggggag
gggttttatgcgatggagtttccccacactgagtgggtggagactgaagttaggccagcttggcacttgatgtaattctccttgg
aatttgccctttttgagtttggatcttggttcattctcaagcctcagacagtggttcaaagtttttttcttccatttcaggtgtcgtgagc
tagctctagagccaccatggccttaccagtgaccgccttgctcctgccgctggccttgctgctccacgccgccaggccggac
atccagatgacacagactacatcctccctgtctgcctctctgggagacagagtcaccatcagttgcagggcaagtcaggacat
tagtaaatatttaaattggtatcagcagaaaccagatggaactgttaaactcctgatctaccatacatcaagattacactcaggag
tcccatcaaggttcagtggcagtgggtctggaacagattattctctcaccattagcaacctggagcaagaagatattgccactta
cttttgccaacagggtaatacgcttccgtacacgttcggaggggggaccaagctggagatcacaggtggcggtggctcggg
cggtggtgggtcgggtggcggcggatctgaggtgaaactgcaggagtcaggacctggcctggtggcgccctcacagagcc
tgtccgtcacatgcactgtctcaggggtctcattacccgactatggtgtaagctggattcgccagcctccacgaaagggtctgg
agtggctgggagtaatatggggtagtgaaaccacatactataattcagctctcaaatccagactgaccatcatcaaggacaact
ccaagagccaagttttcttaaaaatgaacagtctgcaaactgatgacacagccatttactactgtgccaaacattattactacggt
ggtagctatgctatggactactggggccaaggaacctcagtcaccgtctcctcaaccacgacgccagcgccgcgaccacca
acaccggcgcccaccatcgcgtcgcagcccctgtccctgcgcccagaggcgtgccggccagcggggggggcgcagtg
cacacgagggggctggacttcgcctgtgatatctacatctgggcgcccttggccgggacttgtggggtccttctcctgtcactg
gttatcaccctttactgcaaacggggcagaaagaaactcctgtatatattcaaacaaccatttatgagaccagtacaaactactc
aagaggaagatggctgtagctgccgatttccagaagaagaagaaggaggatgtgaactgagagtgaagttcagcaggagc
gcagacgcccccgcgtacaagcagggccagaaccagctctataacgagctcaatctaggacgaagagaggagtacgatgt
tttggacaagagacgtggccgggaccctgagatggggggaaagccgagaaggaagaaccctcaggaaggcctgtacaat
gaactgcagaaagataagatggcggaggcctacagtgagattgggatgaaaggcgagcgccggaggggcaaggggcac
gatggcctttaccagggtctcagtacagccaccaaggacacctacgacgcccttcacatgcaggccctgccccctcgctaag
tcgacaatcaacctctggattacaaaatttgtgaaagattgactggtattcttaactatgttgctccttttacgctatgtggatacgct
gctttaatgcctttgtatcatgctattgcttcccgtatggctttcattttctcctccttgtataaatcctggttgctgtctctttatgagga
gttgtggcccgttgtcaggcaacgtggcgtggtgtgcactgtgtttgctgacgcaacccccactggttggggcattgccacca
cctgtcagctcctttccgggactttcgctttccccctccctattgccacggcggaactcatcgccgcctgccttgcccgctgctg
gacaggggctcggctgttgggcactgacaattccgtggtgttgtcggggaagctgacgtcctttccttggctgctcgcctgtgtt
gccacctggattctgcgcgggacgtccttctgctacgtcccttcggccctcaatccagcggaccttccttcccgcggcctgctg
ccggctctgcggcctcttccgcgtcttcgccttcgccctcagacgagtcggatctccctttgggccgcctccccgcctggaatt
cgagctcggtacctttaagaccaatgacttacaaggcagctgtagatcttagccactttttaaaagaaaaggggggactggaa
gggctaattcactcccaacgaagacaagatctgctttttgcttgtactgggtctctctggttagaccagatctgagcctgggagct
ctctggctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagtagtagttcatgtcatcttattattcagtat
ttataacttgcaaagaaatgaatatcagagagtgagaggaacttgtttattgcagcttataatggttacaaataaagcaatagcat
cacaaatttcacaaataaagcatttttttcactgcattctagttgtggtttgtccaaactcatcaatgtatottatcatgtctggctctag
ctatcccgcccctaactccgcccagttccgcccattctccgccccatggctgactaattttttttatttatgcagaggccgaggcc
gcctcggcctctgagctattccagaagtagtgaggaggcttttttggaggcctagctagggacgtacccaattcgccctatagt
gagtcgtattacgcgcgctcactggccgtcgttttacaacgtcgtgactgggaaaaccctggcgttacccaacttaatcgccttg
cagcacatccccctttcgccagctggcgtaatagcgaagaggcccgcaccgatcgcccttcccaacagttgcgcagcctgaa
tggcgaatgggacgcgccctgtagcggcgcattaagcgcggcgggtgtggtggttacgcgcagcgtgaccgctacacttgc
cagcgccctagcgcccgctcctttcgctttcttcccttcctttctcgccacgttcgccggctttccccgtcaagctctaaatcggg
ggctccctttagggttccgatttagtgctttacggcacctcgaccccaaaaaacttgattagggtgatggttcacgtagtgggcc
atcgccctgatagacggtttttcgccctttgacgttggagtccacgttctttaatagtggactcttgttccaaactggaacaacact
caaccctatctcggtctattcttttgatttataagggattttgccgatttcggcctattggttaaaaaatgagctgatttaacaaaaatt
taacgcgaattttaacaaaatattaacgcttacaatttaggtggcacttttcggggaaatgtgcgcggaacccctatttgtttattttt
ctaaatacattcaaatatgtatccgctcatgagacaataaccctgataaatgcttcaataatattgaaaaaggaagagtatgagta
ttcaacatttccgtgtcgcccttattcccttttttgcggcattttgccttcctgtttttgctcacccagaaacgctggtgaaagtaaaa
gatgctgaagatcagttgg

In some embodiments, a subject CAR of the present invention comprises a CD19 binding domain and a transmembrane domain. In one embodiment, the CAR comprises a CD19 binding domain and a transmembrane domain, wherein the transmembrane domain comprises a CD8 hinge region. In one embodiment, the CAR comprises a CD19 binding domain and a transmembrane domain, wherein the transmembrane domain comprises a CD8a transmembrane domain. In one embodiment, the CAR comprises a CD19 binding domain and a transmembrane domain, wherein the transmembrane domain comprises a CD8 hinge region and a CD8a transmembrane domain.

Accordingly, the present invention provides a modified immune cell or precursor cell thereof, e.g., a modified T cell, a modified NK cell, a modified NKT cell, comprising a chimeric antigen receptor (CAR) having affinity for CD19 as described herein.

### Human Antibodies

It may be preferable that the antigen binding domains of the CAR comprise human antibodies or fragments thereof. Fully human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741: each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923:5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993): Jakobovits et al., Nature, 362:255-258 (1993): Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991): Vaughan et al., Nature Biotech., 14:309 (1996)). Phage display technology (McCafferty et al., Nature. 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats: for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies may also be generated by in vitro activated B cells (see, U.S. Pat. Nos. 5,567,610 and 5,229,275, each of which is incorporated herein by reference in its entirety). Human antibodies may also be generated in vitro using hybridoma techniques such as, but not limited to, that described by Roder et al. (Methods Enzymol., 121:140-167 (1986)).

### Nucleic Acids and Expression Vectors

The present invention provides nucleic acids encoding a CAR and a microRNA. In certain aspects, the invention provides a nucleic acid encoding a CAR having affinity for a tumor associated antigen (e.g. CD19) and a miR-29a microRNA. In certain aspects, the invention provides a nucleic acid encoding a CAR and a miR-29b1 microRNA. In certain aspects, the invention provides a nucleic acid encoding a CAR, a miR-29a microRNA, and a miR-29b1 microRNA. As described herein, a subject CAR comprises an antigen binding domain (e.g., CD19 binding domain), a transmembrane domain, and an intracellular domain. Accordingly, the present invention provides a nucleic acid encoding an antigen binding domain (e.g., CD19 binding domain), a transmembrane domain, an intracellular domain, and encoding a miR-29a microRNA.

In certain embodiments, the invention includes a nucleic acid encoding a CAR and a miR-29a. In certain embodiments, the miR-29a is encoded by a nucleotide sequence 80%, 85%, 90%, 95%, 96%, 97%, 98%, 99%, or 100% identical to any of SEQ ID NOs: 28, 29, 30, 31, 32, and 33.

In certain embodiments, the invention includes a nucleic acid encoding a CAR and a miR-29b1. In certain embodiments, the miR-29b1 is encoded by a nucleotide sequence 80%, 85%, 90%, 95%, 96%, 97%, 98%, 99%, or 100% identical to any of SEQ ID NOs: 34, 35, 36, 37, 38, and 39.

In certain embodiments, the invention includes a nucleic acid encoding a CAR, a miR-29a, and a miR-29b1. In certain embodiments, the miR-29a and miR-29b1 are encoded by a nucleotide sequence 80%, 85%, 90%, 95%, 96%, 97%, 98%, 99%, or 100% identical to SEQ ID NO: 27.

In an exemplary embodiment, a nucleic acid encoding a CD19 CAR, miR-29a, and miR-29b1 of the present invention is encoded by a nucleic acid sequence comprising the nucleotide sequence set forth in SEQ ID NO: 1.

In some embodiments, a nucleic acid of the present disclosure may be operably linked to a transcriptional control element, e.g., a promoter, and enhancer, etc. Suitable promoter and enhancer elements are known to those of skill in the art.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used: see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter: see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like: or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173 (1): 86-93: Alpuche-Aranda et al., Proc. Natl. Acad. Sci. USA (1992) 89 (21): 10079-83), a nirB promoter (Harborne et al. Mol. Micro. (1992) 6:2805-2813), and the like (see, e.g., Dunstan et al., Infect. Immun. (1999) 67:5133-5141: McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al., Infect. Immun. (2002) 70:1087-1096): an rpsM promoter (see, e.g., Valdivia and Falkow Mol. Microbiol. (1996). 22:367): a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162): an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and P Lambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator: in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the EF-1 alpha promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28: e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, MA), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is for the production of a transgenic polypeptide product that is released upon CAR signaling. See, e.g., Chmielewski and Abken, Expert Opin. Biol. Ther. (2015) 15 (8): 1145-1154; and Abken, Immunotherapy (2015) 7 (5): 535-544.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art: many are commercially available for generating a subject recombinant construct. The following vectors are provided by way of example, and should not be construed in any way as limiting: Bacterial: pBs, phagescript, PsiX174, pBluescript SK, pBs KS, pNH8a, pNH16a, pNH18a, pNH46a (Stratagene, La Jolla, Calif., USA): pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097: WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863: Jomary et al., Gene Ther. (1997) 4:683 690, Rolling et al., Hum. Gene Ther. (1999) 10:641-648: Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., murine leukemia virus, spleen necrosis virus, and vectors derived from retroviruses such as Rous sarcoma virus, Harvey sarcoma virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584: WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding a CAR. In some embodiments, the expression vector (e.g., lentiviral vector) will comprise additional elements that will aid in the functional expression of the CAR encoded therein. In some embodiments, an expression vector comprising a nucleic acid encoding a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-la promoter) may be less likely to induce integration mediated genotoxicity, and may abrogate the ability of the retroviral vector to transform stem cells Other physiological promoters suitable for use in a vector (e.g., a lentiviral vector) are known to those of skill in the art and may be incorporated into a vector of the present invention. In some embodiments, the vector (e.g., a lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., a lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art: any known method can be used to synthesize RNA comprising a sequence encoding a CAR of the present disclosure. Methods for introducing RNA into a host cell are known in the art. See, e.g., Zhao et al. Cancer Res. (2010) 15:9053. Introducing RNA comprising a nucleotide sequence encoding a CAR of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82).

### Methods of Generating Modified Immune Cells

The present invention provides methods for producing/generating a modified immune cell or precursor cell thereof (e.g., a T cell/NK cell/NKT cell). The cells are generally engineered by introducing a nucleic acid encoding a subject CAR (e.g., CD19 CAR).

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12 (8): 861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR of the invention is introduced into a cell by an expression vector. Expression vectors comprising a nucleic acid encoding a subject CAR (e.g., CD19 CAR) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggy back, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7 (20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

The present invention also provides genetically engineered cells which include and stably express a subject CAR of the present disclosure. In some embodiments, the genetically engineered cells are genetically engineered T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In one embodiment, the genetically engineered cells are autologous cells.

Modified cells (e.g., comprising a subject CAR) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO: dicetyl phosphate (“DCP”) can be obtained from K & K Laboratories (Plainview, NY): cholesterol (“Choi”) can be obtained from Calbiochem-Behring: dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR: “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### RNA

In one embodiment, the nucleic acids introduced into the host cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985): Nacheva and Berzal-Herranz, Eur. J. Biochem., 270:1485-65 (2003).

The conventional method of integration of poly A/T stretches into a DNA template is molecular cloning. However, poly A/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The poly A/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli poly A polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In certain exemplary embodiments, RNAs produced by the methods disclosed herein include a 5′ cap. The 5′ cap is provided using techniques known in the art and described herein (Cougot, et al., Trends in Biochem. Sci., 29:436-444 (2001): Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The disclosed methods can be applied to the modulation of host cell activity in basic research and therapy, in the fields of cancer, stem cells, acute and chronic infections, and autoimmune diseases, including the assessment of the ability of the genetically modified host cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of host cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this non-physiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatuses for therapeutic application of electroporation are available commercially, e.g., the MedPulser™ DNA Electroporation Therapy System (Inovio/Genetronics, San Diego, CA), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

Accordingly, the present invention provides a method for generating a modified immune cell or precursor cell thereof comprising introducing into the cell an isolated nucleic acid (e.g., an expression construct) encoding for a subject CAR as described herein, using any of the delivery methods described herein or are known to those of skill in the art.

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells and/or NKT cells. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (T_{SCM}), central memory T (TCM), effector memory T (T_{EM}), or terminally differentiated effector memory T cells, tumor-infiltrating lymphocytes (T_{IL}), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow; thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some certain, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker⁻) or express relatively low levels (marker^{low}) of one or more markers. For example, in certain aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In certain exemplary embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations. In some embodiments, combining TCM-enriched CD8+ T cells and CD4+ T cells further enhances efficacy.

In some embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and/or a CD8+T population is enriched for central memory (TCM) cells. In some embodiments, the enrichment for central memory T (TCM) cells is based on positive or high surface expression of CD45RO, CD62L, CCR7, CD28, CD3, and/or CD 127; in certain aspects, it is based on negative selection for cells expressing or highly expressing CD45RA and/or granzyme B. In certain aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in certain aspects are carried out simultaneously and in other aspects are carried out sequentially, in either order. In some certain, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of Immune Cells

Whether prior to or after modification of cells to express a subject CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30 (8): 3975-3977: Haanen et al., J. Exp. Med. (1999) 190 (9): 1319-1328; and Garland et al., J. Immunol. Methods (1999) 227 (1-2): 53-63).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

### Methods of Treatment

The modified cells (e.g., miR-29a-expressing T cells) described herein, may be included in a composition for immunotherapy. The composition may include a pharmaceutical composition and further include a pharmaceutically acceptable carrier. A therapeutically effective amount of the pharmaceutical composition comprising the modified cells may be administered.

In one aspect, the invention includes a method of enhancing an immune response to an immunotherapy in a subject in need thereof, the method comprising contacting one or more immune effector cells of the subject with a nucleic acid vector encoding a miR-29a microRNA prior to treatment with the immunotherapy such that the immune effector cells express a high level of miR-29a microRNA as compared to uncontacted immune effector cells, thereby enhancing the effect of the immunotherapy. In certain embodiments, the immune effector cells are T cells. In certain preferred embodiments, the T cells are CD8+ T cells. In certain embodiments, the relatively elevated level of miR-29a renders the immune effectors cells resistant to immune exhaustion. In certain embodiments the immune response to an immunotherapy may be associated with a vaccine, a treatment with an immune-modulating agent, an adoptive transfer of immune-modulating cells, or any combination thereof. Here, the resistance to immune exhaustion enhances the cytotoxic function of the immune effectors and prolongs their persistence in vivo.

In one aspect, the invention includes a method of treating a chronic infection in a subject in need thereof, the method comprising contacting one or more immune effector cells of the subject with a nucleic acid vector encoding a miR-29a microRNA such that the immune effector cells express a high level of miR-29a microRNA as compared to uncontacted immune effector cells, thereby treating the chronic infection. In certain embodiments, the immune effector cells are T cells. In certain preferred embodiments, the T cells are CD8+ T cells. In certain embodiments, the chronic infections treated by the methods of the invention can be caused by bacterial, viruses, or parasites. Examples of persistent or chronic infection include, but are not limited to, herpesviruses, retroviruses, papillomaviruses, hepatitis viruses, human immunodeficiency virus (HIV), Varicella-zoster virus, measles virus, and human cytomegalovirus and the like. Examples of human parasites that cause chronic disease due in part to T cell immune exhaustion include, but are not limited to Toxoplasma gondii, Plasmodium sp., and Leishmania sp.

In one aspect, the invention includes a method of treating a tumor antigen associated cancer in a subject in need thereof. In another aspect, the invention includes a method of treating a tumor antigen-associated cancer in a subject comprising administering to a subject in need thereof a therapeutically effective population of modified immune cells of the present invention.

The method comprises administering to the subject a modified immune cell (e.g., tumor associated antigen specific CAR T cell) of the present invention.

As used herein, the terms “subject” and “patient” refer to organisms to be treated by the methods of the present invention. The terms “subject” and “patient” may be used interchangeably herein. Such organisms include, but are not limited to, mammals (e.g., murines, simians, equines, bovines, porcines, canines, felines, and the like), and in an exemplary embodiment includes humans. As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced tumor burden, reduced rate of cancer cell infiltration into peripheral organs, or reduced rate of tumor metastasis or tumor growth.

“Tumor” as it applies to a subject diagnosed with, or suspected of having, cancer, refers to a malignant or potentially malignant neoplasm or tissue mass of any size.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

In one aspect, the invention includes a method of treating a tumor antigen-associated cancer in a subject in need thereof, comprising administering to the subject a therapeutically effective population of modified immune cells, wherein the modified immune cells comprise a chimeric antigen receptor (CAR). In certain embodiments, the CAR comprises a CD19-specific antigen binding domain, a transmembrane domain, a costimulatory signaling domain, an intracellular signaling domain, and a domain encoding a miR-29a.

A CAR of the present disclosure, when present in a T lymphocyte, can mediate cytotoxicity toward a target cell. A CAR of the present disclosure binds to an antigen present on a target cell, thereby mediating killing of a target cell by a T lymphocyte or an NK cell genetically modified to produce the CAR. The domain encoding the miR-29a microRNA of the CAR further enables the T lymphocyte to resist the development of exhaustion, thereby prolonging cytotoxic function. The antigen-binding domain of the CAR (e.g., anti-CD19 scFv) binds to an antigen present on the surface of a target cell (e.g., CD19 antigen). Target cells include, but are not limited to, cancer cells. Thus, the present disclosure provides methods of killing, or inhibiting the growth of, a target cancer cell, the method involving contacting a cytotoxic immune effector cell (e.g., a cytotoxic T cell) that is genetically modified to produce a subject CAR, such that the T lymphocyte recognizes an antigen present on the surface of a target cancer cell, and mediates killing of the target cell.

The present disclosure provides a method of treating cancer in a subject having a cancer, the method comprising: i) introducing a chimeric antigen receptor of the present disclosure, or introducing an expression vector of the present disclosure, into a cell, to produce a modified cell; and ii) administering the modified cell to the subject. In some embodiments, the cell is obtained from the subject (i.e., the cell is autologous), engineered ex vivo, and administered to the same subject. In some embodiments, the cell is obtained from one subject, engineered ex vivo, and administered to a second suitable subject (i.e., the cell is allogeneic).

In some embodiments, a method is provided including retrieving cytotoxic cells from a subject, genetically modifying the cytotoxic cells by introducing a CAR gene of the present invention into the cytotoxic cells, and administering the modified cytotoxic cells to the subject. In some embodiments, the cytotoxic cells are selected from T cells, naive T cells, memory T cells, effector T cells, natural killer cells, and macrophages. In one embodiment, the cytotoxic cells are T cells. In a preferred embodiment, the cytotoxic cells are CD8+ T cells.

In one embodiment, the T cells are obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of T cell lines available in the art, may be used. In some embodiments of the present invention, T cells can be obtained from blood collected from a subject using any number of techniques known to the skilled artisan, such as Ficoll™ separation.

For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In one embodiment, the time period ranges from 30 minutes to 36 hours or longer and all integer values there between. In one embodiment, the time period is at least 1, 2, 3, 4, 5, or 6 hours. In one embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled person would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

The obtained cells are then modified as described herein. A polynucleotide encoding the subject CAR (e.g., a CD19/miR-29a CAR), typically located in an expression vector, is introduced into the cytotoxic cells such that the cytotoxic cells will express, preferably stably, the CAR. In some embodiments, the polynucleotide encoding the CAR also encodes a CAR inducible expression cassette for a transgenic polypeptide product that is produced and released upon CAR signaling. In some embodiments, the polynucleotide encoding the CAR also encodes a cytokine (e.g., IL-12) operably linked to a T-cell activation responsive promoter. In some embodiments, the expression vector comprises both the polynucleotide encoding the CAR and the polynucleotide encoding the cytokine operably linked to the T-cell activation responsive promoter. See, e.g., Chmielewski and Abken, Expert Opin. Biol. Ther. (2015) 15 (8): 1145-1154; and Abken, Immunotherapy (2015) 7 (5): 535-544. In some embodiments the cells are genetically engineered with an expression vector comprising the polynucleotide encoding the CAR and an expression vector comprising the polynucleotide encoding the cytokine (e.g., IL-12) operably linked to the T-cell activation responsive promoter. In some embodiments, the polynucleotide introduction need not result in integration but rather only transient maintenance of the polynucleotide introduced may be sufficient. In this way, one could have a short term effect, where cytotoxic cells could be introduced into the host and then turned on after a predetermined time, for example, after the cells have been able to migrate to a particular site for treatment.

Depending upon the nature of the cytotoxic cells and the diseases to be treated, the modified cytotoxic cells (e.g., modified T cells) may be introduced into the subject, e.g. a mammal, in a wide variety of ways. The genetically engineered cytotoxic cells may be introduced at the site of the tumor. In one embodiment, the genetically engineered cytotoxic cells navigate to the cancer or are modified to navigate to the cancer. The number of modified cytotoxic cells that are employed will depend upon a number of factors such as the circumstances, the purpose for the introduction, the lifetime of the cells, the protocol to be used. For example, the number of modified cytotoxic cells that are employed may depend upon the number of administrations, the ability of the cells to multiply, and the stability of the recombinant construct. The modified cytotoxic cells may be applied as a dispersion injected at or near the site of interest. In one embodiment, the cells may be in a physiologically-acceptable medium.

It should be appreciated that the treatment method is subject to many variables, such as the cellular response to the CAR, the efficiency of expression of the CAR by the cytotoxic cells and, as appropriate, the level of secretion, the activity of the expressed CAR, the particular need of the subject, which may vary with time and circumstances, the rate of loss of the cellular activity as a result of loss of modified cytotoxic cells or the expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

In an exemplary embodiment, provided is a method of treating a CD19 associated cancer in a subject in need thereof, comprising administering to the subject a therapeutically effective composition comprising a modified T cell comprising: a tumor associated antigen-specific antigen binding; optionally a CD8a hinge domain; a CD8a transmembrane domain; a 4-1BB costimulatory signaling domain; a CD3 zeta intracellular signaling domain; and a domain encoding a miR-29a microRNA.

In certain embodiments, the population of modified immune cells administered to the subject comprises immune cells selected from the group consisting of natural killer (NK) cells, NKT cells, and T cells. In certain exemplary embodiments, the population of modified immune cells comprises modified T cells. In certain embodiments, the modified T cells are autologous.

The cells of the invention may be administered by any means known to one of ordinary skill in the art. For example, in certain embodiments, the administering may be performed via intratumoral delivery, via intravenous delivery, or via intraperitoneal delivery.

The amount of modified immune cells (e.g., modified T cells) to be administered to a subject in need is, generally, a therapeutically effective amount. As used herein, a “therapeutically effective amount” refers to a dose of modified immune cells that results in the cytotoxic killing of cancer cells in the subject. In some embodiments, a suitable dose of modified immune cells when administered to the subject, results in a reduction of the cancer. Reduction of the cancer can be in the form of an output of one or more parameters indicative of the cancer, and be performed by various methods known in the art, for example, by detection of circulating tumor cells, detection of certain cancer-specific markers in the blood, detection of markers in a biopsy, tumor imaging, and the like. Generally, a suitable dose of modified immune cells when administered to the subject, results in a reduction of one or more parameters at least about 5%, 10%, 15%, 20%, 25%, 30%, 35%, 40%, 45%, 50%, 55%, 60%, 65%, 70%, 75%, or more from baseline.

As used herein, the term “effective amount” or “therapeutically effective amount” refers to the amount of an agent (e.g., a tumor associated antigen specific CAR T cell composition) sufficient to effect beneficial or desired results. An effective amount can be administered in one or more administrations, applications or dosages and is not intended to be limited to a particular formulation or administration route. A therapeutically effective amount can vary depending upon known factors, such as the mode and route of administration: the age, health, and weight of the recipient: the type and extent of disease or indication to be treated, the nature and extent of symptoms, kind of concurrent treatment, frequency of treatment, and the effect desired.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in certain aspects include a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In certain aspects, the desired dose is a desired number of cells or a desired number of cells per unit of body weight of the subject to whom the cells are administered, e.g., cells/kg. In certain aspects, the desired dose is at or above a minimum number of cells or minimum number of cells per unit of body weight. In certain aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In certain aspects, the desired dose is a desired number of cells of the sub-type or population, or a desired number of such cells per unit of body weight of the subject to whom the cells are administered, e.g., cells/kg. In certain aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg, 10⁴, and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells. In an exemplary embodiment, a suitable dosage is from about 1×10⁷ total cells to about 3×10⁷ total cells.

In some embodiments, the dose of total cells and/or dose of individual sub-populations of cells is within a range of between at or about 1×10⁵ cells/m² to about 1×10¹¹ cells/m². In an exemplary embodiment, the dose of total cells and/or dose of individual sub-populations of cells is within a range of between at or about 1×10⁷/m² to at or about 3×10⁷/m². In an exemplary embodiment, the dose of total cells and/or dose of individual sub-populations of cells is within a range of between at or about 1×10⁸/m² to at or about 3×10⁸/m². In some embodiments, the dose of total cells and/or dose of individual sub-populations of cells is the maximum tolerated dose by a given patient.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion. In some embodiments, a dose of modified cells is administered to a subject in need thereof, in a fractionated dose or split dose. In such embodiments, the first dose is administered, and a subsequent dose is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, 2 or more weeks, 3 or more weeks, 4 or more weeks, 5 or more weeks, or any period in between, after the first dose.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells is measured by assaying expression and/or secretion of one or more cytokines, such as CD 107a, IFNγ, IL-2, and TNF. In certain aspects, the biological activity is measured by assessing clinical outcome, such as reduction in tumor burden or load.

In certain embodiments, the subject can be administered, in addition to the CAR, a secondary treatment.

In some embodiments, the subject can be administered conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In certain exemplary embodiments, the conditioning therapy comprises administering an effective amount of a combination of cyclophosphamide and fludarabine to the subject. Accordingly, the present disclosure provides a method of treatment comprising administering a conditioning therapy comprising an effective amount of a combination of cyclophosphamide and fludarabine to the subject, prior to administering CAR T therapy. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In certain embodiments, the subject is provided a secondary treatment. Secondary treatments include but are not limited to chemotherapy, radiation, surgery, and medications.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days,

Dosing of lymphodepletion chemotherapy may be scheduled on Days −6 to −4 (with a −1 day window, i.e., dosing on Days −7 to −5) relative to CAR-T infusion on day 0.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS) (grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability: with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose): tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47: Teachey et al. (2016) Cancer Discov, 6 (6): 664-679).

Features consistent with macrophage activation syndrome (MAS) or hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

### Pharmaceutical Compositions

The modified immune cell (e.g., a tumor associated antigen specific CAR T cell) described herein may be included in a composition for immunotherapy, in particular for treating a tumor antigen-associated cancer (e.g. CD19 associated cancer). The composition may include a pharmaceutical composition and further include a pharmaceutically acceptable carrier. A therapeutically effective amount of the pharmaceutical composition comprising the modified immune cells may be administered.

Pharmaceutical compositions of the present invention may comprise the modified immune cell as described herein, in combination with one or more pharmaceutically or physiologically acceptable carriers, diluents or excipients. Such compositions may comprise buffers such as neutral buffered saline, phosphate buffered saline and the like: carbohydrates such as glucose, mannose, sucrose or dextrans, mannitol: proteins: polypeptides or amino acids such as glycine: antioxidants: chelating agents such as EDTA or glutathione: adjuvants (e.g., aluminum hydroxide); and preservatives. In certain exemplary embodiments, compositions described herein are formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

Also provided are populations of immune cells described herein, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as regulatory T cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In certain aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In certain aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980).

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in certain aspects are included in the compositions. Suitable buffering agents include, for example, citric acid, sodium citrate, phosphoric acid, potassium phosphate, and various other acids and salts. In certain aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins: 21st ed. (May 1, 2005).

Formulations described herein can include aqueous solutions. The formulation or composition may also contain more than one active ingredient useful for the particular indication, disease, or condition being treated with the cells, e.g., those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in certain aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in certain aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

It can generally be stated that a pharmaceutical composition comprising the modified immune cells described herein may be administered at a dosage of 10⁴ to 10⁹ cells/kg body weight, in some instances 10⁵ to 10⁶ cells/kg body weight, including all integer values within those ranges. Immune cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified immune cells of the invention may be administered by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal. In some embodiments, the administration of the modified immune cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984): “Culture of Animal Cells” (Freshney, 2010): “Methods in Enzymology” “Handbook of Experimental Immunology” (Weir, 1997); “Gene Transfer Vectors for Mammalian Cells” (Miller and Calos, 1987): “Short Protocols in Molecular Biology” (Ausubel, 2002): “Polymerase Chain Reaction: Principles, Applications and Troubleshooting”, (Babar, 2011): “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Mice: Six to eight week-old C57BL/6 Ly5.2CR (CD45.1) and C57BL/6 (CD45.2) mice were purchased from NCI. Both male and female mice were used. P14 TCR transgenic mice expressing a TCR specific for the LCMV D^{b}gp33-41 epitope were bred in house. All mice were used in accordance with Institutional Animal Care and Use Committee guidelines for the University of Pennsylvania.

Viral infections: Mice were infected intraperitoneally (i.p.) with 2×10⁵ plaque-forming units (PFU) LCMV Armstrong or intravenously (i.v.) via tail-vein injection with 4×10⁶ PFU LCMV Cl-13 or 2×10⁴ PFU LCMV V35A. Recombinant influenza virus (H1N1) expressing the LCMV gp33-41 epitope (PR8-GP33) was obtained from Dr. Richard J. Webby (St. Jude Children's Research Hospital, Memphis, TN).

Retroviral vector (RV) experiments: The miR-29a (MI0000576) cDNA clone was obtained from OriGene. MiR-29a cDNA was cloned into the MSCV-IRES-VEX plasmid. RV was produced in 293T cells with MSCV and pCL-Eco plasmids using Lipofectamine 3000. RV transduction was performed as described. Briefly, purified CD8⁺ T cells were stimulated with 100 U/mL recombinant human IL-2, 1 μg/mL anti-mouse CD38, and 0.5 μg/mL anti-mouse CD28. After 18-24 hrs of stimulation, cells were transduced in the presence of polybrene (0.5 μg/ml) during spin infection (2,000 g for 60 min at 32° C.) following incubation at 37° C. for 6 hrs.

Cell preparation, flow cytometry and cell sorting: Spleens were mechanically disrupted onto a 70 μM cell strainer and red blood cell were lyzed with ACK buffer (Gibco). Cells were stained with extracellular antibodies for 30 min on ice. For transcription factor detection, cells were fixed and permeabilized using the Foxp3 Transcription Factor buffer set (ThermoFisher Scientific). Samples were acquired on an LSR II and analysed with FlowJo v. 10 software (Tree Star Inc).

For cell sorting, CD8⁺ T cells were enriched using the Easy Sep^{tm} CD8⁺ T cell isolation Kit (StemCell) and VEX+ cells were sorted based on CD8, CD45.1, CD45.2 and VEX on an BD FACSARIA (BD Bioscience) using a 70 micron nozzle.

Intracellular cytokine staining: Splenocytes (1-2×10⁶) were re-stimulated in vitro for 5 h at 37° C. in RPMI supplemented with GolgiStop (1/250; BD bioscience), GolgiPlug (1/500): BD bioscience), gp₃₃₋₄₁ peptide (NIH, 0.4 μg/ml) and CD107a antibodies (1/500). Cells were then washed and stained using the BD Fixation/permeabilization kit (BD Bioscience).

Microarrays processing and analysis: Sorted LCMV D^{b} gp-33 specific and naïve CD8 T cells were resuspended in TRIzol (Thermo Fisher Scientific). RNA was isolated using the RNeasy Micro Kit (Qiagen). Microarray microRNA 2.0 (Affymetrix) was performed at the Penn Microarray Facility. Cel files from the Microarrays were read in R using the ReadAffy function from affy package and the counts were quantile normalized using the NormiR function from ExiMiR package. (https://www.rdocumentation.org/packages/ExiMiR/versions/2.14.0/topics/NormiR). R package limma was used to fit the counts data to a model based on groups.

RNA isolation and sequencing: RNA was isolated using the RNeasy Micro Kit (Qiagen), according to the manufacturer's instructions. Quality control analysis, library generation, and RNA-seq were carried out by the Oncogenomics Core Facility at the University of Miami. RNA-Seq libraries were prepared using Roche Kapa RNA HyperPrep with Riboerase. RNA sample RIN was equal to 10. Input amounts were split into 2 batches, low input 11.2 ng and standard input 30 ng. Library amplification cycles were 14 for low input and 12 for standard input. Libraries were cleaned using standard AMPure bead protocols and balanced using fragment analysis (Agilent 5200) and DNA quantitation (Qubit). The library pool was sequenced on an Illumina NovaSeq 6000 on an S2 flow cell as 2×150 bp reads. Basecalling and demultiplexing was performed in BaseSpace using default bcl2fastq parameters. Raw paired-ended FASTQ data were assessed for quality with FastQC (ver. 11.5, Andrews, S. 2010). Trimmomatic (ver.0.32) was then used to remove adapters, platform-specific sequences, and low-quality leading and trailing bases from reads (50). Then STAR (ver.2.5.0) was utilized to map reads to the reference genome GRCm38 (51). The mapped data were assigned with genomic features with featureCounts ver. 1.5.0 (52). Fold changes of differential expression were estimated through DESeq2 (53).

Network analysis and GSEA: Network analysis was performed with Ingenuity Pathway Analysis. Differential miRNAs were used as input to the MicroRNA Target Filter in IPA (Qiagen https://digitalinsights.qiagen.com/products-overview/discovery-insights-portfolio/analysis-and-visualization/qiagen-ipa/features/microrna-target-filter/) along with differential mRNAs from a previous study (5) to examine the microRNA-mRNA pairings. GSEA was performed using MSigDb (v5.1) from the Broad Institute.

Statistical analysis: Samples were tested for normal distribution using D'Agostino and Pearson omnibus normality test. For samples that passed normality test, statistical significance was calculated using unpaired two-tailed student's t-test (for n=2) or one-way ANOVA with Bonferroni multiple comparisons post-test (for n>2). For samples that did not pass normal distribution, statistical significance was calculated using non-parametric Mann-Whitney test (for n=2) or Kruskal-Wallis test with Dunn's multiple comparisons post-test (for n>2). Statistical significance was calculated by Prism 5 (GraphPad Software). P values are reported in the Fig. legends.

### Example 1: MicroRNAs are Differentially Regulated in CD8 T Cells Responding to Acutely Resolved or Chronic Viral Infection

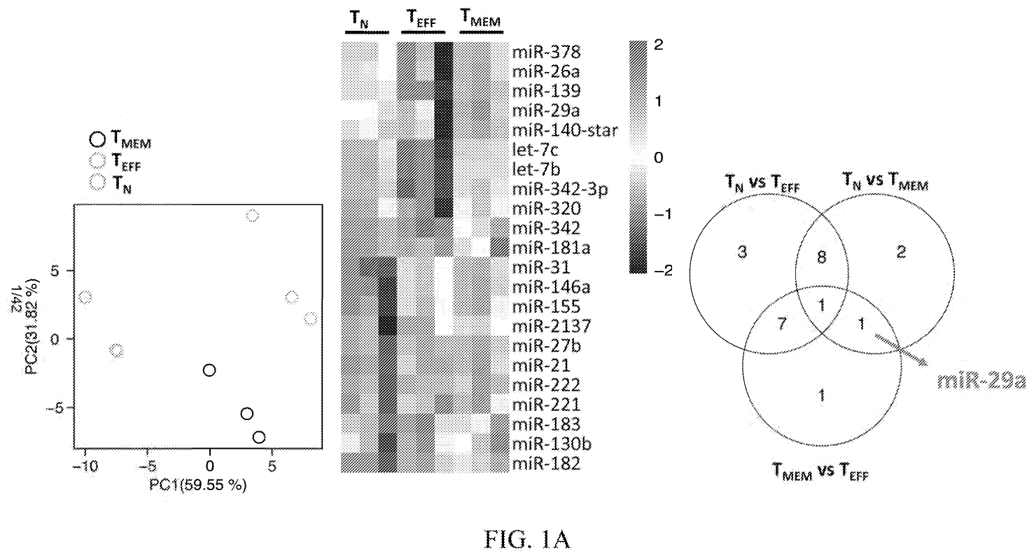

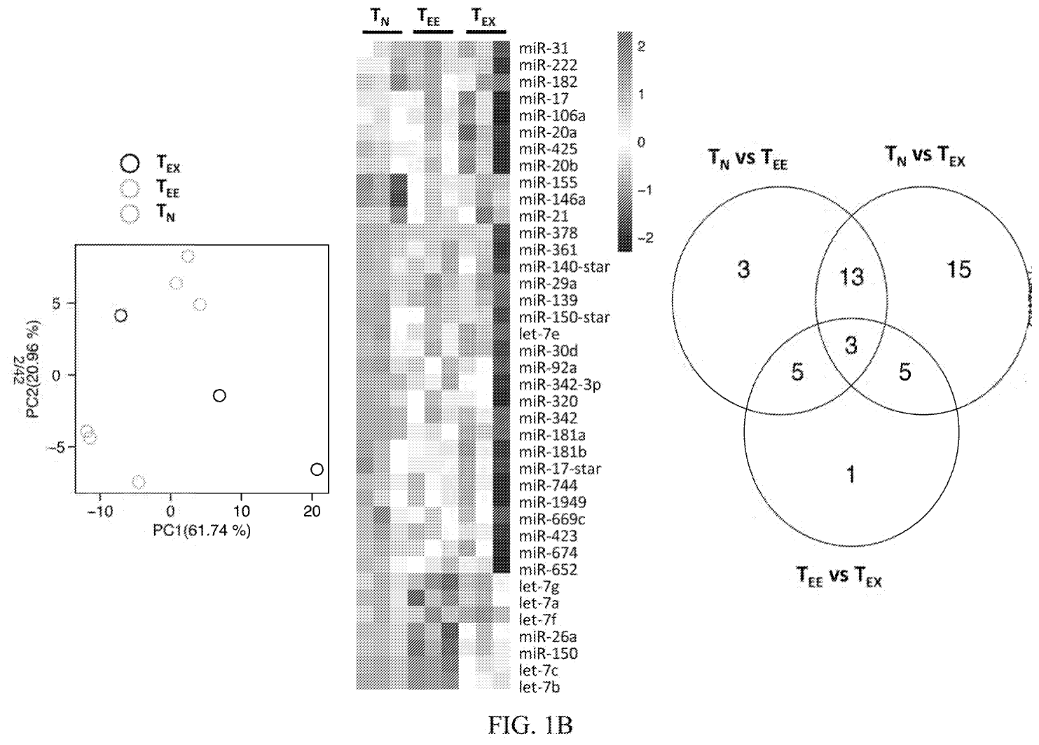

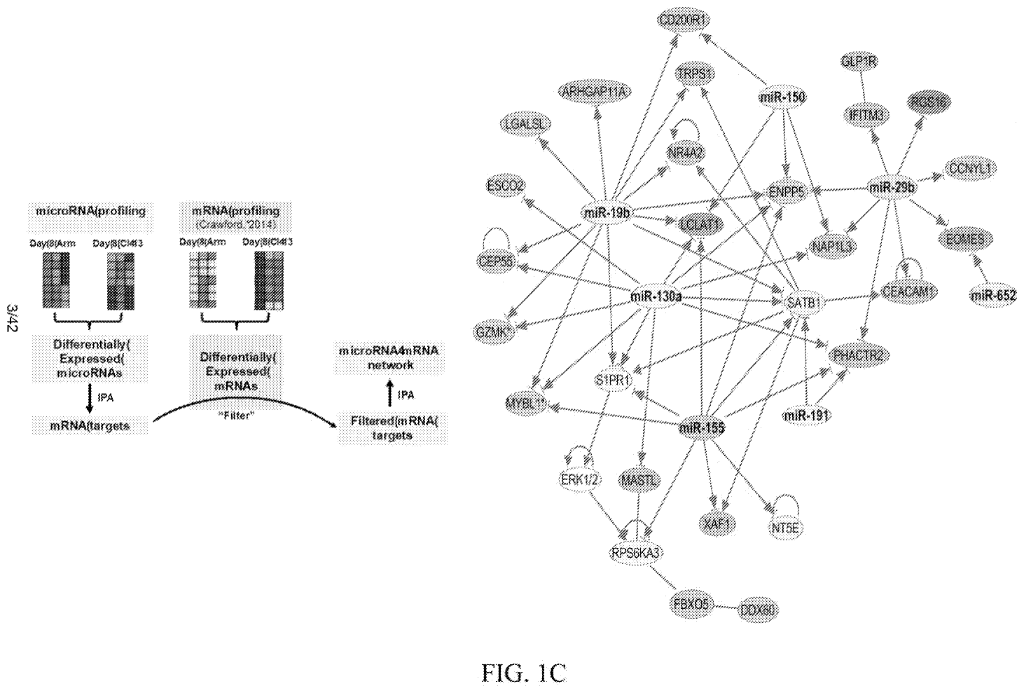

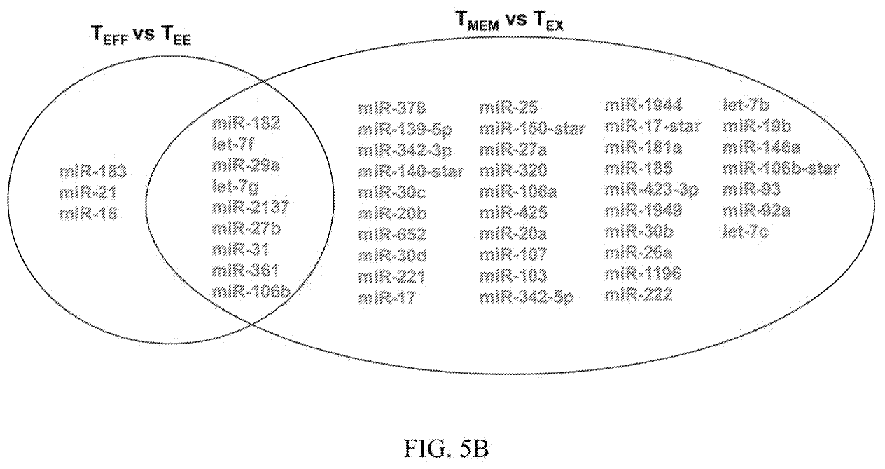

Global miR expression profiles were investigated in virus-specific CD8 T cells following acutely resolved or chronic infection of mice with the Armstrong (Arm) or clone 13 strains of lymphocytic choriomeningitis virus (LCMV), respectively. LCMV D^{b}GP3341 specific effector (T_{EFF}) and memory (T_{MEM}) CD8 T cells were isolated from mice infected with LCMV Arm at day 8 (d8) or d30 post-infection (p.i.), respectively. As a control, naïve CD8 T cells (T_{N}) were isolated from uninfected mice. Studies then examined the miR expression of isolated LCMV D^{b}GP33-41 specific CD8 T cells using Affymetrix miR arrays. Principal Component Analysis (PCA) demonstrated that miR expression patterns were distinct in T_{N}, T_{EFF} and T_{MEM} CD8 T cells (FIG. 1A). Among miRs differentially expressed (DEM; FDR<0.05) between T_{N}, T_{EFF} and T_{MEM} Were several previously shown to regulate T cell differentiation, including miR-155, miR-146a, let-7b and let-7c (FIG. 1A, FIG. 5A). It was asked whether any individual miRs were distinctly expressed by T_{MEM}. Indeed, miR-29a was the only miR uniquely expressed by T_{MEM} (i.e. DE in T_{MEM} VS Tx and T_{MEM} VS T_{EFF} but not in T_{N} VS T_{EFF}: FIG. 1A, FIG. 5A) suggesting a potential role for miR-29a in the biology of T_{MEM}.

During chronic LCMV infection optimal T_{EFF} and T_{MEM} do not develop and instead virus-specific CD8 T cells become exhausted. Therefore, miR expression was next examined in virus-specific CD8 T cells at d8 (“T_{EE}”; “early exhausted”) and at d30 (T_{EX}) p.i. with LCMV clone 13. Similar to acute infection, PCA analysis revealed distinct miR profiles in virus-specific CD8 T cells during chronic infection (FIG. 1B). Comparison of miR expression between virus-specific CD8 T cells from acute versus chronic infection identified 12 DEM at d8 p.i. (T_{EFF} Versus T_{EE}) and 46 DEM at d30 p.i. (T_{MEM} versus T_{EX}), indicating that miR expression patterns diverge as CD8 T cell differentiation patterns become more distinct over time following acutely resolved versus chronic viral infection (FIG. S1B). All DEM at d30 p.i., including miR-29a, were downregulated in T_{EX} compared to T_{MEM}, suggesting that failure to upregulate or sustain specific miR expression during chronic infection may contribute to CD8 T cell exhaustion. Thus, these analyses identified distinct patterns of miR expression in CD8 T cells and revealed miR-29a as a T_{MEM} specific miR that is notably absent from T_{EX}.

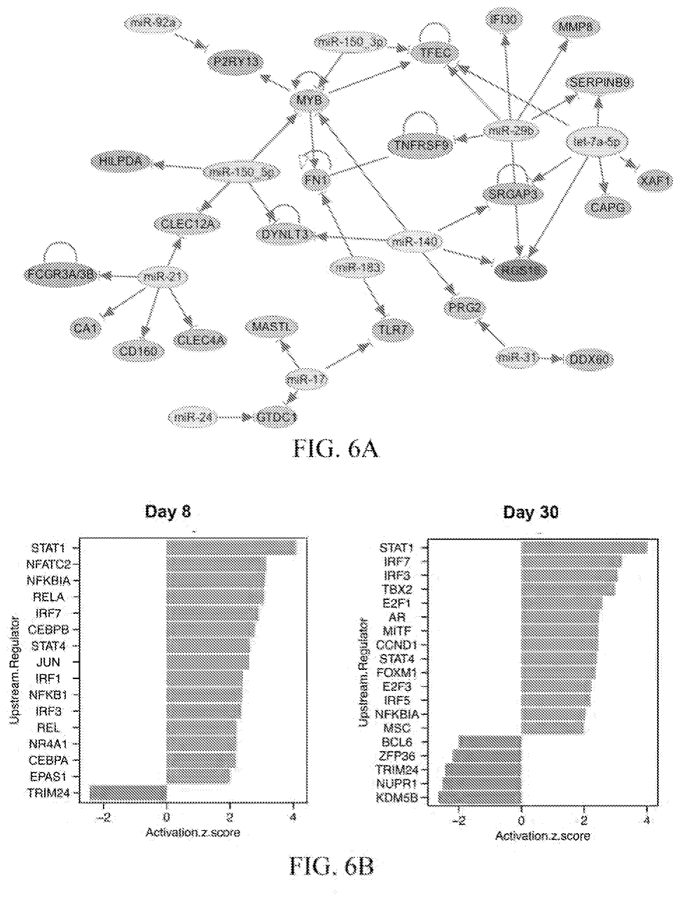

Without wishing to be bound by theory, it was then hypothesized that the effects of miRs in CD8 T cell differentiation were due to effects on complementary target mRNAs. Studies were therefore performed in which an integrated analysis of miR expression patterns was compared with transcriptional profiles of mRNA for these cell types. miR expression patterns (DEM P<0.05) between acute and chronic infection at d8 and d30 p.i. were used to generate a list of predicted miR target mRNAs at each timepoint. This list was then cross-referenced with differentially expressed genes expressed at these time points (DEG: P<0.05). Since miRs function typically by inducing mRNA degradation, studies then examined DEG that were expressed in the opposite direction of their predicted targeting miR. A network constructed using these miR-mRNA data revealed several miR nodes regulating key genes, including some previously shown to regulate CD8 T cell biology (miR-150 and miR-155), but also identified several novel miRs, including miR-29a, miR-19b, miR-130a and associated mRNA targets (FIG. 1C and FIG. 6A). Upstream regulators of this miR-mRNA network were central to the cellular response to inflammation (STATs, IRFs, NFKB1) and TCR signaling (NFATC2, NR4A1: FIG. S2B) suggesting that this set of miRs may function as a rheostat, limiting CD8 T cell responses to inflammatory and/or antigen signaling and that lower expression of these miRs in T_{EX} may contribute to chronic overstimulation and exhaustion.

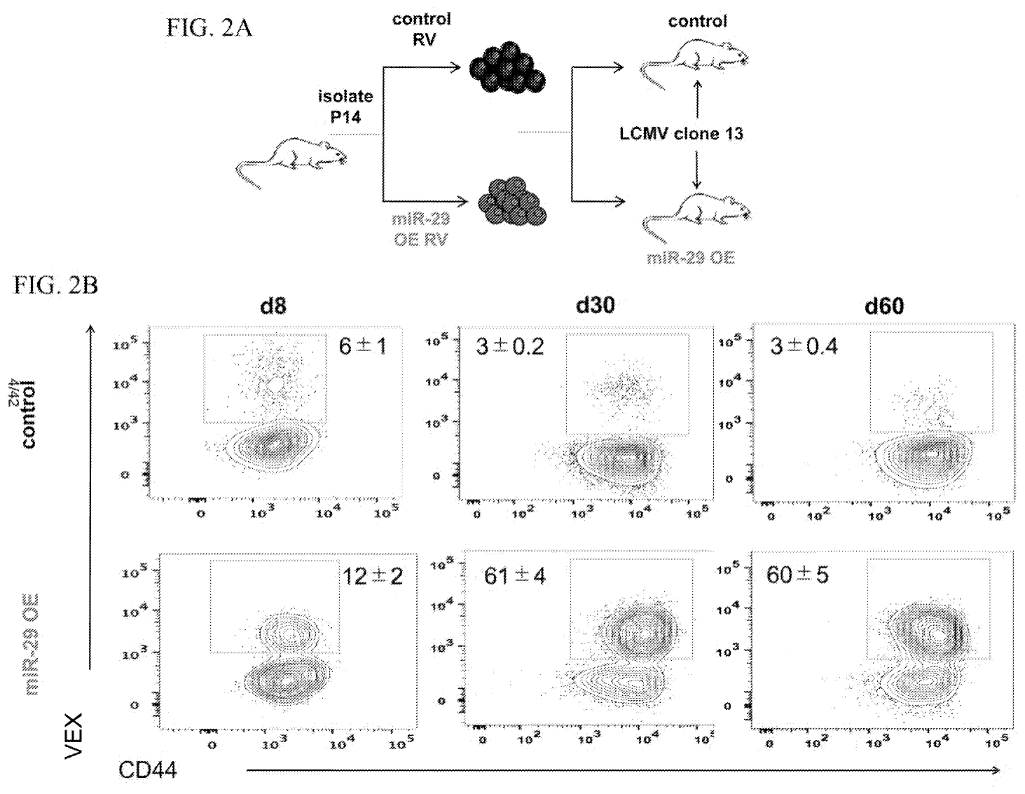

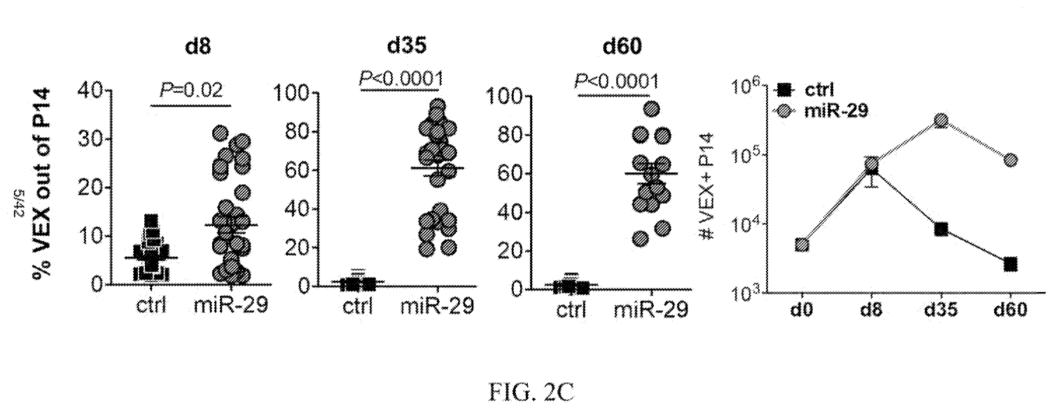

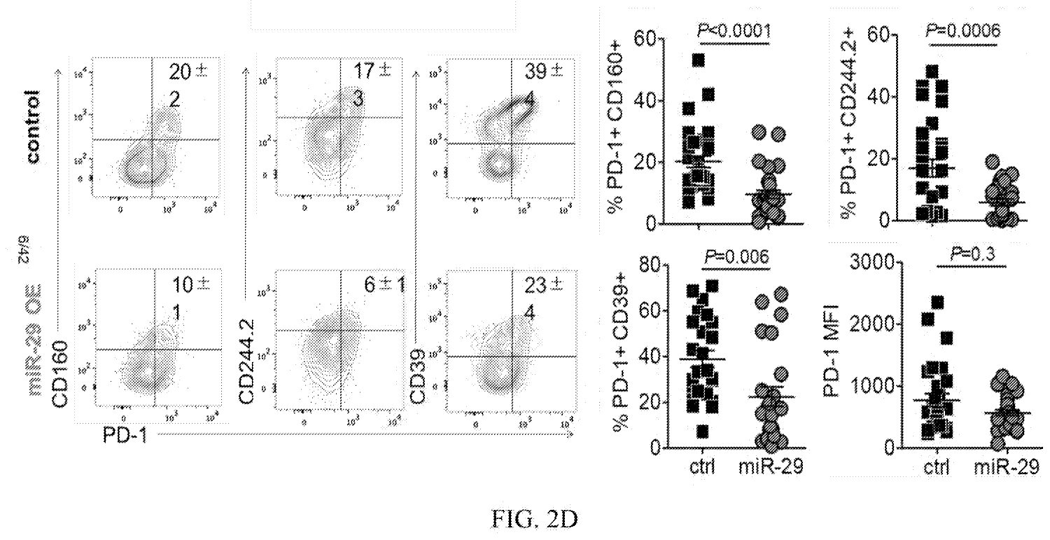

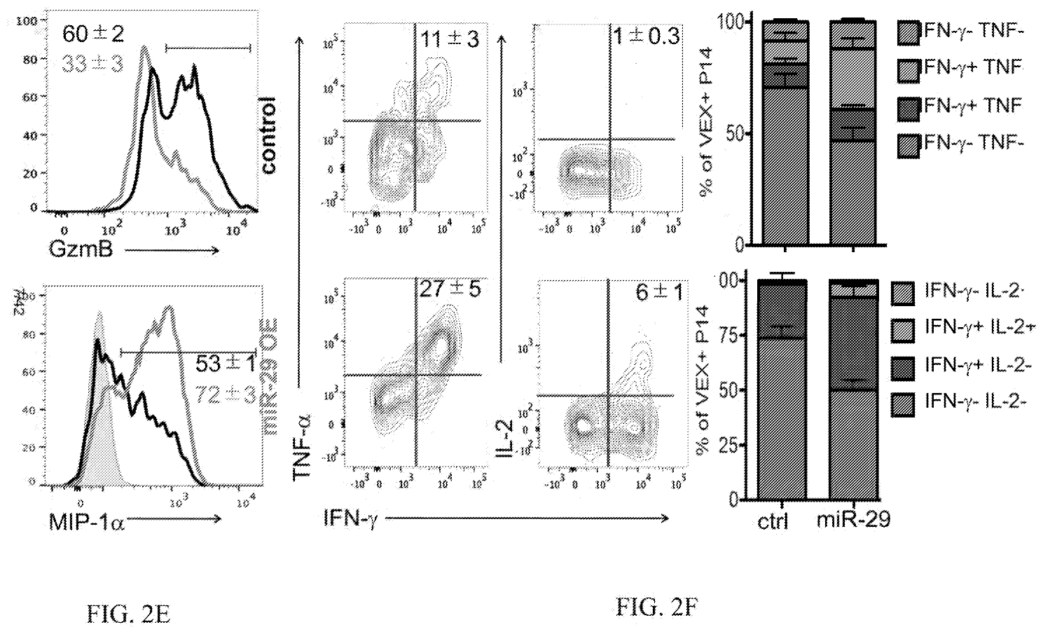

T_{EX} are characterized by a distinct transcriptional and epigenetic profile. Without wishing to to bound by theory, it was then hypothesized that miR expression patterns contribute to the T_{EX}-associated mRNA expression profiles, specifically due to the absence in T_{EX} of miRs that are upregulated in T_{MEM}. MIR-29a was a prime candidate as it was the only T_{MEM}-Specific miR identified in these analyses (FIG. 1A) and this miR was downregulated in CD8 T cells during chronic infection (FIG. 1B). Without wishing to be bound by theory, it was hypothesized that enforced expression of miR-29a might improve CD8 T cell responses during chronic infection by enforcing T_{MEM}-like differentiation. To test this, retroviral (RV) transduction was used to enforce expression of miR-29a in TCR transgenic CD8 T cells (P14) that recognize the LCMV D^{b}GP33-41 epitope. P14 cells were transduced with miR-29a RV or control RV and adoptively transferred into congenically distinct LCMV clone 13-infected recipient mice at dl p.i. (FIG. 2A). Despite equal transduction efficiency, transduction with miR-29a expressing RV increased the frequency and number of responding P14 cells (FIGS. 2B-2C) with an increasing advantage of the miR29a RV transduced P14 cells at 1 and 2 months p.i.

The numerical increase and enhanced persistence of miR-29a overexpressing (OE) P14 cells in chronic infection suggested that miR-29a may antagonize CD8 T cell exhaustion. Indeed, although miR-29a OE did not have a dramatic impact on expression of PD-1, expression of other inhibitory receptors was decreased resulting in substantial reduction in inhibitory receptor coexpression, a key feature of T_{EX} (FIG. 2D). This effect of miR29a OE on inhibitory receptor coexpression was not likely due to changes in viral load, because the number of P14 cells initially adoptively transfers was chose based on previous studies to not impact viral replication and viral load in serum and kidney at d30 p.i. was similar between the miR-29a OE and the control RV group. Moreover, inhibitory receptor expression by the non-transduced (VEX negative) P14 cells in each group was indistinguishable, consistent with a cell-intrinsic role for miR29a. T_{EX} maintain expression of granzyme B, but have reduced cytokine production upon stimulation, in contrast to T_{MEM}. MIR-29a OE P14 cells expressed less granzyme B (FIG. 2E) but had increased cytokine and chemokine production (FIG. 2E-F). Thus, miR29a OE promoted robust CD8 T cell expansion and persistence during chronic viral infection and antagonized key features of exhaustion.

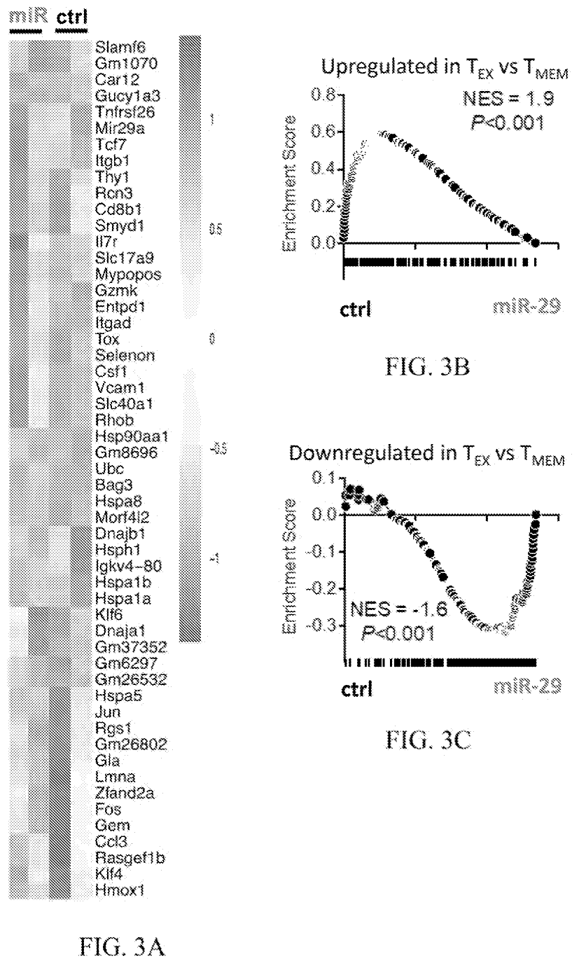

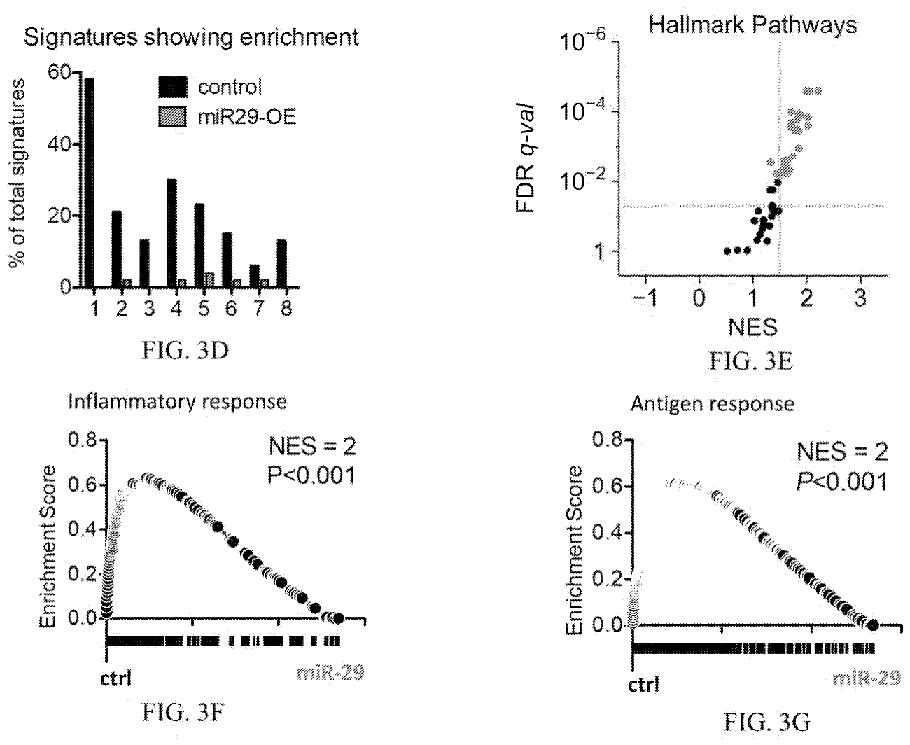

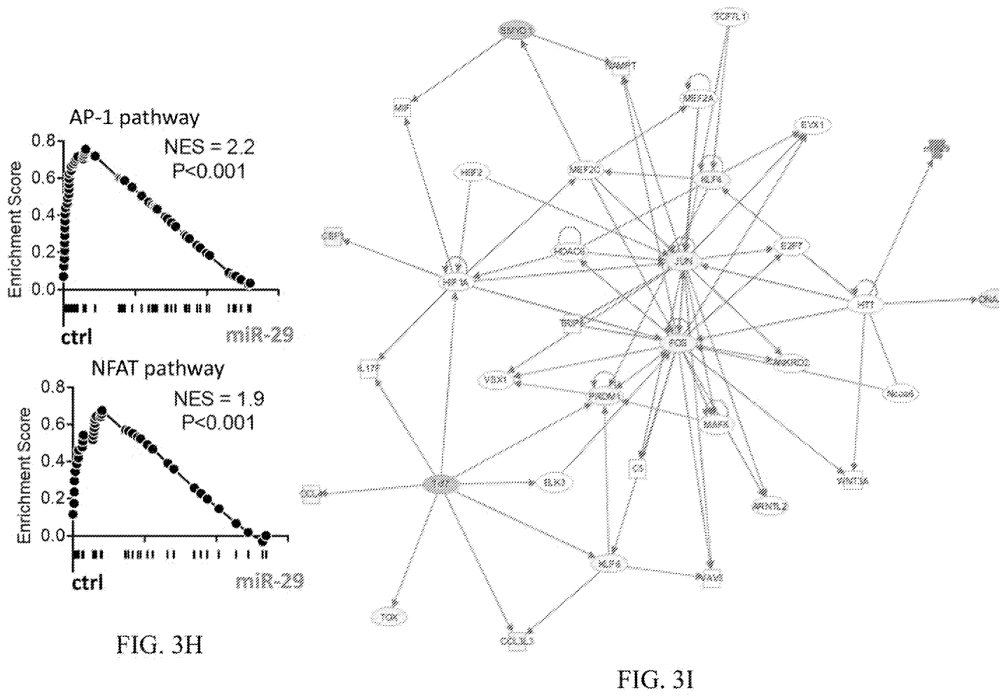

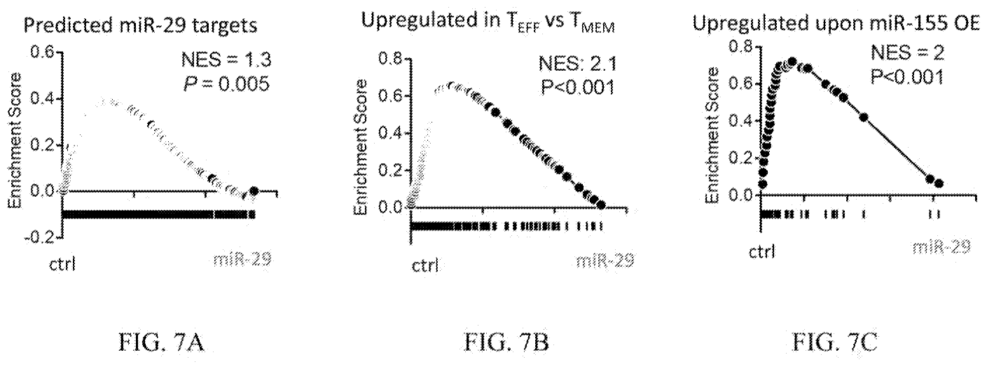

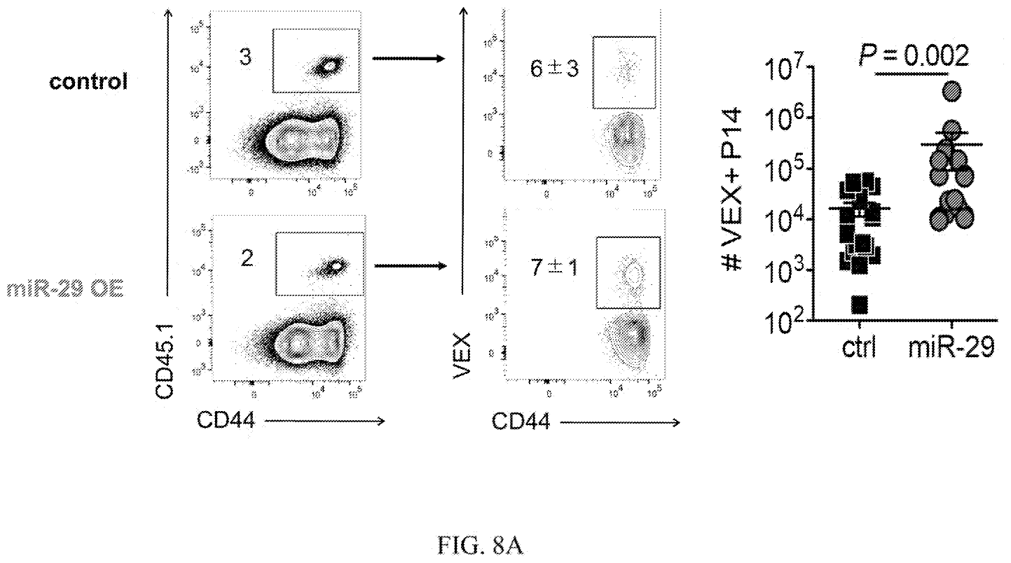

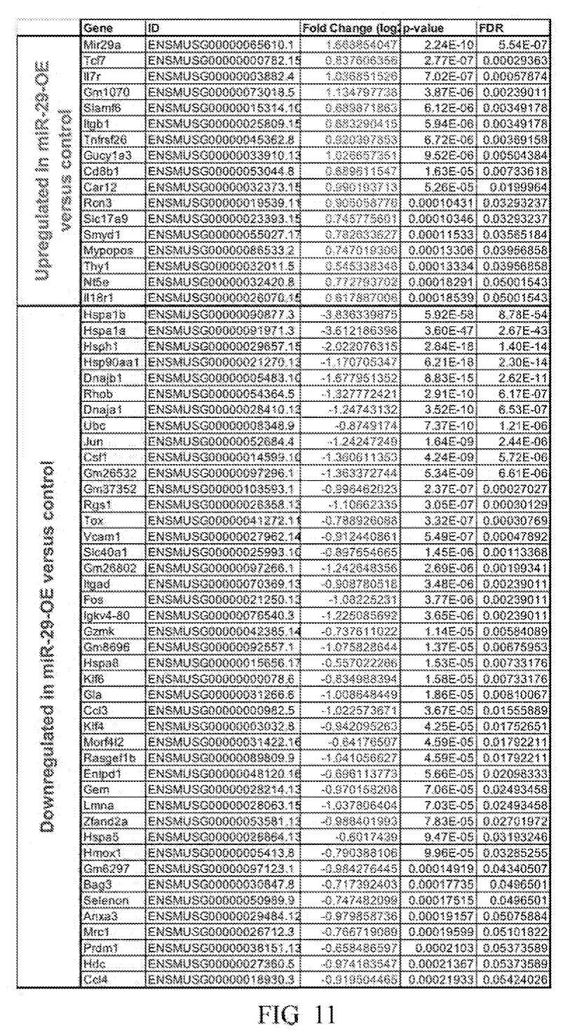

To begin to dissect the molecular mechanisms by which miR-29a antagonizes CD8 T cell exhaustion, the transcriptional program of miR-29a-OE and control RV transduced P14 cells was analyzed at d30 p.i. MiR-29a was upregulated 9-fold (FDR=5×10⁻⁷) confirming stable transduction and overexpression (FIG. 11). Sixty one transcripts were significantly changed (FDR<0.05) due to miR-29a OE (FIG. 3A). The majority of these transcripts (72%) were downregulated upon miR-29a-OE (FIG. 3A). Predicted miR-29a target genes were significantly enriched in the control RV group compared to miR-29a OE P14 cells (FIG. 7A). Downregulated genes in miR-29a OE P14 cells included transcription factors implicated in T_{EX} differentiation, such as Jun, Fos and Tox (FIG. 3A). Among the few transcripts that were upregulated upon miR-29a OE, were IL-7Ra and Tcf7, a key T_{MEM} promoting transcription factor. These transcriptional data are consistent with the cellular and functional data above and support the notion that miR-29a can antagonize exhaustion.

Since miR-29a was strongly associated with T_{MEM}, studies next asked whether miR-29a OE promoted a more global pattern of T_{MEM}-like differentiation during chronic infection. Indeed, Gene Set Enrichment Analysis (GSEA) revealed substantially reduced enrichment of T_{EX} associated genes in the miR-29a OE P14 cells (FIG. 3B). Similarly, genes that are downregulated in T_{EX} compared to T_{MEM} were strongly enriched in miR-29a OE P14 cells compared to control RV transduced P14 cells (FIG. 3C). Further, genes upregulated in T_{EFF} versus T_{MEM} were enriched in control versus miR-29a OE P14 cells, suggesting that miR-29a fostered differentiation of P14 cells towards T_{MEM} rather than T_{EFF} (FIG. 7B). One of the few miRs implicated in T_{EX} is miR-155. However, unlike miR-29a, miR-155 promotes durability, but not reversal of exhaustion. Therefore, it was asked whether miR-29a antagonized exhaustion by antagonizing the effects of miR-155. Indeed, the gene signature associated with miR-155 OE was enriched in control versus miR-29a OE P14 cells (FIG. 7C), suggesting that miR-29a antagonizes the effect of miR-155. Thus, enforced miR-29a expression in virus-specific CD8 T cells antagonizes a transcriptional profile associated with T_{EX} and fosters transcriptional features associated with T_{MEM}.

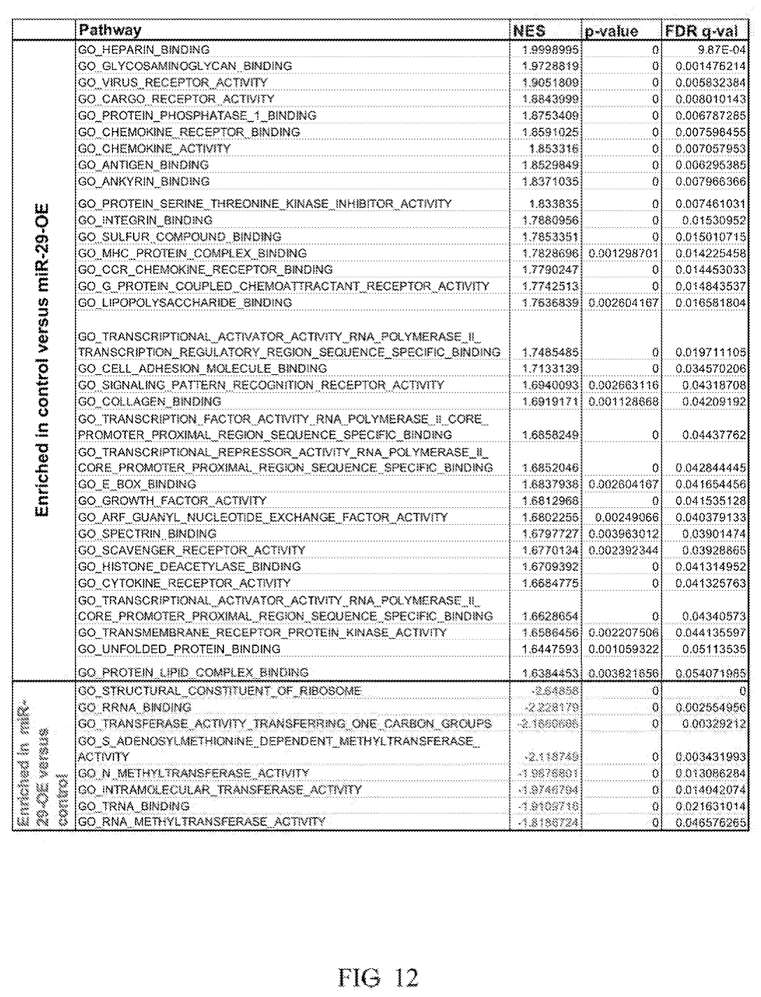

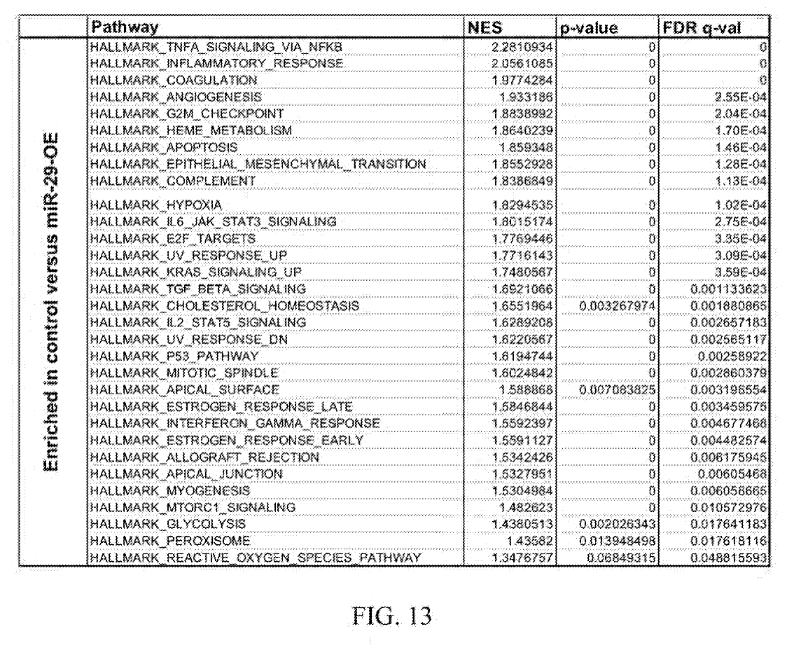

To further interrogate the underlying mechanisms by which miR-29a fosters T_{MEM}-like transcriptional, phenotypic and functional features during chronic viral infection, studies then examined the biological pathways and transcription circuits regulated by miR-29a. Only a small number of Hallmark, KEGG, BIOCARTA, or GO term biological pathways were enriched in miR-29a OE P14 cells at d30 p.i. (FIG. 3D-E), consistent with global mRNA downregulation as the major transcriptional effect of miR-29a OE (FIG. 3A). Among the few biological pathways induced by miR-29a were several related to ribosome biogenesis and protein translation (FIG. 12). Regulation of the translational machinery is critical for CD8 T cell differentiation and downregulation of genes encoding ribosomal subunits is a prominent feature of T_{EX} that may be associated with poor bioenergetics. A reversal of this feature of T_{EX} may contribute to better expression of effector molecules by miR-29a OE P14 cells. Several cytokine signaling and inflammatory pathways were also downregulated upon miR-29a OE in P14 cells during chronic infection (FIG. 3F and FIG. 13), suggesting that miR-29a may attenuate the response to inflammatory cytokines and thus, abrogate the deleterious effect of chronic inflammation on T_{MEM} differentiation. Moreover, a transcriptional signature of antigen stimulation was enriched in control versus miR-29a OE P14 cells (FIG. 3G) suggesting a potential role for miR-29a in limiting overstimulation of antigen-specific CD8 T cells that drives exhaustion during chronic infection. Thus miR-29a may antagonize exhaustion and promote T_{MEM}-like differentiation by regulating responses to both antigen and inflammation.

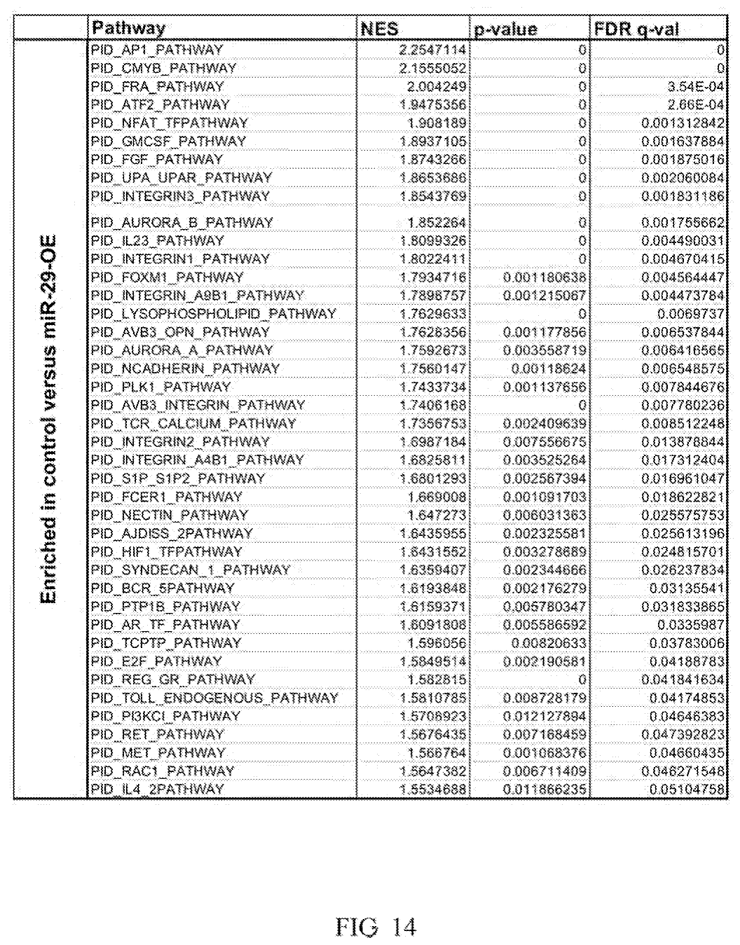

The impact of miR-29a OE on transcription factors, the downstream mediators of changes in inflammatory or TCR signaling pathways was next investigated. The Pathway Interaction Database (PID), a collection of cellular signaling pathways and intracellular molecular interactions was used. Although none of the 180 gene sets from the PID database enriched in miR-29a-OE P14 cells, 41 PID pathway gene sets enriched in control RV transduced P14 cells suggesting downregulation of these pathways by miR-29a OE. These pathways included key transcription factor pathways, such as AP-1, c-Myb and NFAT as well as the TCR_CALCIUM pathway (FIG. 3H, FIG. 14). Indeed, a transcriptional network involving Fos and Jun, as well as the exhaustion related transcription factors Prdm1 and Tox was significantly affected by miR-29a OE (FIG. 3I). This network also included differential expression of Klf4 and Tcf7, two transcription factors implicated in T_{MEM} differentiation. Collectively, these results suggest a novel role for miR-29a as a central regulator of key transcriptional networks in CD8 T cells, acting as a rheostat between central exhaustion pathways (Tox/AP-1) and memory-associated pathways (Tcf7).

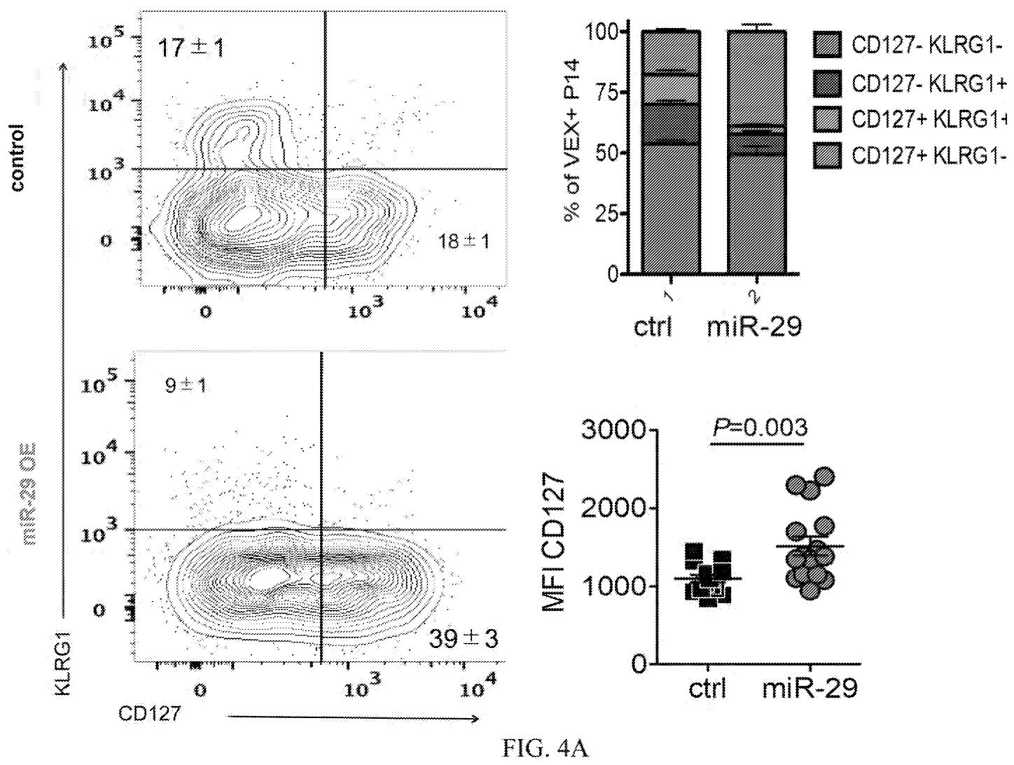

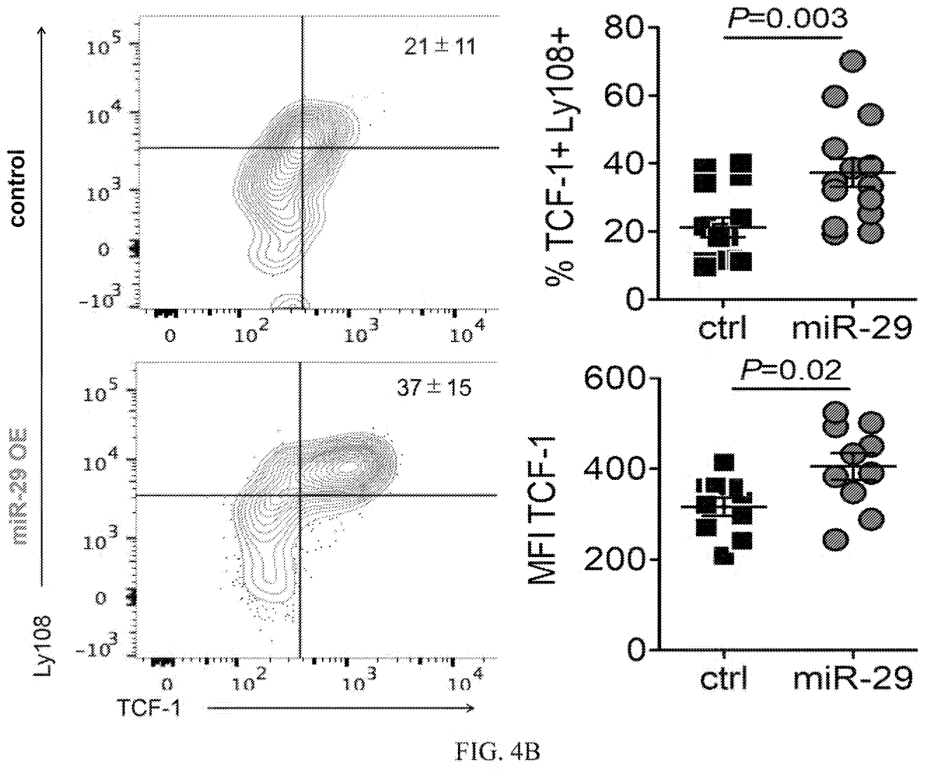

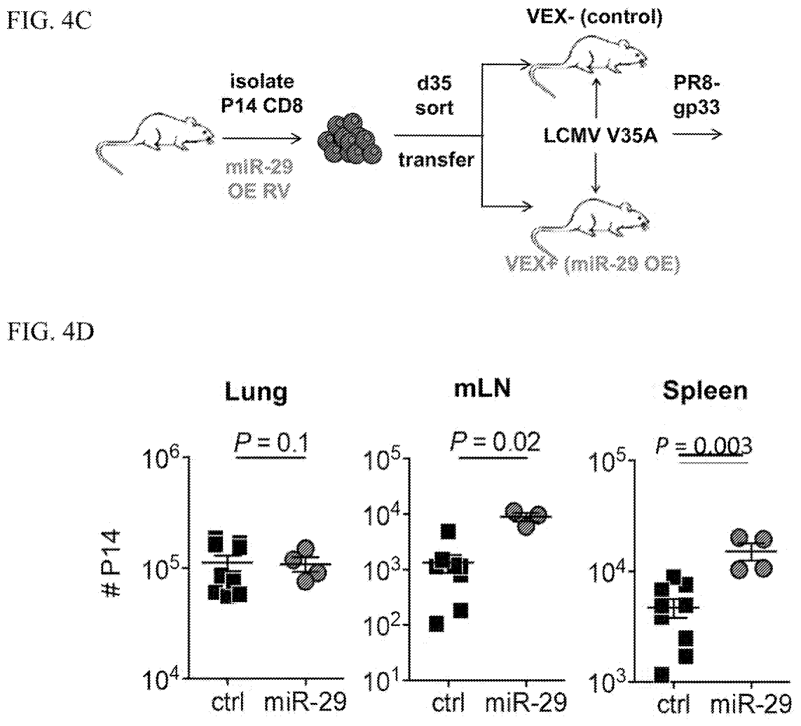

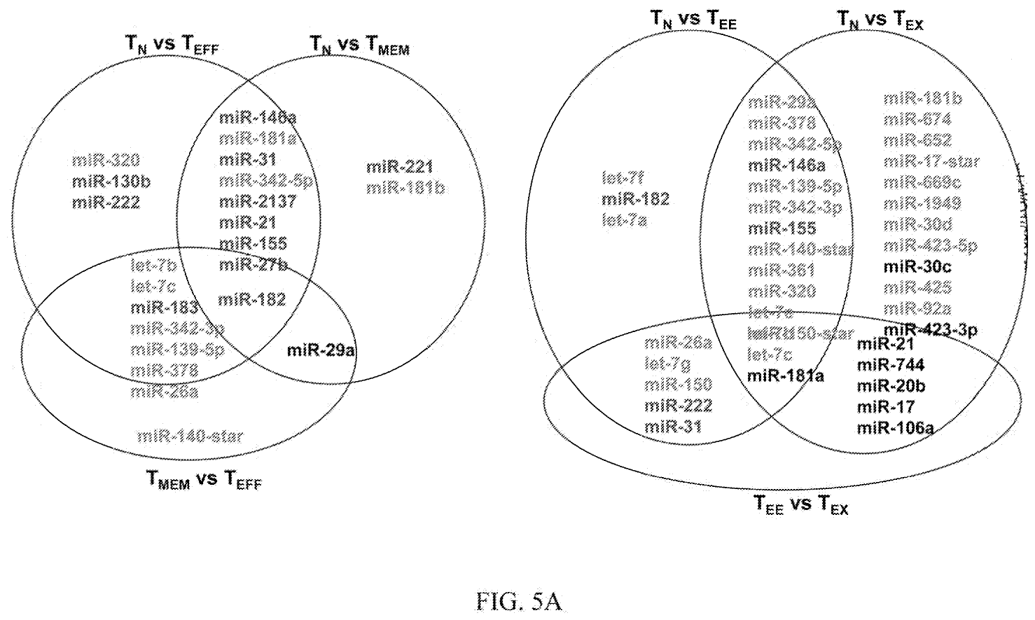

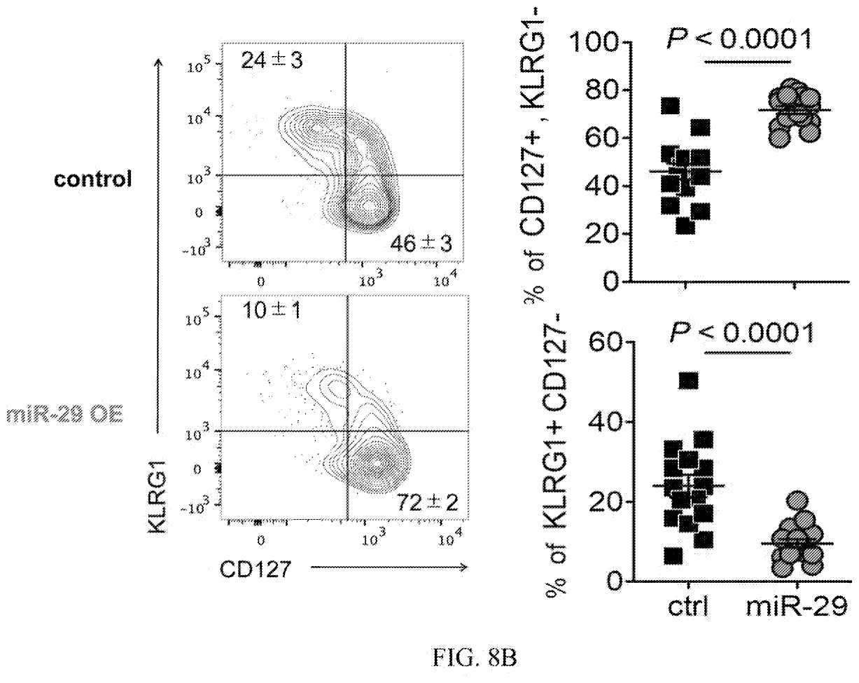

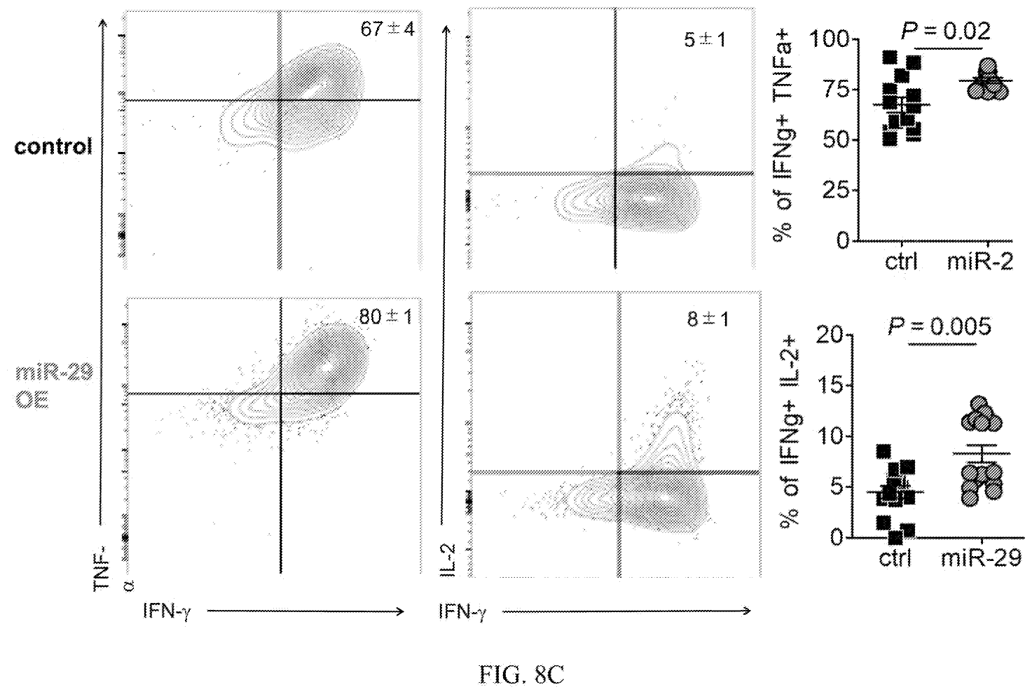

The data above provokes the hypothesis that enforced miR-29a expression may foster T_{MEM}-like differentiation and function in virus-specific CD8 T cells during chronic infection. Therefore, the impace of miR-29a OE on the development of other phenotypic and functional T_{MEM} properties during chronic viral infection was examined. Indeed, miR-29a OE enhanced expression of the memory-associated molecule IL-7Rα by P14 cells in chronic LCMV infection (FIG. 4A). Expression of the T_{EFF} marker KLRG1 by T_{EX} is typically low, but miR29a OE further reduced expression of this molecule consistent with a shift towards T_{MEM} or a memory precursor cell (IL-7Rα+ KLRG1−) differentiation state (FIG. 4A). This effect of miR-29a OE on P14 cell differentiation in chronic infection promoted a pattern of IL-7Rα and KLRG1 expression similar to what was observed during acutely resolved infection (FIG. 8). The transcription factor TCF-1 plays a key role in both long-term T_{MEM} following acutely resolved infections and also in T_{EX} progenitor cells during chronic infections and cancer (35-37). Enforced expression of miR-29a enhanced TCF-1 expression in P14 cells during chronic infection (FIG. 4B), consistent with a shift towards progenitor or T_{MEM}-like differentiation.

A canonical property that distinguishes T_{MEM} from T_{EFF} or T_{EX} is the ability of T_{MEM} to mount robust recall responses upon re-infection. It was therefore tested whether miR-29a OE in chronic infection improved recall responses. miR-29a OE and non-transduced P14 cells were purified from chronically infected mice at d30 p.i. Equal numbers of control or miR29a OE P14 cells were then adoptively transferred to new congenic recipient mice. To avoid the potential caveat of infecting these secondary recipients with LCMV clone 13 associated with the adoptively transferred P14 cells, recipient mice that had been previously infected with LCMV V35A, a variant of LCMV that lacks epitope gp33 were used. These secondary recipients were then challenged intranasally 2 days later with influenza PR8 expressing the GP33-41 epitope (PR8-GP33) (FIG. 4C). The P14 cells with enforced expression of miR-29 mounted a more robust recall response upon reinfection compared to the control P14 populations (FIG. 4D). This effect was detected mainly in the mLN and spleen but not lungs as expected for T_{MEM}-like responses in lymphoid tissues (FIG. 4D). Thus, these data are consistent with the transcriptional and phenotypic changes driven by miR-29a OE in chronic infection and suggest that miR-29a can foster improvements that allow improved recall responses to be preserved despite the persistent antigen stimulation of chronic infection. Together, without wishing to be bound by theory, these results suggest miR-29a as a potential therapeutic target for enhancing T_{EX} function and diverting T_{EX} differentiation towards more T_{MEM}-like differentiation in cancer and chronic infections.

### Example 2: Discussion

In the studies of the present disclosure, miR-29a was identified as a novel molecule that attenuates exhaustion and enhances persistence and function of CD8 T cells during chronic viral infection. Mechanistically, a role for miR-29a was identified as a rheostat between exhaustion-related (AP-1, NFAT, Tox) and memory-related (TCF-1) transcriptional pathways, that are implicated in T_{MEM} versus T_{EX} differentiation. Moreover, these data suggest that miR-29a functions by attenuating TCR and/or inflammatory signaling pathways that feed into these key transcriptional circuits, consistent with the known importance of overstimulation driving T cell exhaustion. Together these studies suggest that enhanced expression of miR-29a may be a novel strategy to foster more functional, durable T_{MEM}-like differentiation in the context of persistent antigen stimulation, such as chronic infections and cancer. A major gap in current understanding of T cell exhaustion has been defining the roles of non-coding RNAs including miRs. MiRs can simultaneously target several mRNAs and, therefore, modulating expression of a single miR could have broader biological impact than modulating expression of individual mRNAs. The global miR profiling here revealed novel patterns of miR expression in T_{EFF}, T_{MEM} and T_{EX} in vivo, especially miR-29a.

T_{EX} reinvigoration by checkpoint blockade has had remarkable clinical success. Despite these successes many patients do not benefit from durable clinical responses and recent data suggest that the immunological response to checkpoint blockade may be transient. In other words, PD-1 pathway blockade may not induce long-term T_{EX} reinvigoration or T_{MEM}-like differentiation. Optimal immunotherapies aimed at reversing or preventing exhaustion may, therefore, need to address issues related to acquisition of T_{MEM}-like properties to optimally enhance durability, persistence and recall capacity. The effects of miR-29a on quantitively and qualitatively improving T_{EX} responses and inducing phenotypic, functional and transcriptional changes are consistent with T_{MEM}-like differentiation and suggest changes in central pathways involved in the dichotomous T_{MEM} versus T_{EX} differentiation states. Indeed, miR-29a OE resulted in lower expression of Tox, the epigenetic inducer of T_{EX} differentiation. MiR29a OE also upregulated expression of Tcf7, the key transcription factor that governs T_{EX} progenitor cells during chronic infection and is necessary for responses to anti-PD1 therapy. TCF-1 (encoded by Tcf7) is also a major regulator of long-term, quiescent central memory CD8 T cells potentially connecting this effect of miR29a OE to the improved memory-like properties observed. Thus, the studies of the present disclosure demonstrate miR-29a as a key player of CD8 T cell differentiation by regulating transcriptional pathways central to T_{MEM} versus T_{EX} differentiation.

These data on miR-29a promoting persistence of CD8 T cells during chronic infection complement current understanding of how another miR, miR-155, functions to promote long-term persistence of T_{EX}. The underlying mechanisms of how these two miRs alter T_{EX} differentiation are different, yet complementary. MiR-155 enhances long-term persistence of exhausted CD8 T cells by increasing surface inhibitory receptor expression and, therefore rendering the cells less susceptible to the deleterious effects of persistent TCR and inflammatory signals. On the contrary, miR-29a enhances long-term persistence by directing the cells to a T_{MEM}-like phenotype and altering expression of transcription factor pathways, downstream of TCR, such as Jun, Fos, NF-kB, Tox, and NFAT. In fact, miR-29a antagonized the effect of miR-155, suggesting opposing mechanisms by which two individual miRs regulate T_{EX} differentiation.

Whereas miR-155 inhibits the responsiveness to external stimuli by increasing inhibitory receptor expression, miR-29a affects downstream molecular pathways. In both cases, TCR and inflammatory signaling are inhibited, in line with the known role of TCR and inflammatory signaling in driving exhaustion. However, whereas miR-155 allows exhausted CD8 T cells to withstand the stress of overstimulation and persist despite this chronic activation, miR-29a prevents CD8 T cells from entering into the state of full exhaustion by limiting pathways driving the primary overstimulation signal. The mechanistically different roles of miR-29a and miR-155 in regulating T_{EX} further poses the question of a potential synergistic effect in CD8 T cell persistence and differentiation. Further, this notion of two miRs affecting the same problem of overstimulation that leads to exhaustion by employing two distinct mechanisms suggests new opportunities to prevent and/or reverse exhaustion by controlling antigen and inflammatory signaling. It will be interesting in the future to dissect how miR-29a impacts the induction and/or stability of the epigenetic landscape of T_{EX} and determining whether de novo expression of miR29a once exhaustion has been established impacts reversal of exhaustion or reprogramming of T_{EX}.

In conclusion, the studies of the present disclosure have identified a major role for miR-29a in regulating T_{EX} differentiation, promoting long-term persistence and fostering a T_{MEM}-like differentiation state in CD8 T cells responding to chronic viral infection that would otherwise become exhausted. Thus, without wishing to be bound by theory, the present studies suggest that miR-29a represents a novel immunotherapeutic target to promote long-term, functional CD8 T cell responses in chronic infections and cancers, including for cellular therapies.

### Example 3: Utilizing miR-29a to Augment CAR T Cell Function

The present disclosure identifies miR-29a as a key regulator of the development of T cell exhaustion. These studies further demonstrate the use of miR-29a to maintain CD8 T cell function in immune microenvironments that tend to favor the development of T cell exhaustion, particularly through chronic antigen stimulation. One example of such a microenvironment is in tumors. Recent advances in the immunotherapy of both solid and liquid tumors have identified the exhaustion of effector cells as a major limitation to their clinical efficiacy. As a result, a series of studies was undertaken herein to incorporate the forced expression of miR-29a into CD8 T cells expressing chimeric antigen receptors specific for CD19, a tumor antigen associated with B cell lymphomas including acute lymphoblastic leukima (ALL) and chronic lymphocytic leukemia (CLL). CD19-specific CARs were the first approved CAR-based therapies for these diseases, and despite considerable clinical success, treatment often fails due to exhaustion of CAR-expressing T cells.

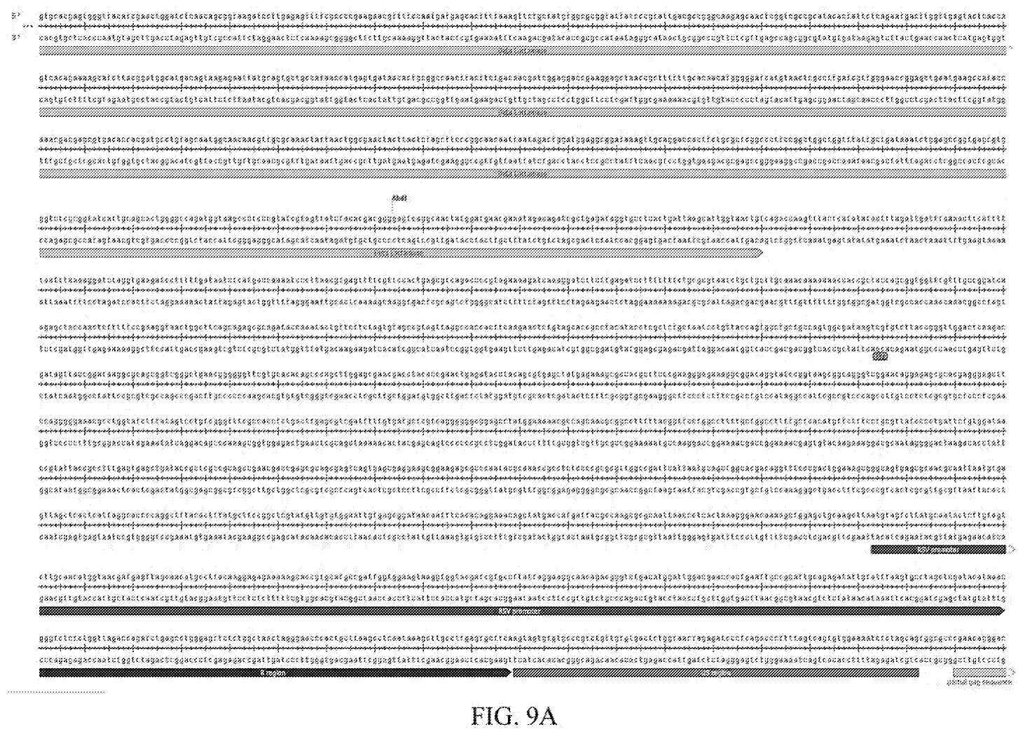

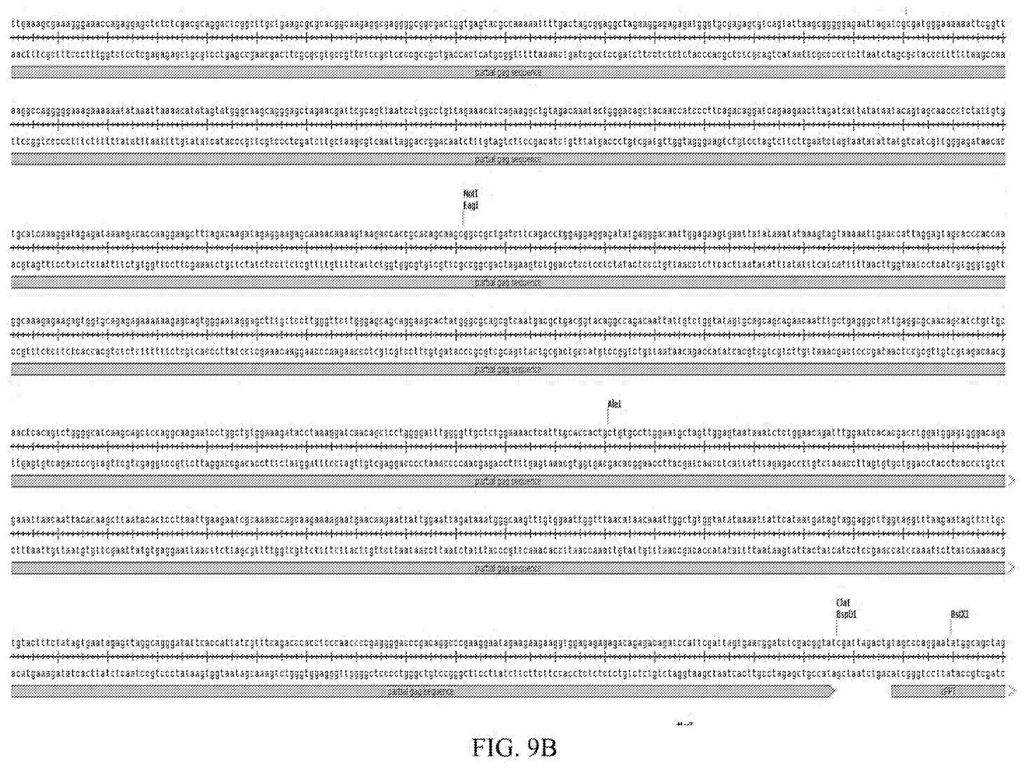

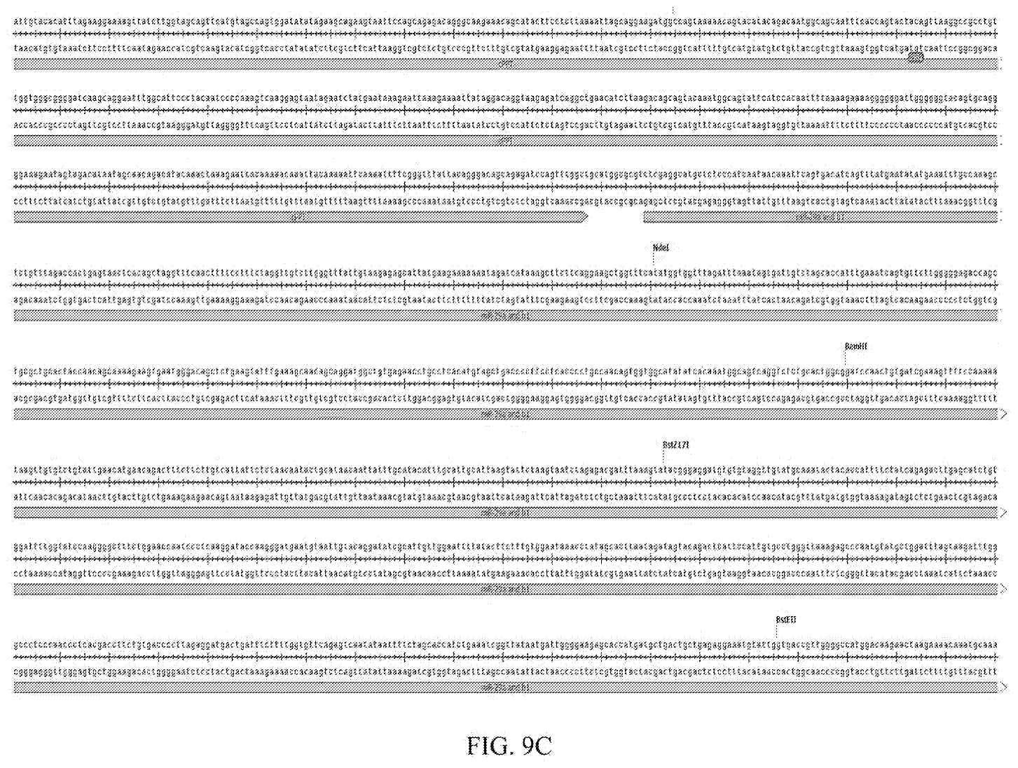

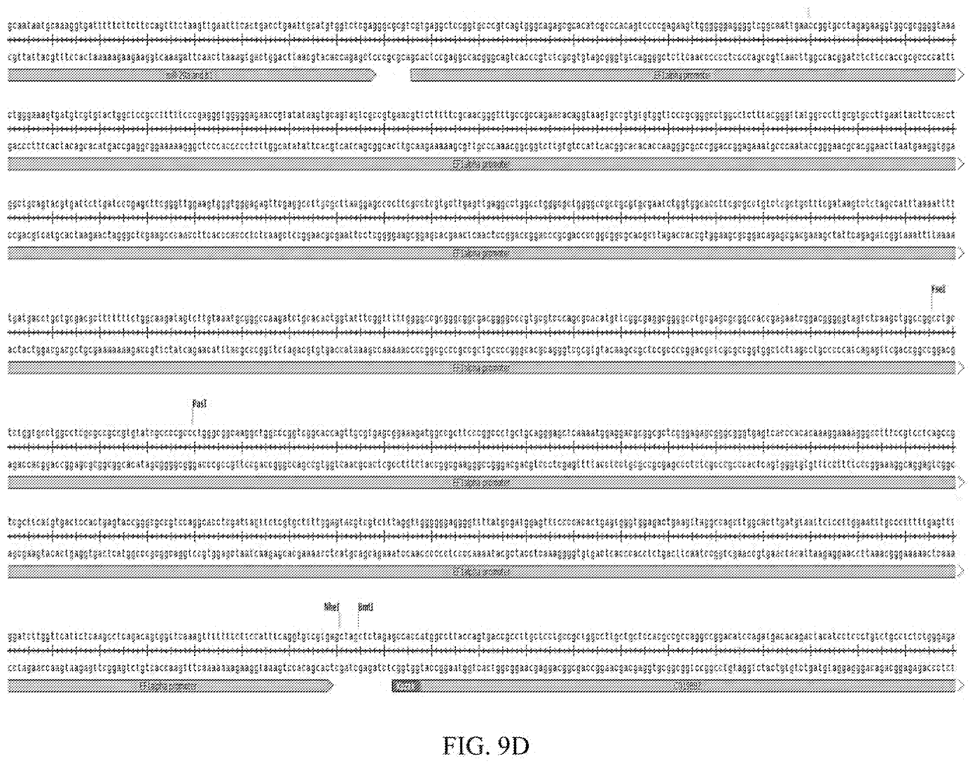

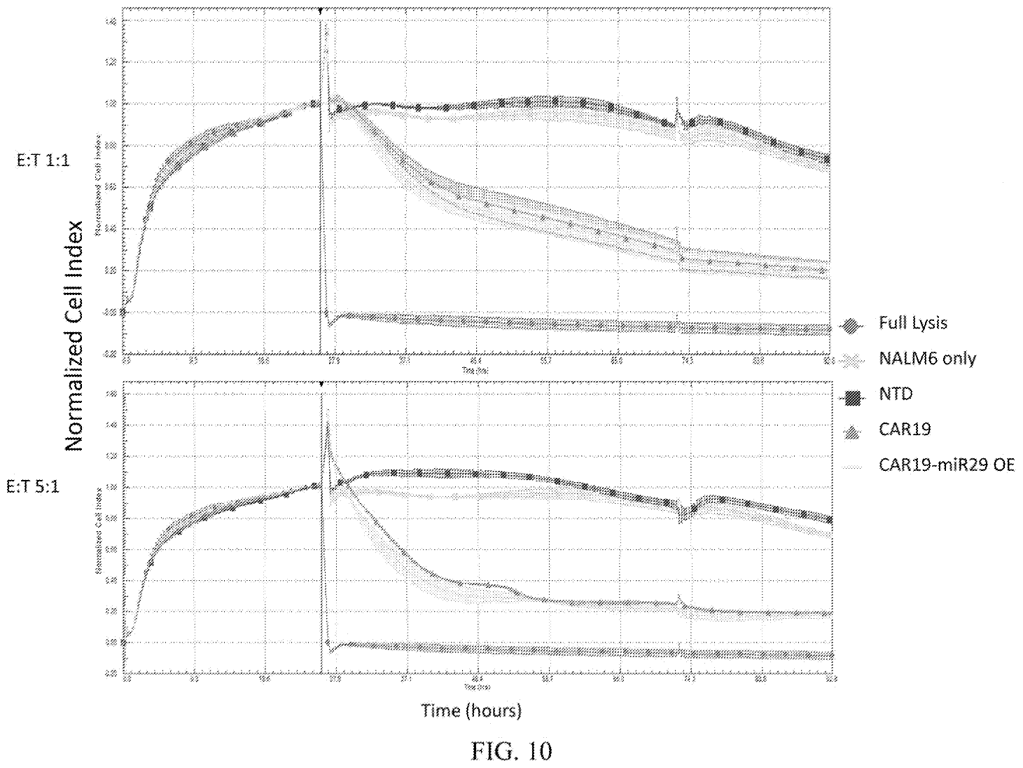

A CD19-specific CAR construct was modified to contain a nucleic acid sequence encoding miR-29a such that modified T cells would express both the CAR and miR-29A microRNAs (see the map in FIGS. 9A-9D). Modified cells were then used in in vitro cytotoxicity assays against CD19-expressing cell lines (FIG. 10), which demonstrated that the miR-29a expressing CAR constructs maintained their antigen specificity similar to T cells expressing unmodified CD19-specific CAR constructs. Notably, the expression of miR-29a in these cells did not significantly increase their cytoxic ability, though these assays were not conducted over a long enough time period for the onset of exhaustion to appreciably affect the results. Without wishing to be bound by theory, these data further suggest that the benefit of miR-29a expression lies not with increase cytotoxic function, but prolonged persistence and function in exhaustion-inducing environments.

### Example 4: miR-29a Attenuates Exhaustion, Promotes Memory-Like CD8 T Cell Differentiation, and Synergizes with PD-L1 Blockade to Promote Long-Term Persisting Memory-Like CD8 T Cell Responses

The fact that responses to immunotherapy are not sustained remains a problem. The profilerative burst provided by anti-PD1/PDL-1 therapy is only transient. Patients need to receive immunotherapy continuously, and therefore are exposed to many side-effects. If immunotherapy is stopped, the clinical benefit is usually lost. This is because current immunotherapeutics cannot induce long-term persisting memory T cell responses and cannot reverse the epigenetic profile of exhaustion.

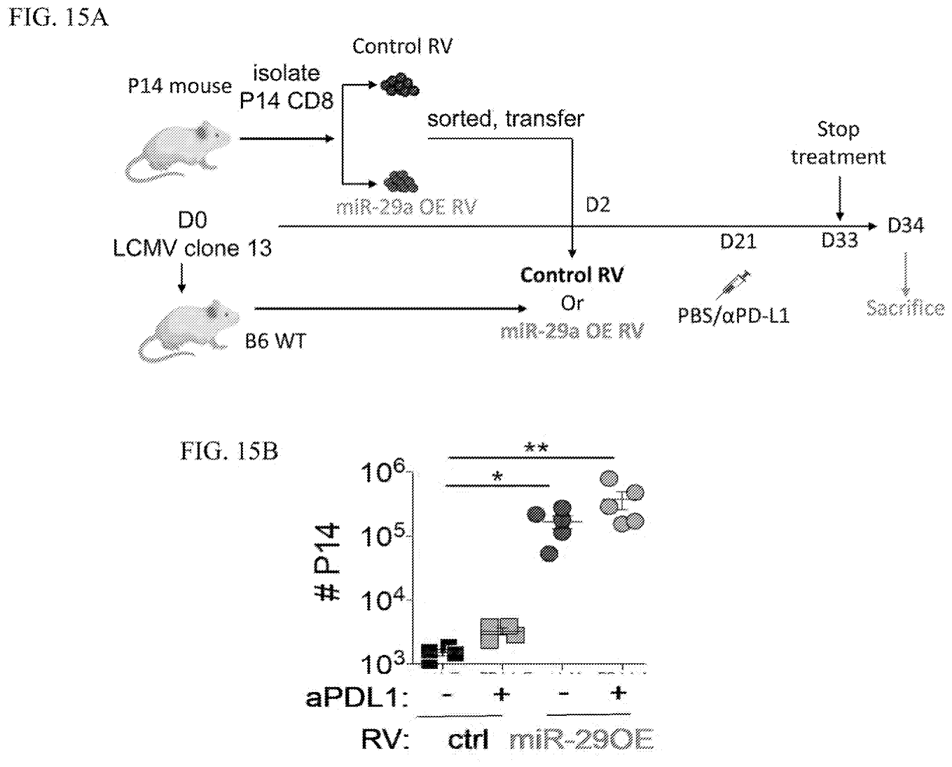

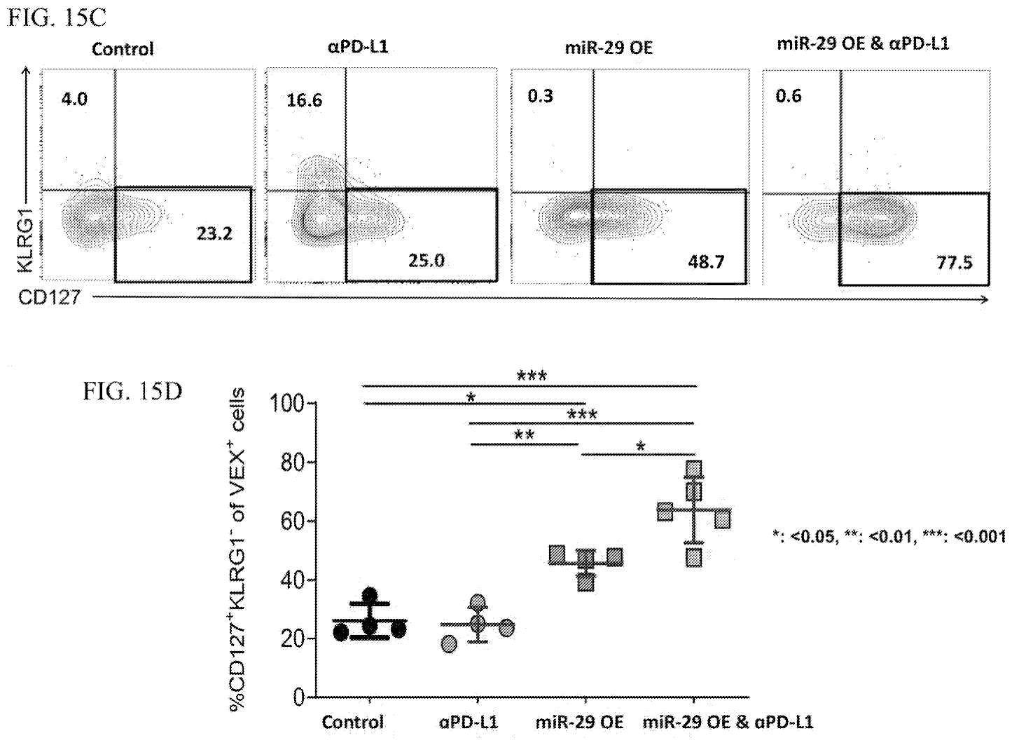

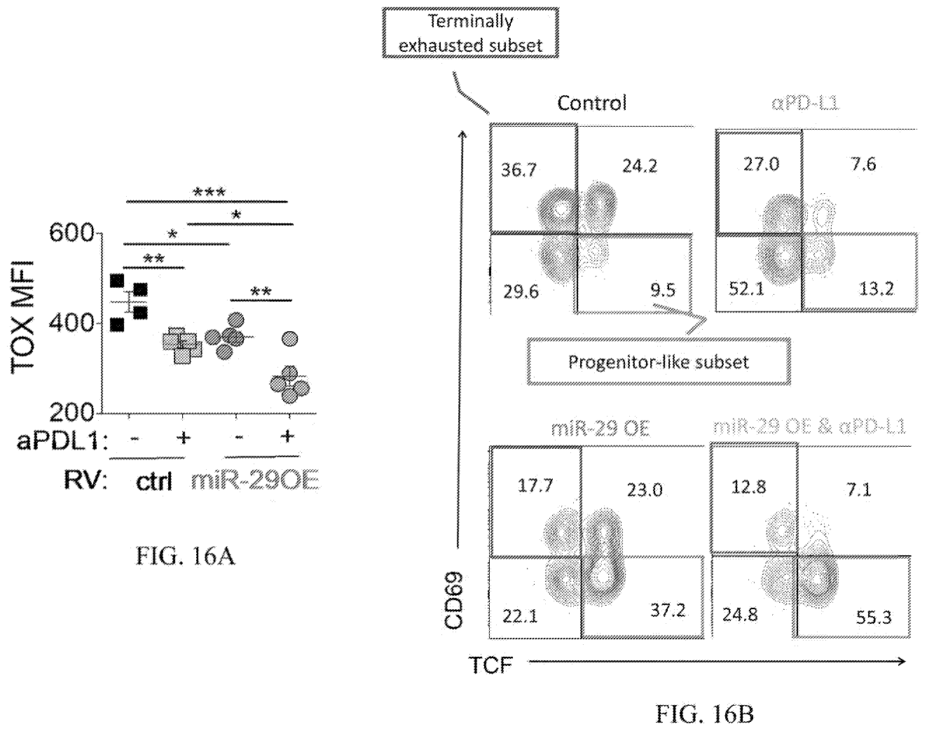

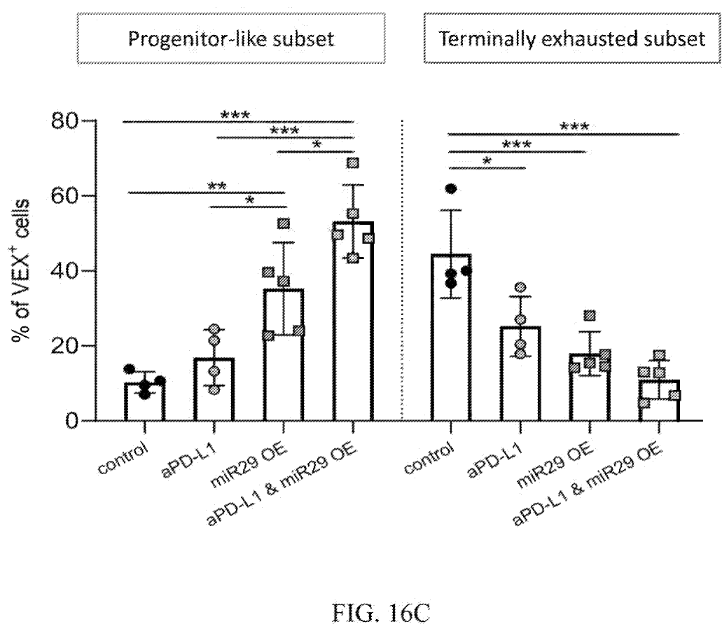

Checkpoint inhibitor blockades, such as anti-PDL-1, provide significant, though transient, clinical benefit. The progenitor T_{EX} subset expressing TCF-1 is the main subset responding to PD-1 blockade, and is thus responsible for T_{EX} reinvigoration upon immunotherapy. Since miR-29a promotes TCF-1 expression, it was hypothesized that miR-29a enhances the progenitor T_{EX} subset and, thereby, pre-conditions T_{EX} for optimizing responses to PD-1 blockade. To test this, equal numbers (3×10⁴) of miR-29a OE and control transduced cells were sorted and co-transferred to LCMV cl-13 infected recipients at dl p.i. (FIG. 15A). At d21 p.i. until d35 p.i. recipients were treated with 200 μg anti-PDL1 every 2 days. VEX+P14 cells were analyzed in spleens at dl after the last anti-PD-L1 treatment (FIG. 15B, FIG. 16C).

MiR-29a OE reduced expression of the master regulator of exhaustion, Tox, consistent with the role of miR-29a in attenuating exhaustion (FIG. 16A). Anti-PDL-1 treatment in the presence of miR-29a OE increased the number of P14 cells and CD127 expression and further decreased Tox expression compared to isotype treated mice (FIGS. 15B-15D, FIG. 16A). Importantly, miR-29a in combination with anti-PDL-1 further enhanced the generation of the progenitor TCF-1+ subset, suggesting that these responses may be long-lasting (FIG. 16B). These data demonstrate that miR-29a enhances responses to PD-1 blockade. Further, since T_{EX} differentiation depends on Tox, these data suggest that miR-29a in combination with anti-PDL-1 treatment fundamentally alters T_{EX} differentiation.

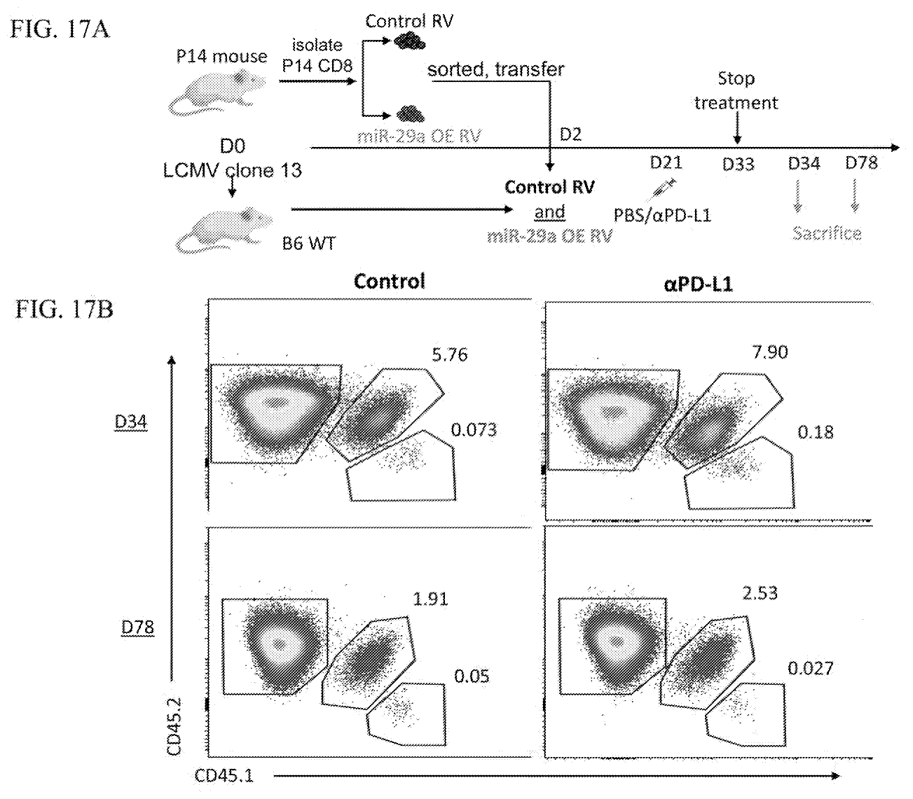

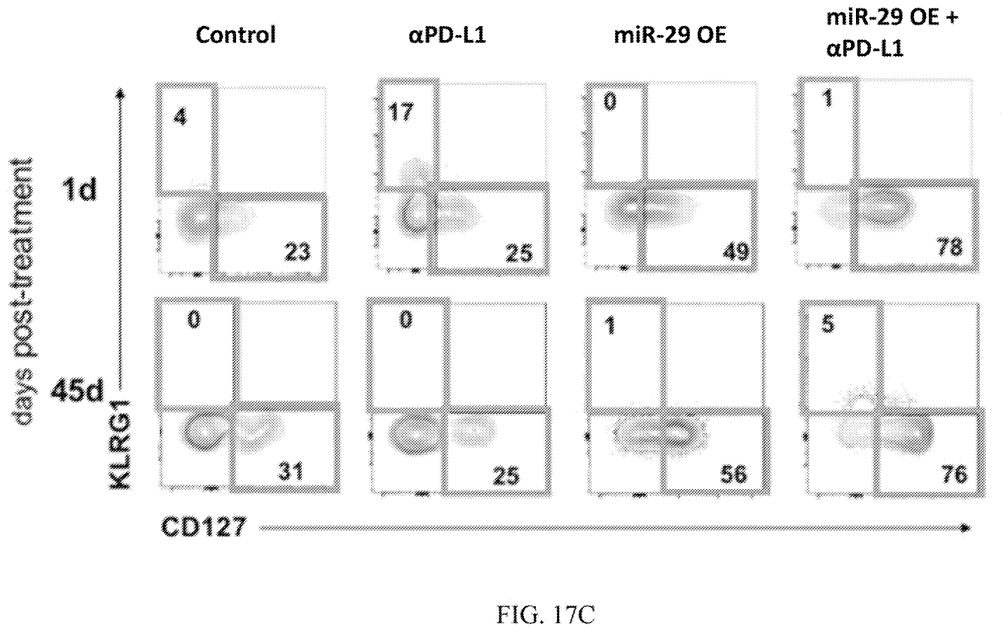

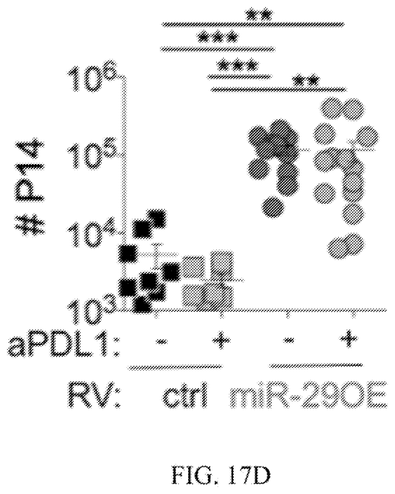

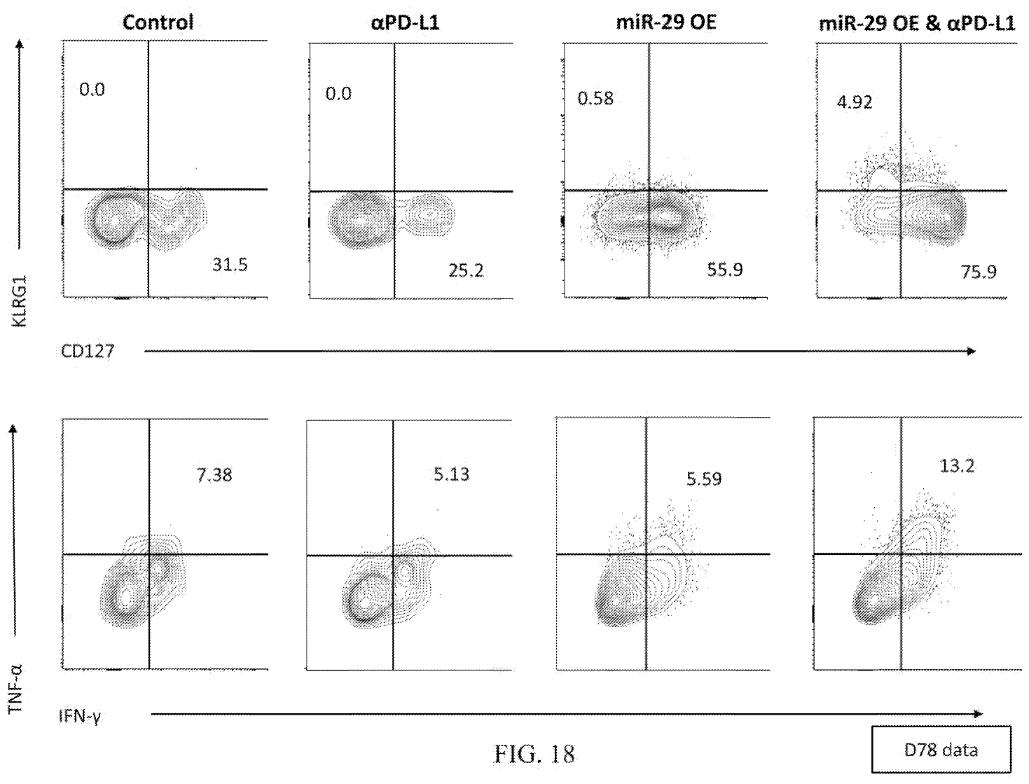

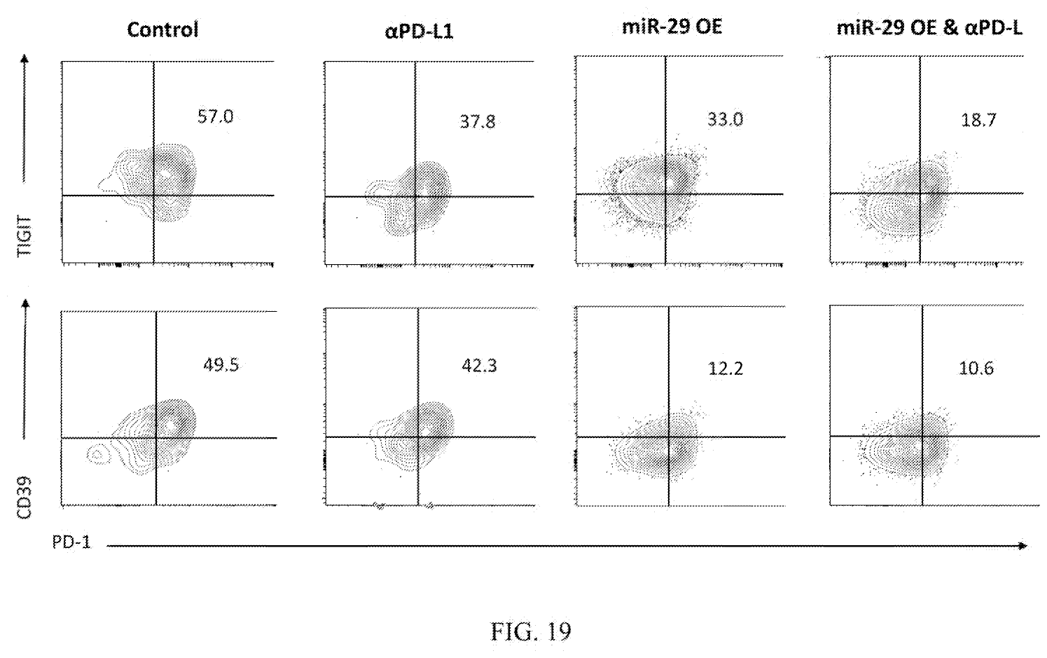

Checkpoint inhibitors provide only a transient benefit, and T_{EX} reinvigoration is not sustained. Since miR-29a-OE induced T_{MEM}-like differentiation, it was hypothesized that miR-29a OE can induce a sustained T_{EX} reinvigoration. 3×10⁴ miR-29a OE and control transduced P14 cells were co-transfered to LCMV cl-13 infected mice at dl p.i. At d21-d35 p.i. mice were treated with 200 mg anti-PD-L1 every 2 days i.p. VEX+P14 cells were analyzed in spleens at dl and d45 after the last anti-PD-L1 treatment (FIGS. 17A-17D). Indeed, increased numbers of miR-29a OE P14 cells were sustained even at 45d after cessation of anti-PD-L1 treatments (FIG. 17D). Importantly, while miR-29a-OE induced T_{MEM} (CD127+ KLRG1−), the combination of miR-29a-OE and anti-PD-L1 induced T_{EFF} differentiation (KLRG1+CD127−) without compromising the effect of T_{MEM} differentiation induced by miR-29a OE (FIG. 17C). In fact, miR-29a and anti-PD-L1 therapy had a syntergistic effect in sustaining memory-like T cells and enhancing effector functions (FIG. 18). miR-29a and anti-PD-L1 therapy retained exhaustion-associated receptors at low levels even after the cessation of PD-L1 blockade. These data show that the combination of miR-29a OE and anti-PD-L1 can induce robust and sustained T_{EX} reinvigoration by promoting both T_{MEM} differentiation and the generation of T_{EFF} long-term, even after cessation of treatment.

### Example 5: Antigen-Specific or Non-Antigen Specific miR-29a Overexpressing CD8 T Cells Provide Control Tumor Growth in Three Separate Preclinical Tumor Models

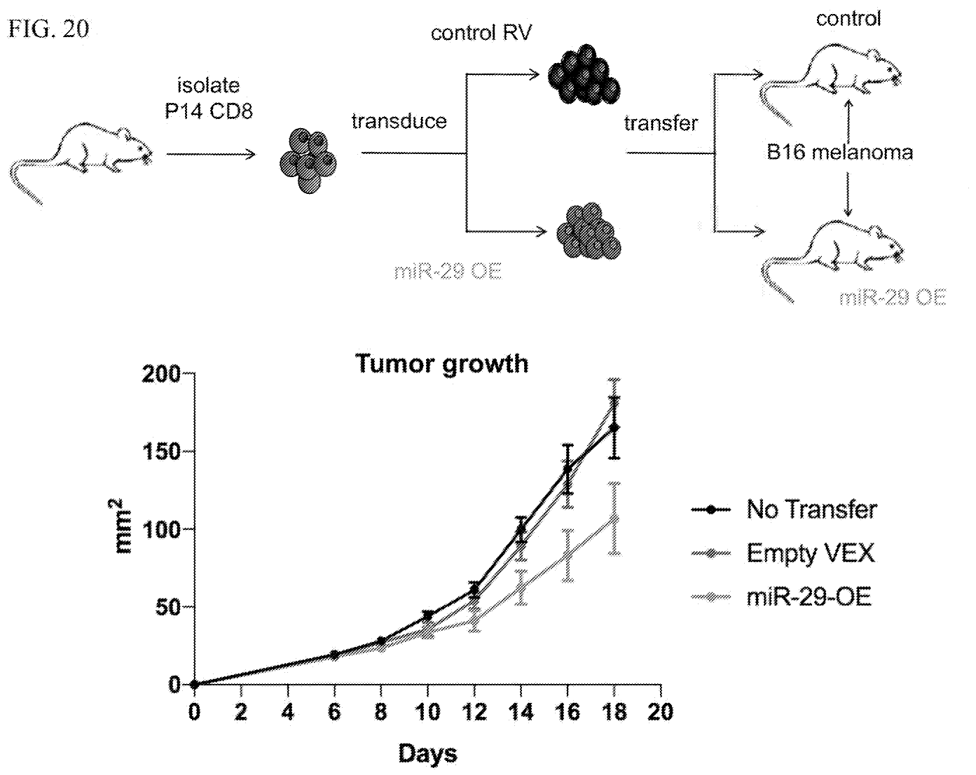

5×10⁵ miR-29a-OE VEX+ or control VEX+P14 cells were adoptively transferred i.v. into mice s.c. inoculated with 5×10⁵ B16-gp33 melanoma tumor cells at d5 (n=8). CD8+ T cells that overexpress miR-29 limited tumor growth (FIG. 20).

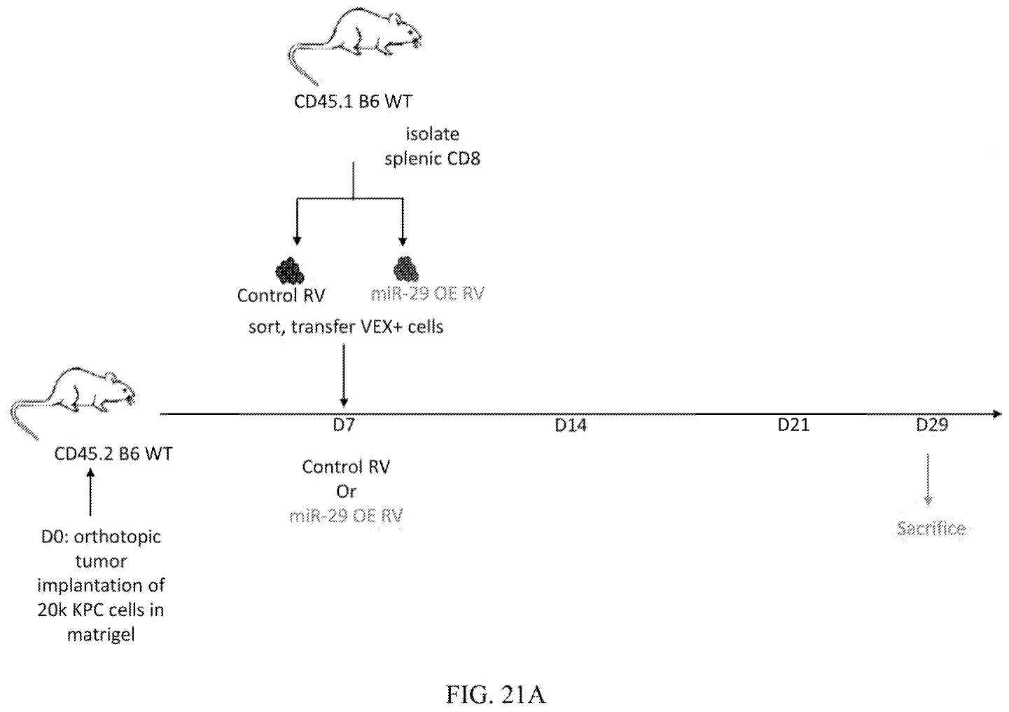

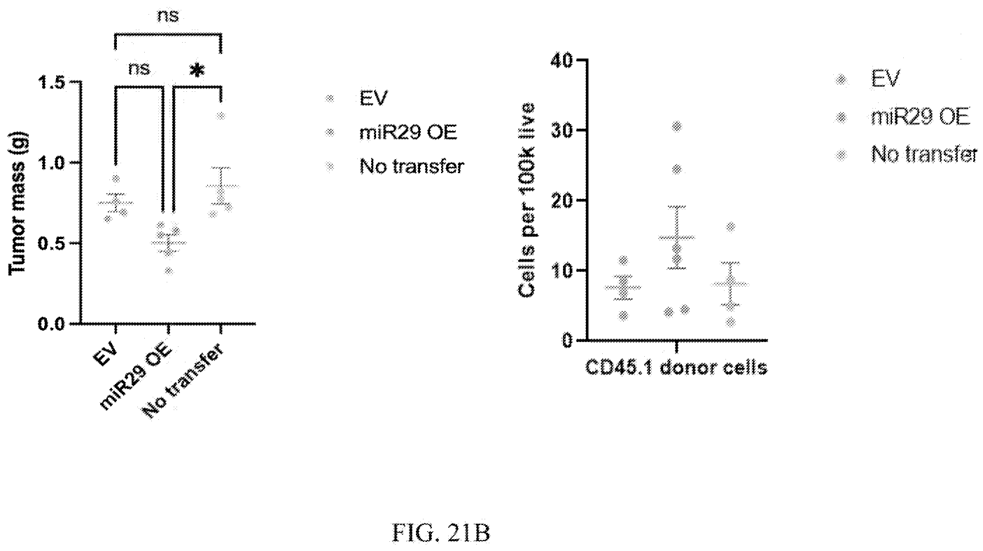

WT CD8 T cells (not antigen-specific) from CD45.1 donor mice were transduced with miR29-OE retrovirus or EV, flow sorted, and adoptively transferred to tumor-bearing KPC mice (FIG. 21A). Specifically, C57BL/6 mice were anesthetized with Ketamine: Xylazine (10:1) in sterile 0.9% NaCl saline. The abdomen was then wiped with iodine-based solution followed by alcohol wipes. The mice were placed on a clean sterile pad onto a warming pad. A paramedian incision was made in the abdomen using scissors and the pancreas and spleen were externalized with assistance of curved forceps. Then, 10 μL of cell suspension containing 25,000 LSL-KrasG12D/+; LSL-Trp53R172H/+; Pdx1Cre (KPC-6694c2) tumor cells in Matrigel R: were injected directly into the pancreas using a Hamilton™ microliter syringe. The pancreas and spleen were then returned to the intra-abdominal cavity in the anatomical position and the peritoneum sutured with Vicryl 5-0 suture and the skin closed with skin staples. Immediately after surgery, 300 μL of sterile saline was injected subcutaneous for fluid maintenance and buprenorphine 0.05-0.1 mg/kg was given subcutaneously for pain management. Mice were continuously monitored post-operatively and staples were removed two weeks post-surgery. Mice were sacrificed when the tumor volume for mice in each group (i.e., KPC, KC) achieved 1 cm3 (albeit at different time points), and tumor and spleen samples were collected for subsequent analysis. No animals were excluded from subsequent analysis. Results showed that CD8+ T cells that overexpressed miR29 limited tumor growth (FIG. 21B), demonstrating that miR-29a promotes anti-tumor responses in an orthotopic pancreatic tumor model.

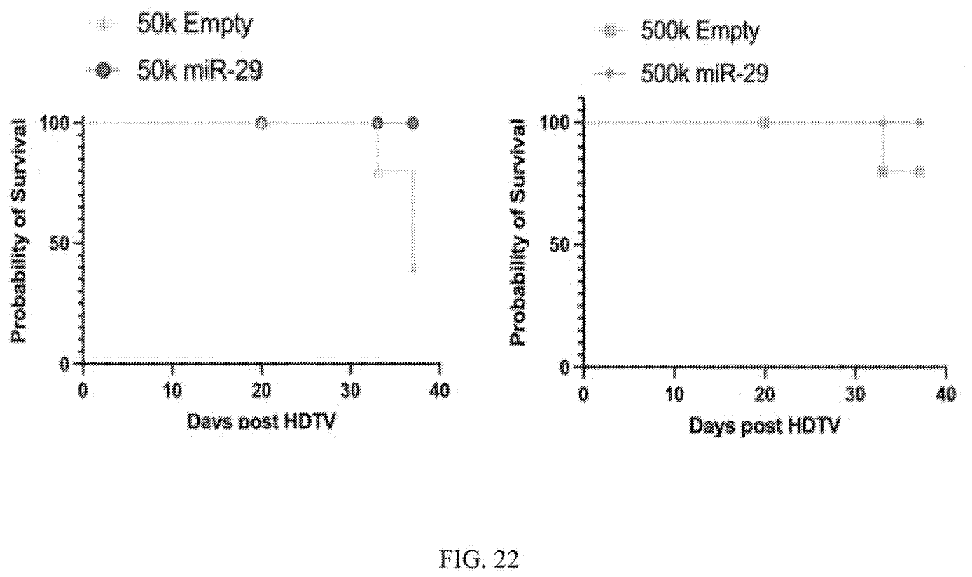

Hydrodynamic injections to model hepatocellular carcinoma were done in wild-type C57/BL6 female mice as previously described (Tschaharganeh D F et al., Cell. 2014: 158 (3): 579-592. Briefly, plasmid mix consisting of MYC transposon (10 mg), sleeping beauty transposase (2 mg), and sgRNA/Cas9 containing plasmid (10 mg) was injected in a single mouse in 2 mL saline. BSO treatments were done as described previously (Kramer R A et al., Int J Radiat Oncol Biol Phys. 1989: 16 (5): 1325-1329). Animals were randomized prior to treatments and tumor measurements were done in a blinded manner i.e., person injecting/measuring tumors did not know the experimental conditions. Hepatocellular carcinoma was induced on day 0 and miR-29-OE or empty control cells transferred on day 25 (50,000 or 500,000 RV-transduced (VEX+) sorted cells per mouse), as indicated (FIG. 22).

Results demonstrated that antigen-specific or non-antigen specific miR-29a overexpressing CD8 T cells provide a benefit for control of tumor growth in three separate preclinical tumor models: B16 melanoma, KPC orthopic pancreatic tumor model, and hepatocellular carcinoma tumor model. These results have therapeutic implications for CAR T cell development.

### ENUMERATED EMBODIMENTS

The following enumerated embodiments are provided, the numbering of which is not to be construed as designating levels of importance.

Embodiment 1 provides a modified immune cell or precursor cell thereof, comprising a nucleic acid encoding a chimeric antigen receptor (CAR) and a miR-29a microRNA, wherein the CAR comprises: an antigen binding domain domain that specifically binds a tumor associated antigen (TAA), a transmembrane domain, and an intracellular domain.

Embodiment 2 provides the modified immune cell of embodiment 1, wherein the transmembrane domain comprises a transmembrane region of a protein selected from the group consisting of a type I transmembrane protein, an alpha chain of a T cell receptor, a beta chain of a T cell receptor, a zeta chain of a T cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9.

Embodiment 3 provides the modified immune cell of any of the preceding embodiments, wherein the transmembrane domain comprises a CD8 transmembrane region.

Embodiment 4 provides the modified immune cell of any of the preceding embodiments, wherein the intracellular domain comprises a costimulatory domain of a protein selected from the group consisting of a TNFR superfamily member, CD27, CD28, 4-1BB (CD137), OX40 (CD134), CD30, CD40, PD-1, ICOS, lymphocyte function-associated antigen-1 (LFA-1), CD2, CD5, CD7, LIGHT, NKG2C, B7-H3, a ligand that specifically binds with CD83, DAP10, DAP12, Lck, Fas, and any derivative or variant thereof.

Embodiment 5 provides the modified immune cell of embodiment 4, the costimulatory domain is 4-1BB (CD137).

Embodiment 6 provides the modified immune cell of any of the preceding embodiments, wherein the intracellular domain comprises a signaling domain of a protein selected from the group consisting of CD3 zeta, FcyRIII, FcsRI, a cytoplasmic tail of an Fc receptor, an immunoreceptor tyrosine-based activation motif (ITAM) bearing cytoplasmic receptor, TCR zeta, FcR gamma, FcR beta, CD3 gamma, CD3 delta, CD3 epsilon, CD5, CD22, CD79a, CD79b, and CD66d.

Embodiment 7 provides the modified immune cell of any of the preceding embodiments, wherein the intracellular domain comprises a signaling domain of CD3 zeta.

Embodiment 8 provides the modified immune cell of any of the preceding embodiments, wherein the CAR further comprises a hinge domain.

Embodiment 9 provides the modified immune cell of embodiment 8, wherein the hinge domain is from a protein selected from the group consisting of an Fc fragment of an antibody, a hinge region of an antibody, a CH2 region of an antibody, a CH3 region of an antibody, an artificial spacer sequence, an amino acid hinge sequence of CD8, and any combination thereof.

Embodiment 10 provides the modified immune cell of embodiment 8 or 9, wherein the hinge domain is a CD8 hinge domain.

Embodiment 11 provides the modified immune cell of any of the preceding embodiments, wherein the modified cell is a modified T cell.

Embodiment 12 provides the modified immune cell of any of the preceding embodiments, wherein the modified immune cell is autologous.

Embodiment 13 provides the modified immune cell of any of the preceding embodiments, wherein the tumor-associated antigen is CD19.

Embodiment 14 provides the modified immune cell of any of the preceding embodiments, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Embodiment 15 provides a modified T cell comprising a nucleic acid encoding a chimeric antigen receptor (CAR) and a miR-29a microRNA, wherein the CAR comprises: an antigen binding domain domain that specifically binds a tumor associated antigen (TAA), a transmembrane domain, and an intracellular domain.

Embodiment 16 provides the modified T cell of claim 15, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Embodiment 17 provides an isolated nucleic acid sequence encoding a chimeric antigen receptor (CAR) and a miR-29a microRNA, wherein the CAR comprises: an antigen binding domain domain that specifically binds a tumor associated antigen (TAA), a transmembrane domain, and an intracellular domain.

Embodiment 18 provides the isolated nucleic acid of embodiment 17, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Embodiment 19 provides an expression construct comprising the isolated nucleic acid of embodiment 17 or 18.

Embodiment 20 provides the expression construct of embodiment 19, wherein the expression construct further comprises an EF-la promoter.

Embodiment 21 provides the expression construct of embodiment 20, wherein the expression construct further comprises a woodchuck hepatitis virus posttranscriptional regulatory element (WPRE).

Embodiment 22 provides the expression construct of embodiment 19, wherein the expression construct is a viral vector selected from the group consisting of a retroviral vector, a lentiviral vector, an adenoviral vector, and an adeno-associated viral vector.

Embodiment 23 provides the expression construct of embodiment 19, wherein the expression construct is a lentiviral vector.

Embodiment 24 provides the expression construct of embodiment 23, wherein the expression construct is a self-inactivating lentiviral vector.

Embodiment 25 provides a method of treating cancer in a subject in need thereof, comprising administering to the subject a therapeutically effective composition comprising the modified immune cell of any one of embodiments 1-14.

Embodiment 26 provides a method of treating a cancer in a subject in need thereof, the method comprising administering to the subject a therapeutically effective amount of a composition comprising a modified T cell comprising a nucleic acid encoding a chimeric antigen receptor (CAR) and a miR-29a microRNA, wherein the CAR comprises an antigen binding domain domain that specifically binds a tumor associated antigen (TAA), a transmembrane domain, and an intracellular domain.

Embodiment 27 provides the method of embodiment 26, wherein the modified T cell is autologous.

Embodiment 28 provides the method of embodiment 26, wherein the tumor associated antigen is CD19.

Embodiment 29 provides an isolated T cell compising a nucleic acid vector encoding a miR29-29a which is operably linked to a promoter.

Embodiment 30 provides the isolated T cell of embodiment 29, wherein the promoter is constitutive.

Embodiment 31 provides the isolated T cell of embodiment 29, wherein the promoter is inducible.

Embodiment 32 provides the isolated T cell of embodiment 29, wherein the promoter drives the expression of miR-29a such that the function of the isolated T cell is altered.

Embodiment 33 provides the isolated T cell of embodiment 29, wherein the T cell is a CD8 T cell.

Embodiment 34 provides the isolated T cell of embodiment 29 wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

Embodiment 35 provides a method of enhancing an immune response to an immunotherapy in a subject in need thereof, the method comprising contacting one or more immune effector cells of the subject with a nucleic acid vector encoding a miR-29a microRNA prior to treatment with the immunotherapy such that the immune effector cells express a high level of miR-29a microRNA as compared to uncontacted immune effector cells, thereby enhancing the effect of the immunotherapy.

Embodiment 36 provides the method of embodiment 35, wherein the immune effector cells are T cells.

Embodiment 37 provides the method of embodiment 36, wherein the T cells are CD8+ T cells.

Embodiment 38 provides the method of embodiment 35, wherein the high level of miR-29a renders the immune effectors cells resistant to immune exhaustion.

Embodiment 39 provides a method of treating a chronic infection in a subject in need thereof, the method comprising contacting one or more immune effector cells of the subject with a nucleic acid vector encoding a miR-29a microRNA such that the immune effector cells express a high level of miR-29a microRNA as compared to uncontacted immune effector cells, thereby treating the chronic infection.

Embodiment 40 provides the method of embodiment 39, wherein the immune effector cells are T cells.

Embodiment 41 provides the method of embodiment 40, wherein the T cells are CD8+ T cells.

Embodiment 42 provides the method of any of embodiments 35-41, wherein the nucleic acid vector comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A modified immune cell or precursor cell thereof, comprising a nucleic acid encoding a chimeric antigen receptor (CAR) and a miR-29a microRNA, wherein the CAR comprises:
an antigen binding domain domain that specifically binds a tumor associated antigen (TAA), a transmembrane domain, and an intracellular domain.

2. The modified immune cell of claim 1, wherein the transmembrane domain comprises a transmembrane region of a protein selected from the group consisting of a type I transmembrane protein, an alpha chain of a T cell receptor, a beta chain of a T cell receptor, a zeta chain of a T cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9.

3. The modified immune cell claim 1, wherein the transmembrane domain comprises a CD8 transmembrane region.

4. The modified immune cell of claim 1, wherein the intracellular domain comprises a costimulatory domain of a protein selected from the group consisting of a TNFR superfamily member, CD27, CD28, 4-1BB (CD137), OX40 (CD134), CD30, CD40, PD-1, ICOS, lymphocyte function-associated antigen-1 (LFA-1), CD2, CD5, CD7, LIGHT, NKG2C, B7-H3, a ligand that specifically binds with CD83, DAP10, DAP12, Lck, Fas, and any derivative or variant thereof.

5. The modified immune cell of claim 1, wherein the costimulatory domain is 4-1BB (CD137).

6. The modified immune cell of claim 1, wherein the intracellular domain comprises a signaling domain of a protein selected from the group consisting of CD3 zeta, FcyRIII, FcsRI, a cytoplasmic tail of an Fc receptor, an immunoreceptor tyrosine-based activation motif (ITAM) bearing cytoplasmic receptor, TCR zeta, FcR gamma, FcR beta, CD3 gamma, CD3 delta, CD3 epsilon, CD5, CD22, CD79a, CD79b, and CD66d.

7. The modified immune cell of claim 1, wherein the intracellular domain comprises a signaling domain of CD3 zeta.

8. The modified immune cell of claim 1, wherein the CAR further comprises a hinge domain.

9. The modified immune cell of claim 8, wherein the hinge domain is from a protein selected from the group consisting of an Fc fragment of an antibody, a hinge region of an antibody, a CH2 region of an antibody, a CH3 region of an antibody, an artificial spacer sequence, an amino acid hinge sequence of CD8, and any combination thereof.

10. The modified immune cell of claim 8, wherein the hinge domain is a CD8 hinge domain.

11. The modified immune cell of claim 1, wherein the modified cell is a modified T cell.

12. The modified immune cell of claim 1, wherein the modified immune cell is autologous.

13. The modified immune cell of claim 1, wherein the tumor-associated antigen is CD19.

14. The modified immune cell of claim 1, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

15. A modified T cell comprising a nucleic acid encoding a chimeric antigen receptor (CAR) and a miR-29a microRNA, wherein the CAR comprises:
an antigen binding domain domain that specifically binds a tumor associated antigen (TAA), a transmembrane domain, and an intracellular domain.

16. The modified T cell of claim 15, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

17. An isolated nucleic acid sequence encoding a chimeric antigen receptor (CAR) and a miR-29a microRNA, wherein the CAR comprises:
an antigen binding domain domain that specifically binds a tumor associated antigen (TAA), a transmembrane domain, and an intracellular domain.

18. The isolated nucleic acid of claim 17, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

19. An expression construct comprising the isolated nucleic acid of claim 17.

20. The expression construct of claim 19, wherein the expression construct further comprises an EF-1α promoter.

21. The expression construct of claim 20, wherein the expression construct further comprises a woodchuck hepatitis virus posttranscriptional regulatory element (WPRE).

22. The expression construct of claim 19, wherein the expression construct is a viral vector selected from the group consisting of a retroviral vector, a lentiviral vector, an adenoviral vector, and an adeno-associated viral vector.

23. The expression construct of claim 19, wherein the expression construct is a lentiviral vector.

24. The expression construct of claim 23, wherein the expression construct is a self-inactivating lentiviral vector.

25. A method of treating cancer in a subject in need thereof, comprising administering to the subject a therapeutically effective composition comprising the modified immune cell of claim 1.

26. A method of treating a cancer in a subject in need thereof, the method comprising
administering to the subject a therapeutically effective amount of a composition comprising a modified T cell comprising a nucleic acid encoding a chimeric antigen receptor (CAR) and a miR-29a microRNA,
wherein the CAR comprises an antigen binding domain domain that specifically binds a tumor associated antigen (TAA), a transmembrane domain, and an intracellular domain.

27. The method of claim 26, wherein the modified T cell is autologous.

28. The method of claim 26, wherein the tumor associated antigen is CD19.

29. An isolated T cell comprising a nucleic acid vector encoding a miR29-29a which is operably linked to a promoter.

30. The isolated T cell of claim 29, wherein the promoter is constitutive.

31. The isolated T cell of claim 29, wherein the promoter is inducible.

32. The isolated T cell of claim 29, wherein the promoter drives the expression of miR-29a such that the function of the isolated T cell is altered.

33. The isolated T cell of claim 29, wherein the T cell is a CD8 T cell.

34. The isolated T cell of claim 29, wherein the nucleic acid comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.

35. A method of enhancing an immune response to an immunotherapy in a subject in need thereof, the method comprising contacting one or more immune effector cells of the subject with a nucleic acid vector encoding a miR-29a microRNA prior to treatment with the immunotherapy such that the immune effector cells express a high level of miR-29a microRNA as compared to uncontacted immune effector cells, thereby enhancing the effect of the immunotherapy.

36. The method of claim 35, wherein the immune effector cells are T cells.

37. The method of claim 36, wherein the T cells are CD8+ T cells.

38. The method of claim 35, wherein the high level of miR-29a renders the immune effectors cells resistant to immune exhaustion.

39. A method of treating a chronic infection in a subject in need thereof, the method comprising contacting one or more immune effector cells of the subject with a nucleic acid vector encoding a miR-29a microRNA such that the immune effector cells express a high level of miR-29a microRNA as compared to uncontacted immune effector cells, thereby treating the chronic infection.

40. The method of claim 39, wherein the immune effector cells are T cells.

41. The method of claim 40, wherein the T cells are CD8+ T cells.

42. The method of claim 35, wherein the nucleic acid vector comprises a nucleotide sequence at least 80%, 85%, 90%, 95%, 96%, 97%, 98%, 99%, or 100% identical to any of SEQ ID NOs: 1, 27, 28, 29, 30, 31, 32, and 33.


SEQUENCE LISTING (39 sequences)

SEQ ID NO: 1 - DNA, 10321 bp, synthetic construct
  source 1..10321: mol_type = other DNA
gtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatggcgcgtctcgaggcatgctctcccatcaataacaaattcagtgacatcagtttatgaatatatgaaatttgccaaagctctgtttagaccactgagtaactcacagctaggtttcaacttttcctttctaggttgtcttgggtttattgtaagagagcattatgaagaaaaaaatagatcataaagcttcttcaggaagctggtttcatatggtggtttagatttaaatagtgattgtctagcaccatttgaaatcagtgttcttgggggagaccagctgcgctgcactaccaacagcaaaagaagtgaatgggacagctctgaagtatttgaaagcaacagcaggatggctgtgagaacctgcctcacatgtagctgaccccttcctcacccctgccaacagtggtggcatatatcacaaatggcagtcaggtctctgcactggcggatccaactgtgatcgaaagttttccaaaaataagttgtgtctgtattgaacatgaacagactttcttcttgtcattattctctaacaatactgcataacaattatttgcatacatttgcattgcattaagtattctaagtaatctagagacgatttaaagtatacgggaggatgtgtgtaggttgtatgcaaatactacaccattttctatcagagacttgagcatctgtggattttggtatccaaggggctttctggaaccaatccctcaaggataccaagggatgaatgtaattgtacaggatatcgcattgttggaattttatacttctttgtggaataaacctatagcacttaatagatagtacagactcattccattgtgcctgggttaaagagcccaatgtatgctggatttagtaagatttgggccctcccaaccctcacgaccttctgtgaccccttagaggatgactgatttcttttggtgttcagagtcaatataattttctagcaccatctgaaatcggttataatgattggggaagagcaccatgatgctgactgctgagaggaaatgtattggtgaccgttggggccatggacaagaactaagaaaacaaatgcaaagcaataatgcaaaggtgatttttcttcttccagtttctaagttgaatttcactgacctgaattgcatgtggtctcgagggcgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctctagagccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgg

SEQ ID NO: 2 - protein, 5 aa, synthetic construct
  REPEAT 1..5: note = repeat n times, where n is an integer of 1
GSGGS

SEQ ID NO: 3 - protein, 4 aa, synthetic construct
  source 1..4: note = repeat n times, where n is an integer of 1
  REPEAT 1..4
GGGS

SEQ ID NO: 4 - protein, 5 aa, synthetic construct
  REPEAT 1..5: note = repeat n times, where n is an integer of 1
GGGGS

SEQ ID NO: 5 - protein, 4 aa, synthetic construct
GGSG

SEQ ID NO: 6 - protein, 5 aa, synthetic construct
GGSGG

SEQ ID NO: 7 - protein, 5 aa, synthetic construct
GSGSG

SEQ ID NO: 8 - protein, 5 aa, synthetic construct
GSGGG

SEQ ID NO: 9 - protein, 5 aa, synthetic construct
GGGSG

SEQ ID NO: 10 - protein, 5 aa, synthetic construct
GSSSG

SEQ ID NO: 11 - protein, 5 aa, synthetic construct
GGGGS

SEQ ID NO: 12 - protein, 15 aa, synthetic construct
GGGGSGGGGSGGGGS

SEQ ID NO: 13 - protein, 15 aa, synthetic construct
GGGGSGGGGSGGGGS

SEQ ID NO: 14 - DNA, 45 bp, synthetic construct
  source 1..45: mol_type = other DNA
ggtggcggtggctcgggcggtggtgggtcgggtggcggcggatct

SEQ ID NO: 15 - protein, 5 aa, synthetic construct
DKTHT

SEQ ID NO: 16 - protein, 4 aa, synthetic construct
CPPC

SEQ ID NO: 17 - protein, 15 aa, synthetic construct
CPEPKSCDTPPPCPR

SEQ ID NO: 18 - protein, 12 aa, synthetic construct
ELKTPLGDTTHT

SEQ ID NO: 19 - protein, 10 aa, synthetic construct
KSCDKTHTCP

SEQ ID NO: 20 - protein, 7 aa, synthetic construct
KCCVDCP

SEQ ID NO: 21 - protein, 7 aa, synthetic construct
KYGPPCP

SEQ ID NO: 22 - protein, 15 aa, synthetic construct
EPKSCDKTHTCPPCP

SEQ ID NO: 23 - protein, 12 aa, synthetic construct
ERKCCVECPPCP

SEQ ID NO: 24 - protein, 17 aa, synthetic construct
ELKTPLGDTTHTCPRCP

SEQ ID NO: 25 - protein, 12 aa, synthetic construct
SPNMVPHAHHAQ

SEQ ID NO: 26 - protein, 15 aa, synthetic construct
EPKSCDKTYTCPPCP

SEQ ID NO: 27 - DNA, 1150 bp, synthetic construct
  source 1..1150: mol_type = other DNA
ctcgaggcatgctctcccatcaataacaaattcagtgacatcagtttatgaatatatgaaatttgccaaagctctgtttagaccactgagtaactcacagctaggtttcaacttttcctttctaggttgtcttgggtttattgtaagagagcattatgaagaaaaaaatagatcataaagcttcttcaggaagctggtttcatatggtggtttagatttaaatagtgattgtctagcaccatttgaaatcagtgttcttgggggagaccagctgcgctgcactaccaacagcaaaagaagtgaatgggacagctctgaagtatttgaaagcaacagcaggatggctgtgagaacctgcctcacatgtagctgaccccttcctcacccctgccaacagtggtggcatatatcacaaatggcagtcaggtctctgcactggcggatccaactgtgatcgaaagttttccaaaaataagttgtgtctgtattgaacatgaacagactttcttcttgtcattattctctaacaatactgcataacaattatttgcatacatttgcattgcattaagtattctaagtaatctagagacgatttaaagtatacgggaggatgtgtgtaggttgtatgcaaatactacaccattttctatcagagacttgagcatctgtggattttggtatccaaggggctttctggaaccaatccctcaaggataccaagggatgaatgtaattgtacaggatatcgcattgttggaattttatacttctttgtggaataaacctatagcacttaatagatagtacagactcattccattgtgcctgggttaaagagcccaatgtatgctggatttagtaagatttgggccctcccaaccctcacgaccttctgtgaccccttagaggatgactgatttcttttggtgttcagagtcaatataattttctagcaccatctgaaatcggttataatgattggggaagagcaccatgatgctgactgctgagaggaaatgtattggtgaccgttggggccatggacaagaactaagaaaacaaatgcaaagcaataatgcaaaggtgatttttcttcttccagtttctaagttgaatttcactgacctgaattgcatgtggtctcgag

SEQ ID NO: 28 - RNA, 64 nt, synthetic construct
  source 1..64: mol_type = other RNA
atgactgatttcttttggtgttcagagtcaatataattttctagcaccatctgaaatcggttat

SEQ ID NO: 29 - DNA, 64 bp, synthetic construct
  source 1..64: mol_type = other DNA
atgactgatttcttttggtgttcagagtcaatataattttctagcaccatctgaaatcggttat

SEQ ID NO: 30 - RNA, 22 nt, synthetic construct
  source 1..22: mol_type = other RNA
actgatttcttttggtgttcag

SEQ ID NO: 31 - DNA, 22 bp, synthetic construct
  source 1..22: mol_type = other DNA
actgatttcttttggtgttcag

SEQ ID NO: 32 - RNA, 22 nt, synthetic construct
  source 1..22: mol_type = other RNA
tagcaccatctgaaatcggtta

SEQ ID NO: 33 - DNA, 22 bp, synthetic construct
  source 1..22: mol_type = other DNA
tagcaccatctgaaatcggtta

SEQ ID NO: 34 - RNA, 81 nt, synthetic construct
  source 1..81: mol_type = other RNA
cttcaggaagctggtttcatatggtggtttagatttaaatagtgattgtctagcaccatttgaaatcagtgttcttggggg

SEQ ID NO: 35 - DNA, 81 bp, synthetic construct
  source 1..81: mol_type = other DNA
cttcaggaagctggtttcatatggtggtttagatttaaatagtgattgtctagcaccatttgaaatcagtgttcttggggg

SEQ ID NO: 36 - RNA, 24 nt, synthetic construct
  source 1..24: mol_type = other RNA
gctggtttcatatggtggtttaga

SEQ ID NO: 37 - DNA, 24 bp, synthetic construct
  source 1..24: mol_type = other DNA
gctggtttcatatggtggtttaga

SEQ ID NO: 38 - RNA, 23 nt, synthetic construct
  source 1..23: mol_type = other RNA
tagcaccatttgaaatcagtgtt

SEQ ID NO: 39 - DNA, 23 bp, synthetic construct
  source 1..23: mol_type = other DNA
tagcaccatttgaaatcagtgtt